SEQUENCE LISTING (40 sequences)

SEQ ID NO: 1 - DNA, 20 bp, artificial sequence
  PCR primer
gagcccctatcctaccttca

SEQ ID NO: 2 - DNA, 19 bp, artificial sequence
  PCR primer
ggttgcttcatccacacct

SEQ ID NO: 3 - DNA, 20 bp, artificial sequence
  PCR primer
acatcagattcggctcaagg

SEQ ID NO: 4 - DNA, 20 bp, artificial sequence
  PCR primer
ggactgcaaaccggaataag

SEQ ID NO: 5 - DNA, 18 bp, artificial sequence
  PCR primer
ttggagggcaaaggaagt

SEQ ID NO: 6 - DNA, 20 bp, artificial sequence
  PCR primer
gcagggaatgtggttcaagt

SEQ ID NO: 7 - DNA, 19 bp, artificial sequence
  PCR primer
cgcaagtcctcaaagcaca

SEQ ID NO: 8 - DNA, 20 bp, artificial sequence
  PCR primer
tcagtggtggcagtggtagt

SEQ ID NO: 9 - DNA, 20 bp, artificial sequence
  PCR primer
gatggggaagtagaacagca

SEQ ID NO: 10 - DNA, 21 bp, artificial sequence
  PCR primer
cggagaggtaagcaggtatca

SEQ ID NO: 11 - DNA, 18 bp, artificial sequence
  PCR primer
ttggacgtgcctttacga

SEQ ID NO: 12 - DNA, 20 bp, artificial sequence
  PCR primer
tgctgaacatgcacccatac

SEQ ID NO: 13 - DNA, 20 bp, artificial sequence
  PCR primer
ctgttccaactcttgctcca

SEQ ID NO: 14 - DNA, 20 bp, artificial sequence
  PCR primer
ggcatttcctctggttcact

SEQ ID NO: 15 - DNA, 20 bp, artificial sequence
  PCR primer
tgaggttagggcgaaatggt

SEQ ID NO: 16 - DNA, 20 bp, artificial sequence
  PCR primer
aatcgggtacaactggtgct

SEQ ID NO: 17 - DNA, 20 bp, artificial sequence
  PCR primer
cttatgggctgtgccatctt

SEQ ID NO: 18 - DNA, 20 bp, artificial sequence
  PCR primer
gtccgaactttctgctgctc

SEQ ID NO: 19 - DNA, 20 bp, artificial sequence
  PCR primer
ccttacttgcctctggaaca

SEQ ID NO: 20 - DNA, 20 bp, artificial sequence
  PCR primer
ctccctgaacctggacttga

SEQ ID NO: 21 - DNA, 20 bp, artificial sequence
  PCR primer
cgggagagtttgtgagttga

SEQ ID NO: 22 - DNA, 20 bp, artificial sequence
  PCR primer
agctagggaacaggaaagca

SEQ ID NO: 23 - DNA, 20 bp, artificial sequence
  PCR primer
gagagtgagggctttgagga

SEQ ID NO: 24 - DNA, 20 bp, artificial sequence
  PCR primer
ggagacctgggaaaagatgg

SEQ ID NO: 25 - DNA, 20 bp, artificial sequence
  PCR primer
tccctggagaagagctacga

SEQ ID NO: 26 - DNA, 20 bp, artificial sequence
  PCR primer
aggaaggaaggctggaagag

SEQ ID NO: 27 - DNA, 20 bp, artificial sequence
  PCR primer
aaactctggtggaggtccgt

SEQ ID NO: 28 - DNA, 21 bp, artificial sequence
  PCR primer
cttaccaaaagtggcccacta

SEQ ID NO: 29 - DNA, 1399 bp, homo sapiens
agtgtgaaatcttcagagaagaatttctctttagttctttgcaagaaggtagagataaagacactttttcaaaaatggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacacttaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagac

SEQ ID NO: 30 - DNA, 1925 bp, homo sapiens
cctggttccttattccggtttgcagtccccccagcacttgggagtaatttgaccgacaatgtcatgatcacagtagatattattccttcaggttggaattcagctgatggtaaaagtgataaaactaaaagtgcgccttcaagagatccagaaagattgcagaaaataaaagagagcctccttttagaggactcagaagaagaagaaggtgacttatgtagaatttgtcaaatggcagctgcatcatcatctaatttgctgatagagccatgcaagtgcacaggaagtttgcagtatgtccaccaagactgtatgaaaaagtggttacaggccaaaattaactctggttcttcattagaagctgtaaccacctgtgaactatgtaaagagaagttggagcttaacctggaggattttgatattcatgaactacatagagctcatgcaaatgaacaagctgagtatgagtttatcagctctggtctctacctagtggtgttattgcacttgtgcgaacaaagcttttctgatatgatgggaaatacaaatgaaccaagcacacgtgtccgatttattaaccttgcaagaactcttcaggcacatatggaagatctcgaaacttcagaggatgattccgaagaagacggagaccataacaggacatttgatattgcctaacttcatataagacagatggatgatctgtgaacataagtgtttattaaaaatggcaattaaatataaattacttttgtgggggaatgcctaataaatacattgactatatataaaatgaatatatacatacacatgtatgcctgtatatatatattcattctccagtgttgctgaattaaaattctgctggactttttaacatagcaaatccgatgtttataaactggtaatcaaaaaggttttttcttttaggtgagtgggaaagtattacccttgttttaaatatctaagcaatgcctatcaacccttttttgtgttatgattactgtagtcatatttatgaaaaaaggtttgtgttttactcttgctagtgagaaaagtgggacaaaatatacttttgaaataaaatgctatatggcacctaattattttttcttttaaaatgccttaagttgcagtctcattttgataatcatttgcttccagtgtttaaaaattaaaaaaagaatggggagaaggttatgagaagagcattattaagtttccaaatttaatttgaattccaaattcacctagcaataaaatctaatttttaaaaagtatataaatataaaatgtataaatgatggatagatttttgtattgatttgcaaaatgcagattatatttgataggctatagtatgtagatattccttttaggaatattacagctgtaaattatatgagacttgccagtcaaatgctatttggtttaaaaaaattattgcaatctcaagttaatggaatatttttaaatcccacattcagagtttaaaacactggttttcaatgtgttttttagtgttgtcacttgtttatagataaatatataaataacctgtttggatcctggtcctttttaactgttccttggtaattctgagcatttatttgatgacttaatatttttcactacctttggagaacagatgaacattattcaccatgaatggatctatactgtgtggtcatgagttgtgtatacttccataacactgtatttttcttctgtcagtacccttaggatacactttaaaacaccttaaggtctgatgttatggcaacaaactactttttcaaacctaaataggaaccatgtaatttctcaaaagtgattgaacagtttgcccacacttagtttgttggtcttatgtaaaacattggctcaaaataaagtacacactgatttaaaaaaaaaaaaaaaaa

SEQ ID NO: 31 - DNA, 2385 bp, homo sapiens
atggccgacttcgatgatcgtgtgtcggatgaggagaaggtacgcatagctgctaaattcatcactcatgcacccccaggggaatttaatgaagtattcaatgacgttcggctactacttaataatgacaatctcctcagggaaggggcagcacatgcatttgcccagtataacatggatcagttcacgcctgtgaagatagaaggatatgaagatcaggtcttaattacagagcacggtgacctgggtaatagcagatttttagatccaagaaacaaaatttcctttaaatttgaccacttacggaaagaagcaagtgacccccagccagaagaagcagatggaggtctgaagtcttggagagaatcctgtgacagtgctttaagagcctatgtgaaagaccattattccaacggcttctgtactgtttatgctaaaactatcgatgggcaacagactattattgcatgtattgaaagccaccagtttcagcctaaaaacttctggaatggtcgttggagatcagagtggaagttcaccatcacaccacctacagcccaggtggttggcgtgcttaagattcaggttcactattatgaagatggcaatgttcagttggttagtcataaagatgtacaggattcactaactgtttcgaatgaagcccaaactgccaaggagtttattaaaatcatagagaatgcagaaaatgagtatcagacagcaattagtgaaaactatcaaacaatgtcagataccacattcaaggccttgcgccgccagcttccagttacccgcaccaaaatcgactggaacaagatactcagctacaagattggcaaagaaatgcagaatgcttaaaggctgaatgtaggattcttcagtatgtggaaagacaaggattcaacgtgtggtcatatgataaataagtgatttataaacaagagtgatattttgctagggctttcaaagttaaccggttttctagcctcatggaatactgttgaacctatagcgttgtcttgattcttttgtgttctctgccttgtaattttctgttactgctatatctacgtgtaaatctttttttctttttttttttttttttttttcttttttggttaattctgccacatttaatgttggtgagagagtgatctatcctaatgacatttactgtttaaaaaagtttcctagccatgaagccctgctactgatttagacaaggtattatggtcattactttgtacccctatccttccaagcacttctggtacttcagtcgtttttactgatccaccaacacctaaagaggctatgctacagtctctagctaaatggaagacacattcatccttctccctctgactgctttgatcatcatttattgcatcgtcatatcatatttatcgcatctcataactaactttctaaagtttggattgggacttttcaggtcctttttggagggcaaaggaagttccagcttctctggggaacttgtttttaaatccaaagacttgaaccacattccctgcacatgaacatgtttgcttttatcccttctctcattggctccttcccatcttagtaccattgtagttatacatctgcattttttagaagcattttacccatttatttttttaaacattcaagaactgctgacgtactgtggatgtagagtataaaacttgaaaaatgcagatgttgaaggaataataggtatcttgtgctttaatactttatggcaggattgtactataagcaaatgaattaaacagctatgtaaatcataaagaaaaactaaaaatgaaccaaagtgaaaggataacttccaggcagtatctttctattgtaacctgttatttaaggaaatactagtgatttcttctaaataggatgtaaacttctttcaaattactcttcctcagtctgcctgccaagaactcaagtgtaactgtgataaaataacctttcccaggtatattcggcaggtatgtgtgtaatctcagaatacacaggtgacatagatatgatatgacaactggtaatggtggattcatttacattgtttacacttctatgaccaggccttaagggaaggtcagttttttaaaaaaccaagtagtgtcttcctacctatctccagatacatgtcaaaaagaaaaggtgtttgtgctccgttttgtttctgctcagtaatatagtcaagcaagtttgttccaggtgacccattgagctgtgtatgcatttttgtttatttcaataaaatatatttgtattatttgtccttcatactatccatccataccacactatcttctgtatcaggtagtctaatagaaatatacctgttttgttctaaaaaaaaaaaaaaaaaaa

SEQ ID NO: 32 - DNA, 3958 bp, homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 33 - DNA, 856 bp, homo sapiens
tccaccaccatcagatgcagttccctatttgtttaatgaagggatatataagctttctaatggtgtcttcagaaatttataaaatgtaaatactgatttgactggtctttaagatgtgtttaactgtgaggctatttaacgaatagtgtggatgtgattgtcatccagtattaagttcttagtcattgattttgtgtttaaaaaaaaataggaaagagggaaactgcagctttcattacagattccttgattggtaagctctccaaatgatgagttctagtaaactctgattttttgcctctggatagtagatctcgagcgtttatctcgggctttaattttgctaaagctgtgcacataatgtacaaacaaaaacacaacaaaagcattatttcaaaggggaaaattctacgggtgtcacacattatgggctatagtgtccatttctttaaagacagttatgccctttaaaggctttaaaaaagacggcctacctttgtttgcacaacaaaagttggcgtgattccccaacccccaaaaatatggtaattaacactttacctttcctgtgtggagttacacctcttttatgtgttcatttcgtaaaacggcggaaaaaaatcccctttggggtctcctaattttatacaacaacagggctttctaatttgtggacccctgtgtataacaagattttcggacgacactccggtgtagactgctacaggacttggcaatggcacacgtcaatgtcaacaaaatccggtttacaagaccacctgcgcttccctaaaaggtatttgaaccacaggtttggtcataagtcctaatttaaaacaacaatacagacaacata

SEQ ID NO: 34 - DNA, 2097 bp, homo sapiens
tccctctgctatggctcttcctcagtagaaacaactggcaacaaaattcaagtttatgattcattcatcagcaaacatgtgagaatcatctacaaagaaccaagaattgtgagagaaagcgacctcaagatacaactggcaactgaggaaaaggcctcaattcaacaagagctaacaagcttgggagtttatttcggaatctttaaaagactcttctgcttacccacaatctgggatccactgcaggaaaacaaaaaaggaaaacttcatttaaaagaagcaagaagtaaaatgggacaaattgggaatgtttaagtctctgaaactctgcactgaaaagaaaataagattgataacttaagcttaacattctgaggcataaagaaacattaactttggagtattcatcttgactactgaaatacaagtttagaagacaagtggtttcattctggtcacagatcacagcttttctttaaatttataatcctatgggttggactcgttgactgtattttttaaaggttgctcgtcagttaactgagccttggaattcatggattttctaaagactaacaaatgaaaatattttcctgttgaagaacccagcggaaattttacagcaacaaatttcatgtttcttttgggtatttctgagaaaaaggaaatatttataaaaccatccaaagatccagataatttgcaaataaattggaggttatagaggttataatctgaatcccaaaggagactgcagctgatgaaagtgcttccaaactgaaaattggacgtgcctttacgatggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctgaaataaaaccattaggactcactgggacagaactttcaagttccttcaactgtgaaaagtgtctttttggacaaactatttttccacctccaaaagaaattaacacatggacattttaaagtctttagtataaagaaaatttgtattcaatgtgttaagcattaacatgtattttatttgtgtatccactccatctgatttttctgagccattttgatttgttccttcattaaaaaaaatctcttaaagttaaaaaaaaaaa

SEQ ID NO: 35 - DNA, 2250 bp, homo sapiens
gaggccgcggcggggaaaatggcggacgggaaggcgggagacgagaagcctgaaaagtcgcagcgagctggagccgccggaggacctgaagaagaagcagaaaaacctgtgaaaactaagactgtttcttccagtaatggaggggaaagttccagtcgcagcgctgagaagcgatcagctgaagaagaagctgccgacctcccaacaaagcctacaaagatctccaagtttggatttgccataggtagtcagacgacaaagaaagcatcagccatatccatcaaacttggatcaagtaagcctaaagaaactgttccaactcttgctccaaaaactctttcagtagcagcagcttttaatgaagatgaagatagtgaaccagaggaaatgcctccagaagcaaagatgaggatgaagaatattggaagggatacaccaacatcagctggaccaaactccttcaataaaggaaagcatgggttttctgataaccagaagctgtgggagcgaaatataaaatctcatcttggaaatgtccatgaccaagacaattaaatgatgttttgaaattggggtgtggggtgggtgtaaagttaaaaggaacagtttccttttttaaagaatggtataagactatctttggagccgctttttttttctttttcatttttttaaaagattgagtggtacactaataaatgagagtttgaaattagaggtaatttatgttttatatacagatttcaagacatttgctaattttgtagtttcatgtgattagtttccaaaggttacagataataaagaaatcagaaatggtacctttttaagaattgcatatttttttagacacaactattagcacattaagagggaagcaaagttactgtctatttaaaactgcaagcagttaactctcttaactcccttattacctaaacttgtctggctcccaggaacagccttatagagagagggagtattgtattgggaagaaaatgttactgaactattgactgaaagtaaatttagataaaatacagcttttttccttatgggcatttgttttgtttcaagtcatcataaactaggtattgcattgctatccgtggatacagacgcttagctcttaaaagattttttttttatgtaaactgttgaatatttgaaatagtccacttcaccttaatgggtcttgtctatcttcattagtcttcaaagaaaaaccatttgctaccaaagtaaatcagtattttgaatgtgcttctcttgttttttgtttattagctagttcctgtaagcatttccaccagaacttgaggcaaatcgtaaggaagctgtttcttttaaaacacaaaccaccaccaaaaatttaaatgtacatattgcttaagtatttggctgtttttattttttaaaaggtataaacaccaaaaaaaaaattaacattgtatgaagatggaaaataagaagatgcactttctgtaactttgtctaaggatttaaattactaacttatgaactccaatttgaattgaacttaactatcggctttcttactggtaaaattatatggtttattttaaatgcgtacatattgaccaatggcctctgaaaaagcacattttagatactgaaattgaaggaaagaaaatgcatcttcaaacattttttggaatctcaccacatatactttgttagatttgtgtattgtagggtgtttgttttgtatttttgtattgtatatgaactttttttaaatgtgacagttaaacacatctttaaaagcatagtcacagacaaaagcatacagtataaaaatttccttgaaaactcctacaatattatatttggaggcagcttcagactgttttattggtggtagctgcttgctgaggtcttttagttggtaataactccagagaagcagcctgtgtatattcctaacactttgttcactagcatttaagtttagaataagcccaagtaagacaatggaaatgtatatagaactcttagttcttacatgatttaattatatcgatacatgaatttaacttactttaatgtaggcaaactatcaattttttgtccattttcctgtttgttaaaataacatacctctcctacgtattattttcttgacccaaatgaaatattaacctaaggtcaagctgggagagagaaatgactgagatgaatgtctttactaaagtaccaataaatttgtcaaactcaaaaaaaaaaaaa

SEQ ID NO: 36 - DNA, 502 bp, homo sapiens
  misc_feature (177)..(177): n is a, c, g, or t
  misc_feature (420)..(420): n is a, c, g, or t
  misc_feature (482)..(482): n is a, c, g, or t
tttatattaataacagatatgtttattattacatatccatcagtgcggctttcaataccatttgaaacatgcatatcatcctagagacgatcggttttccagtgcttcttatctgatacacattactgcaaagccattataaattactgaggaggtatttggttaaaaaaataaacnataaatcatactgtccatatgaatcaactcttgttactggacaagagtttaacagtatttatctggtaattcctatgttaactggaaaacatcatggatgtattgccatatttcctttttatgttggtgcaaccttctacattttttgcagttttataaagtcattattgtagcaggtagtttgatgcaaagattgtactcaatttatgaaagtccaacaaacctctaaatattttaaattanaccaattatcaatgaaaaactcactgacacacataattttttgaaatacattttttgaattncatacttactcatggcagta

SEQ ID NO: 37 - DNA, 4314 bp, homo sapiens
ggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaaaaaaaaaaaaaaaaaaaaaaaa

SEQ ID NO: 38 - DNA, 588 bp, homo sapiens
  misc_feature (526)..(526): n is a, c, g, or t
  misc_feature (534)..(534): n is a, c, g, or t
  misc_feature (541)..(541): n is a, c, g, or t
  misc_feature (584)..(584): n is a, c, g, or t
gaaaaaaaaatagtgttttattaactaccacactgttataatacactttaaacgtacaataaggtagcctttaaatttgaggtggtcttaagaataacaaatgaacagaattccaaatttttgaaataggtgaactgctgcagttacaggtatacatttaggaaaactgtatagctcttacaagaccagcaatgtaactttattttgtacatttttgaattgaaaatataaacaataattaaaaaataaaaagaaaatacagcataataaaaaacatacgcttctcaattaaatgtactggatacatataaattttaagggaagaagcaaaaaaggaaaatgattgatatttaagtgcagactgactacctagacaaaaaaaaaaaaagacttaaaaaaatatcataaaacctctagttcttctatgactaatatccatatggttggagtatcgtcactatggaagtgatttggtatgtttgcatatgtacactttactggtaatttacatgatggcttttaaggncctggganacatggnttttggaaccagaatgggttaaaatcactggtaaacccattcncctt

SEQ ID NO: 39 - DNA, 2310 bp, homo sapiens
acttccgtttttgctgggtaggcgacccggacggagggccggcgaggtgcggggtctggtgatgcgagctgcgcctctcggcaagatttcgcgctgcccatcccgggccctttcatcagtaatcggtagtggatcactctgccaagcggcaggaagaattaaggaaacgacaaggagacgctcggctctctcccgcttggctccttgcggcctcctcttcccttcgctccggcccggtgaaactgaacttataatcgtcactggattgtaagtacccgaggcgaagagagctcgctgagccctgattttttgagtgtctttgttccgggagagtttgtgagttgaaagtatctctgctgggctttctgggccgaaaaccgttccgggggagccgccatttgctttcctgttccctagctagctagctagctctctccgcgttgtccggcagcggcacctagaggttgggacttggcattgcatctgatttaatgaacttaagtctgtgaataagcctttgtgttaacgactggtattcggtcacagcatatttagagaaaagacttggagcttaaataaaaactaaggcaaaatagacgcttagctgctgatctacagagaacttcttgtaattaaaagatttcaattcatagcaaactggtgttttaaactattgcagtagctggaactttttagtgtaaccagcatttattggagaagtgaatcacaaggaaataaagatgagtaaaagcaaagatgatgctcctcacgaactggagagccagtttatcttacgtctgcctccagaatatgcctctactgtgagaagggcagtacagtctggtcatgtcaacctcaaggacagactgacaattgagttacatcctgatgggcgtcatggaatcgtcagagtggaccgtgttccattggcctcaaaattagtagacctgccctgtgttatggaaagcttgaaaaccattgataaaaaaactttttacaagacagctgatatctgtcagatgcttgtatccacagttgatggtgatctctatcctcctgtggaggagccagttgctagcactgatcctaaagcaagcaagaaaaaggataaggacaaagagaaaaagtttatctggaaccacggaattactctgcctctaaagaatgtcaggaagagaaggttccggaagacagcaaagaagaaatatattgaatctccagatgttgaaaaagaagtgaaacgattgctgagtacagatgctgaagctgttagtactcggtgggaaataattgccgaagatgaaacaaaggaggcagaaaatcaaggcctggatatctcttctccaggaatgtctggtcacaggcagggccatgactcattagaacatgatgagcttcgggagatattcaatgacctcagcagcagcagtgaggatgaagatgagacccagcatcaagatgaagaagatataaacatcattgacacggaggaagatctggagagacagctacaggacaagctaaatgaatcagatgaacagcaccaggaaaatgaaggaaccaatcagctggttatgggaattcagaagcagattgacaacatgaaaggcaagctccaagagacccaggacagggcaaaacgacaagaggatctcatcatgaaagtggaaaatctggctctcaagaacagatttcaggctgtactggatgagctcaaacaaaaggaagaccgagaaaaggagcaactcagctctttgcaagaggagctagaatcactcctagagaagtaaaaagaactgatatttaatttcagtcttcagactggtcagcattagaaaattcttggctttattgtactgggtattaagaccttgctcttcctagtccttttaatgctgtgtgttctgttaagttctttcatttgtttgtaattttgtttttcagcaaatttatattgttttgctaggtgttcatcctataagaagcaggattgtataggcagaaaaatgattgtaggaaagttgcaggattagcggaatgtatggttcaaccttaattatagcttcattgcaggactttactgtttctccattttctagaagctgctgttgctgctttgtgatgacgtgagatcaataagaagaacctagtctagagacaatgatgctagtttgcatatgttttcctatgcaataattgttttcccagttattcaaagcagctttctatatgtagagatgcaaattattaagttgtttccaatacaataaataaaagcatctgtttttcactttaaaaaaaaaaaaaaaa

SEQ ID NO: 40 - DNA, 2183 bp, homo sapiens
ccagggcctgactaaacctggagactcgggtggccgaggggcttcataccagctgaagagcgacaagccgctggcagccgcggatctcaccgccgctcagggtttttagaacttcagccataaaaatgggcagaattttccttgatcatatcggtggtacccgtctgttttcttgtgcaaactgtgatacgatcctgaccaaccgctcagaactcatctccactcgtttcacaggcgccactggcagagcatttctttttaacaaggtagttaacctgcagtacagcgaagttcaagatcgggtcatgctcactggccgccacatggttcgagatgtgagctgcaaaaactgcaatagcaaactgggatggatctatgagtttgccactgaagacagccagcgatataaggaaggccgcgtgatcctggaacgtgctctagttcgagagagtgagggctttgaggagcatgtaccatctgataactcttgaagatacagagagaaatccatcttttcccaggtctccttcactgaaaacaaaaatctacttacatacactgtcaccttagcatcagagtcggattaatgaactgcggaacaagaggttgtgagaatctaagatggaacctttctttctttctttctttttttttaaattttgtattttccatccaacagcagtgtgtagagagaatattatgcagatgccgttaattttttaccctatgtttacatcttgaggcagcagagtctgtctgcagctatgtggtgagctatgtaaggaaaaaaatctgggctgttagagtgaaaaagtgtgttttatgtcaattgtgaaaggaaaatgttaggagtatggtttttaaacttgggcttcattttaaacttttttttttaaacccagttatttcacttgatttgctagcttcagagaagagatccgaatctgtgcccagcgctaaaggctcagtgttagcatggcttgtgctggccggtgtgccatattcttgttggagatgaaccgtagcaccagagcccattcttccttgtcagtcttggcccaaagatgtcaccattcctagttatttgtcaccacataattggtgttgattggaaactttttctgagatgggacagaactgctgggttgtctttttccatgtaacttaagcatagtaatataaataaagtaatagttggatgcttttggtcctgtgttgcttttaaaaacaccttataaaagaggagagtatttgataagcaattttcatagtagtaaagttttttttcatctcttaaactaaattgaccatgcatataatattctttgtttaaatgaaagcatactgttgaaacccgcagtgttgcatttagaaaacagttgaacagaatgtcaatgtgcattcatgcaaaaaaacatttaatctgcatctgttttagaaaagggggaaatgaagcaacttgtctaaaaatactgctttacaaagcatttcagcctttccccctcagttttgcattgattttttgacaagtctgtagagcctaatagtttccatcaaaggcctagatctcttatttagcatttttttcagctcttctctcagaagttcagctgttgaaacgaaaactgtactttgtaccctcacatacaaagggatcaaatttgacctggtgttattttagccccaaatttatgacattacacaatattaaaatgtaaatgtttctttacccaaactacttctagatattctagtatttgcttctggtggaattaaatgacggtaaaattggctaattatttgaatgaatgaatggatggatgttttgcatgctcaatttctaggtcctttgtctagaaaggaaatttgcctcagttgaattagtgaaatatttctgtcgttgatattaaaagtgacttctgagtacagttaagttcctcctatttgccactgggctgttggttagaagcataggtaactgattaagtaggtatgatactgcatttgaaataagtggacacaaactatcctttctccaccatggactcaatctgagaacaacagcattcatttccattcatttccatactggcttttgattatatgcagattcctagtagcatgccttacctacagcactatgtgcatttgctgtcacaataaagtatattttgtcttgcaaa

(19) United States
(12) Patent Application Publication
(10) Pub. No.: US 2007/0015183 A1
(43) Pub. Date: Jan. 18, 2007

(54) Biomarkers for huntington's disease

(75) Inventor: Dimitri Krainc, Boston, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: The General Hospital Corporation, Boston, MA (US)

(21) Appl. No.: 11/440,574

(22) Filed: May 25, 2006

Related U.S. Application Data
(60) Provisional application No. 60/687,134, filed on Jun. 3, 2005.

(51) Int. Cl. C12Q 1/68; A61K 38/12; A61K 31/19; A61K 31/165

(52) U.S. Cl. 435/6; 514/9; 514/575; 514/616

(57) ABSTRACT

The invention relates, in part, to specific genes and set of genes that are selectively expressed in Huntington's disease and their use for the diagnosis and staging of HD. Additionally, the selectively expressed genes are useful in methods to assess HD pathogenesis in cells, tissues, and subjects, and in the assessment of the efficacy of HD therapeutics.

### RELATED APPLICATIONS

This application claims priority under 35 U.S.C. §119(e) from U.S. provisional application Ser. No. 60/687,134, filed Jun. 3, 2005, the entire content of which is incorporated by reference herein.

### GOVERNMENT SUPPORT

This invention was made in part with government support under grant numbers NS002174 and NS045242 from the National Institutes of Health (NIH). The government may have certain rights in this invention.

### FIELD OF THE INVENTION

The invention relates, in part, to specific genes and set of genes that are selectively expressed in Huntington's disease (HD) and their use for the diagnosis and staging of HD. Additionally, the selectively expressed genes are useful in methods to assess HD pathogenesis in cells, tissues, and patients, and in the assessment of the efficacy of HD therapeutics.

### BACKGROUND OF THE INVENTION

A number of naturally occurring proteins have uninterrupted tracts of glutamine residues encoded by CAG triplet repeats. It now known that the expansion of the length of these uninterrupted tracts or regions of polyglutamine repeats in proteins is associated with specific neurodegenerative diseases. The expansion of polyglutamine tracts in proteins may become pathogenic if the polyglutamine tracts expand beyond a threshold length, which for most of the polyglutamine expansion-associated disorders is a length of approximately 35-40 residues. When the threshold is reached, the presence of the abnormal protein is associated with neurodegenerative diseases such as: Huntington's disease (HD).

It is known that Huntington's disease is characterized by mutant huntingtin protein with abnormal expansions of polyglutamine tracts in the coding region of huntingtin protein. In HD, abnormal expanded regions of CAG repeats have been identified in the coding region the huntingtin protein. HD is an autosomal dominant neurodegenerative disorder caused by the expanded CAG tract in the huntingtin gene. HD is characterized clinically by progressive motor impairment, cognitive decline, and various psychiatric symptoms with the typical age of onset in the third to fifth decades. Postmortem changes in HD brains include neuronal loss and gliosis, particularly in the cortex and the striatum. (Vonsattel JP et al. J Neuropathol Exp Neurol. 57: 369-384, 1998.

The onset of Huntington's disease is characterized by choreic movements that result from the selective involvement of medium spiny neurons of the striatum. As HD progresses, more regions of the brain and spinal cord of the patient become involved. The severity of the symptoms and progression of HD varies from patient to patient, in part due to fact that the length of the expanded polyglutamine region correlates with the severity of the symptomatic presentation. The presence of a longer expanded polyglutamine repeat may indicate a more severe type of HD than the presence of a less long expanded polyglutamine repeat. Thus, patients with longer expanded polyglutamine regions may have more severe clinical effects from the disease and may show an earlier age of onset than would patients with shorter expanded polyglutamine regions.

A decade of basic research since the genetic mutation was discovered has demonstrated that the pathogenesis of HD seems to involve the recruitment of multiple biochemical pathways. (Harjes, P. et al., Trends Biochem. Sci. 28: 425-433, 2003) More recently, transcriptional deregulation and loss of function of transcriptional co-activator proteins have been implicated in HD pathogenesis. (Sugars, K. L. et al., Trends in Genetics 19: 233-238, 2003; Dunah, A. W. et al., Science 296: 2238-2243, 2002). Numerous microarray studies showed alterations in mRNA levels of a large number of genes in the brain of HD mice, suggesting that huntingtin may interfere with transcriptional mechanisms common to many genes (Luthi-Carter, R. et al., Hum. Mol. Genet. 9: 1259-1271, 2000; Luthi-Carter, R., et al., Hum. Mol. Genet. 11: 1911-1926, 2002). Mutant huntingtin has been shown to specifically disrupt activator-dependent transcription in early stages of HD pathogenesis. (Dunah, A. W. et al., Science 296: 2238-2243, 2002) In addition, recent studies in cell culture, yeast, and Drosophila models of polyglutamine disease have indicated that HDAC inhibitors might provide a useful class of agents to ameliorate the transcriptional changes in HD. (Steffan, J. S. et al., Nature 413: 739-743, 2001; McCampbell, A. et al., Proc. Natl. Acad. Sci. USA 98: 15179-15184, 2001; Hughes, R. E. et al., Proc. Natl. Acad. Sci. USA 98: 13201-13206, 2001; Hockly, E. et al., Proc. Natl. Acad. Sci. USA 100: 2041-2046, 2003; and Ferrante, R. J. et al., J. Neurosci. 23: 9418-9427, 2003.

Although it is possible to diagnose HD, there are very limited treatment options available for patients diagnosed with HD. Additionally, it is not currently possible to adequately stage HD, to closely follow its onset, progression, or to monitor the effect of candidate therapeutic agents on HD. The lack of effective treatments for HD means that even with a definitive initial diagnosis, the therapeutic options are quite limited.

### SUMMARY OF THE INVENTION

The invention described herein relates to the identification of genes that are specifically expressed in Huntington's disease (HD). The invention described herein relates to the identification of gene sequences that have altered expression in subjects having Huntington's disease. These gene sequences are useful as biomarkers of Huntington's disease (HD) and their expression can be assessed in blood. The newly identified Huntington's disease biomarker gene sequences (see Table 1) may be used singly, in pairs, in larger groups, and combined with other gene sequences having differential expression in HD as markers for HD, and most preferably are used in the diagnosis, treatment assessment, and for staging the progression, regression, or onset of HD in humans. A listing of 322 genes whose expression has been examined with respect to HD disease are provided in Tables 1a and 1b, with their Genbank Accession numbers and their Affymetrix and Amersham probe names and descriptions.

The newly identified HD biomarkers can be used in reproducible and minimally invasive assays with which to assess the cellular changes that are associated with HD. These newly identified biomarkers for HD may be used in methods to monitor the effects of therapeutic compounds on the status and/or progression and of HD in cells, tissues, and subjects. The newly identified HD biomarker genes can be used in pre-clinical screening of compounds before a full clinical trial is initiated. This may be useful for developing treatments for asymptomatic subjects who carry an HD mutation because there is no clinical phenotype to be monitored in these subjects. In some embodiments of the invention, the subject is asymptomatic for HD. In certain embodiments of the invention, the subject is symptomatic for HD.

According to one aspect of the invention, methods for diagnosing Huntington's Disease (HD) are provided. The methods include detecting in a biological sample that includes blood from a subject a level of expression of one or more HD biomarker genes, comparing the level of expression of the one or more HD biomarker genes with a control level of expression of the one or more HD biomarker genes, wherein a difference between the level of expression of the one or more HD biomarker genes in the biological sample and the control level of expression of the one or more HD biomarker genes is diagnostic for HD in the subject. In some embodiments, the level of expression is detected for 2, 3, 4, 5, 6, 7, 8, 9, 10, 11, 12, 13, 14, 15, 16, 17, 18, 19, 20 or more biomarker genes. In some embodiments, the level of expression is detected for 2 or more biomarker genes, including any number of biomarker genes up to and including 322 biomarker genes. In some embodiments, the subject is asymptomatic for HD. In certain embodiments, the subject is symptomatic for HD. In certain embodiments, the biological sample is a peripheral blood sample. In certain embodiments, the level of expression is detected by determining the amount of mRNA transcribed from the one or more HD biomarker genes. In some embodiments, the method of determining the amount of mRNA includes reverse transcription polymerase chain reaction (RT-PCR) amplification. In some embodiments, the level of expression is detected by determining the amount of a polypeptide encoded by the one or more HD biomarker genes. In certain embodiments, the method of determining the amount of the polypeptide includes contacting the biological sample with an antibody that specifically binds to a polypeptide encoded by one of the one ore more HD biomarker genes. In some embodiments, the HD biomarker genes include ANXA1, AXOT, CAPZA1, HIF1A, JJAZ1, P2Y5, PCNP, ROCK1 (p160ROCK), SF3B1, SP3, TAF7 and YIPPEE. In some embodiments, the HD biomarker genes consist of ANXA1, AXOT, CAPZA1, HIF1A, JJAZ1, P2Y5, PCNP, ROCK1 (p160ROCK), SF3B1, SP3, TAF7 and YIPPEE. In certain embodiments, a higher level of expression of one or more HD biomarker genes in the biological sample compared with the control level of expression of the one or more HD biomarker genes is diagnostic for HD. In some embodiments, a lower level of expression of one or more HD biomarker genes in the biological sample compared with the control level of expression of the one or more HD biomarker genes is diagnostic for HD.

According to another aspect of the invention, methods for determining onset, progression, or regression, of Huntington's disease (HD) in a subject are provided. The methods include detecting in a first biological sample including blood from a subject a level of expression of one or more HD biomarker genes, detecting in a second biological sample including blood and obtained from the subject at a time later than the first biological sample, the level of expression of the one or more HD biomarker genes, comparing the level of expression of the one or more HD biomarker genes in the first biological sample with the level of expression of the one or more HD biomarker genes in the second biological sample, wherein a change in the level between the first biological sample and the second biological sample is an indication of onset, progression, or regression of HD. In some embodiments, the level of expression is detected for 2, 3, 4, 5, 6, 7, 8, 9, 10, 11, 12, 13, 14, 15, 16, 17, 18, 19, 20 or more biomarker genes. In some embodiments, the level of expression is detected for 2 or more biomarker genes, including any number of biomarker genes up to and including 322 biomarker genes. In some embodiments, the subject is asymptomatic for HD. In certain embodiments, the subject is symptomatic for HD. In some embodiments, the biological sample is a peripheral blood sample. In some embodiments, the level of expression is detected by determining the amount of mRNA transcribed from the one or more HD biomarker genes. In certain embodiments, the method of determining the amount of mRNA includes reverse transcription polymerase chain reaction (RT-PCR) amplification. In some embodiments, the level of expression is detected by determining the amount of a polypeptide encoded by the one or more HD biomarker genes. In certain embodiments, the method of determining the amount of the polypeptide includes contacting the biological sample with an antibody that specifically binds to a polypeptide encoded by one of the one or more HD biomarker genes. In some embodiments, the HD biomarker genes include ANXA1, AXOT, CAPAZ1, HIF1A, JJAZ1, P2Y5, PCNP, ROCK1 (p160ROCK), SF3B1, SP3, TAF7 and YIPPEE. In some embodiments, the HD biomarker genes consist of ANXA1, AXOT, CAPAZ1, HIF1A, JJAZ1, P2Y5, PCNP, ROCK1 (p160ROCK), SF3B1, SP3, TAF7 and YIPPEE. In some embodiments, a lower level of expression of one or more HD biomarker genes in the first biological sample compared with the level of expression of the one or more of the HD biomarker genes in the second biological sample indicates onset or progression of HD in the subject. In certain embodiments, a higher level of expression of one or more HD biomarker genes in the first biological sample compared with the level of expression of the one or more HD biomarker genes in the second biological sample indicates regression of HD in the subject. In some embodiments, a lower level of expression of one or more HD biomarker genes in the first biological sample compared with the level of expression of the one or more of the RD biomarker genes in the second biological sample indicates regression of RD in the subject. In certain embodiments, a higher level of expression of one or more RD biomarker genes in the first biological sample compared with the level of expression of the one or more RD biomarker genes in the second biological sample indicates onset or progression of RD in the subject.

According to yet another aspect of the invention, methods for selecting a course of treatment of a subject having or suspected of having Huntington's disease are provided. The methods include detecting in a biological sample including blood from a subject a level of expression of one or more RD biomarker genes, comparing the level of expression of the one or more RD biomarker genes to a control level of expression of the one or more RD biomarker genes, determining the stage and/or type of RD of the subject based on the difference in the level of expression of one or more RD biomarker genes in the sample compared to the level of expression of the one or more RD biomarker genes to the control level of expression, and selecting a course of treatment for the subject appropriate to the stage and/or type of RD of the subject. In certain embodiments, the level of expression is detected for 2, 3,4, 5, 6, 7, 8, 9, 10, 11, 12, 13, 14, 15, 16, 17, 18, 19, 20 or more biomarker genes. In some embodiments, the level of expression is detected for 2 or more biomarker genes, including any number of biomarker genes up to and including 322 biomarker genes. In some embodiments, the subject is asymptomatic for RD. In certain embodiments, the subject is symptomatic for HD. In some embodiments, the biological sample is a peripheral blood sample. In certain embodiments, the level of expression is detected by determining the amount of mRNA transcribed from the one or more RD biomarker genes. In some embodiments, the method of determining the amount of mRNA includes reverse transcription polymerase chain reaction (RT-PCR) amplification. In some embodiments, the level of expression is detected by determining the amount of a polypeptide encoded by the one or more RD biomarker genes. In some embodiments, the method of determining the amount of the polypeptide includes contacting the biological sample with an antibody that specifically binds to a polypeptide encoded by one of the one or more RD biomarker genes. In certain embodiments, the RD biomarker genes include ANXA1, AXOT, CAPAZ1, HIF1A, JJAZ1, P2Y5, PCNP, ROCK1 (p160ROCK), SF3B1, SP3, TAF7 and YIPPEE. In some embodiments, the RD biomarker genes consist of ANXA 1, AXOT, CAPZA 1, HIF1A, JJAZ1, P2Y5, PCNP, ROCK1 (p160ROCK), SF3B1, SP3, TAF7 and YIPPEE. In some embodiments, a higher level of expression of one or more HD biomarker genes in the biological sample compared with the control level of expression of the one or more HD biomarker genes indicates a more advanced stage and/or more severe type of HD in the biological sample compared with the control. In some embodiments, a lower level of expression of one or more HD biomarker genes in the biological sample compared with the control level of expression of the one or more HD biomarker genes indicates a more advanced stage and/or more severe type of HD in the biological sample compared with the control. In certain embodiments, the treatment selected is a histone deacetylase (HDAC) inhibitor.

According to another aspect of the invention, methods for monitoring responses to treatment in a subject with Huntington's disease (HD) are provided. The methods include detecting in a biological sample including blood from a subject that has received treatment for HD, a level of expression of one or more HD biomarker genes, comparing the level of expression of the one or more HD biomarker genes with a control level of expression of the one or more HD biomarker genes, wherein a change in the level of expression of one or more of the HD biomarker genes in the biological sample relative to the control level of expression of the HD biomarker genes indicates that the subject is responding to the treatment for HD. In some embodiments, the level of expression is detected for 2, 3, 4, 5, 6, 7, 8, 9, 10, 11, 12, 13, 14, 15, 16, 17, 18, 19, 20 or more biomarker genes. In some embodiments, the level of expression is detected for 2 or more biomarker genes, including any number of biomarker genes up to and including 322 biomarker genes. In some embodiments, the subject is asymptomatic for HD. In certain embodiments, the subject is symptomatic for HD. In certain embodiments, the biological sample is a peripheral blood sample. In some embodiments, the level of expression is detected by determining the amount of mRNA transcribed from the one or more HD biomarker genes. In some embodiments, the method of determining the amount of mRNA includes reverse transcription polymerase chain reaction (RT-PCR) amplification. In some embodiments, the level of expression is detected by determining the amount of a polypeptide encoded by the one or more HD biomarker genes. In certain embodiments, the method of determining the amount of the polypeptide includes contacting the sample with an antibody that specifically binds to a polypeptide encoded by one of the one or more HD biomarker genes. In some embodiments, the HD biomarker genes include ANXA1, AXOT, CAPZA1, HIF1A, JJAZ1, P2Y5, PCNP, ROCK1 (p160ROCK), SF3B1, SP3, TAF7 and YIPPEE. In some embodiments, the HD biomarker genes consist of ANXA1, AXOT, CAPZA1, HIF1A, JJAZ1, P2Y5, PCNP, ROCK1 (p160ROCK), SF3B1, SP3, TAF7 and YIPPEE. In some embodiments, a lower level of expression of one or more HD biomarker genes in the biological sample compared with the control level of expression of the one or more HD biomarker genes indicates that the subject is responding to the treatment for HD. In certain embodiments, a higher level of expression of one or more HD biomarker genes in the biological sample compared with the control level of expression of the one or more HD biomarker genes indicates that the subject is responding to the treatment for HD. In certain embodiments, the treatment is a histone deacetylase (HDAC) inhibitor. In some embodiments, the HDAC inhibitor is a short-chain fatty acid, an hydroxamic acid, an epoxyketone-containing cyclic tetrapeptide, a non-epoxyketone-containing cyclic tetrapeptide, a benzamide, a depudecin ((2R,3S,4S,5E,7S,8S,9R)-2,9-Dihydroxy-3,4,7,8-diepoxyundeca-5, 10-diene), or an organosulfur compound. In some embodiments, the HDAC inhibitor is a short-chain fatty acid. In certain embodiments, the short-chain fatty acid is butyrate, phenylbutyrate, or valproate. In some embodiments, the short-chain fatty acid is phenylbutyrate. In certain embodiments, the HDAC inhibitor is a hydroxamic acid. In some embodiments, the hydroxamic acid is a trichostatin, trichostatin A (TSA), suberoylanilide hydroxamic acid (SAHA) or its derivatives, oxamflatin, azelaic bishydroxamic acid (ABHA), Scriptaid, pyroxamide, or a propenamide. In some embodiments, the HDAC inhibitor is an epoxyketone-containing cyclic tetrapeptide. In certain embodiments, the epoxyketone-containing cyclic tetrapeptide is a trapoxin, HC-toxin, Chlamydocin, Diheteropeptin, WF-3161 (cyclo(L-Leu-L-Pip-L-Aoe-D-Phe) where Pip=pipecolic acid and Aoe=2-amino-8-oxo-9,10-epoxydecanoic acid), Cyl- 1, or Cyl-2. In some embodiments, the HDAC inhibitor is a non-epoxyketone-containing cyclic tetrapeptide. In some embodiments, the non-epoxyketone-containing cyclic tetrapeptide is FR901228, apicidin, or a cyclic-hydroxamic-acid-containing peptide (CHAP). In some embodiments, the HDAC inhibitor is a benzamide. In certain embodiments, the benzamide MS-275 (MS-27-275), CI-994 (N-acetyldinaline), M344 (4-Dimethylamino-N-(6-hydroxycarbamoylhexyl)-benzamide), or a benzamide analog.

According to yet another aspect of the invention, methods for diagnosing Huntington's Disease (HD) are provided. The methods include obtaining from a subject a biological sample, detecting in the biological sample a level of expression of two or more HD biomarker genes that include: ANXA1, AXOT, CAPZA1, HIF1A, JJAZ1, P2Y5, PCNP, ROCK1 (p160ROCK), SF3B1, SP3, TAF7, and YIPPEE, and wherein if two HD biomarker genes are selected, both of the HD biomarker genes are not ROCK1 (p160ROCK) and SP3, comparing the level of expression of the two or more HD biomarker genes with a control level of expression of the two or more HD biomarker genes, wherein a difference between the level of expression of the two or more HD biomarker genes in the biological sample and the control level of expression of the two or more HD biomarker genes is diagnostic for HD in the subject. In some embodiments, the level of expression is detected for 3, 4, 5, 6, 7, 8, 9, 10, 11, 12, 13, 14, 15, 16, 17, 18, 19, 20 or more biomarker genes. In some embodiments, the level of expression is detected for 3 or more biomarker genes, including any number of biomarker genes up to and including 322 biomarker genes. In some embodiments, the subject is asymptomatic for HD. In certain embodiments, the subject is symptomatic for HD. In some embodiments, the biological sample is a blood sample. In certain embodiments, the biological sample is a tissue sample. In some embodiments, the tissue sample is a neuronal tissue sample. In certain embodiments, the level of expression is detected by determining the amount of mRNA transcribed from the two or more HD biomarker genes. In some embodiments, the method of determining the amount of mRNA includes reverse transcription polymerase chain reaction (RT-PCR) amplification. In some embodiments, the level of expression is detected by determining the amount of a polypeptide encoded by a HD biomarker gene. In some embodiments, the method of determining the amount of the polypeptide includes contacting the sample with an antibody that specifically binds to a polypeptide encoded by one of the two or more HD biomarker genes. In certain embodiments, the HD biomarker genes consist of: ANXA1, AXOT, CAPZA1, HIF1A, JJAZ1, P2Y5, PCNP, ROCK1 (p160ROCK), SF3B1, SP3, TAF7, and YIPPEE. In some embodiments, a higher level of expression of two or more HD biomarker genes the biological sample compared with the control level of expression of the two or more HD biomarker genes is diagnostic for HD. In some embodiments, a lower level of expression of two or more HD biomarker genes in the tissue sample compared with the control level of expression is diagnostic for HD. In some embodiments, a lower level of expression of two or more HD biomarker genes the biological sample compared with the control level of expression of the two or more HD biomarker genes is diagnostic for HD.

According to another aspect of the invention, methods determining onset, progression, or regression, of Huntington's disease (HD) in a subject are provided. The methods include detecting in a first biological sample from a subject a level of expression of two or more HD biomarker genes that include: ANXA1, AXOT, CAPZA1, HIF1A, JJAZ1, P2Y5, PCNP, ROCK1 (p160ROCK), SF3B1, SP3, TAF7, and YIPPEE, and wherein if two HD biomarker genes are selected, both of the HD biomarker genes are not ROCK1 (p160ROCK) and SP3, detecting in a second biological sample obtained from the subject at a time later than the first biological sample, the level of expression of the two or more HD biomarker genes, comparing the level of expression of the two or more HD biomarker genes in the first biological sample with the level of expression of the two or more HD biomarker genes in the second biological sample, wherein a change in the level between the first biological sample and the second biological sample is an indication of onset, progression, or regression of HD. In certain embodiments, the level of expression is detected for 3, 4, 5, 6, 7, 8, 9, 10, 11, 12, 13, 14, 15, 16, 17, 18, 19, 20 or more biomarker genes. In some embodiments, the level of expression is detected for 3 or more biomarker genes, including any number of biomarker genes up to and including 322 biomarker genes. In some embodiments, the subject is asymptomatic for HD. In certain embodiments, the subject is symptomatic for HD. In some embodiments, wherein the first and second biological sample are blood samples. In some embodiments, the first and second biological sample are tissue samples. In certain embodiments, the tissue samples are neuronal tissue samples. In some embodiments, the level of expression is detected by determining the amount of mRNA transcribed from the two or more HD biomarker genes. In certain embodiments, the method of determining the amount of mRNA includes reverse transcription polymerase chain reaction (RT-PCR) amplification. In some embodiments, the level of expression is detected by determining the amount of a polypeptide encoded by the two or more HD biomarker genes. In some embodiments, the method of determining the amount of the polypeptide includes contacting the sample with an antibody that specifically binds to a polypeptide encoded one of the two or more HD biomarker genes. In some embodiments, the HD biomarker genes consist of: ANXA1, AXOT, CAPZA1, HIF1A, JJAZ1, P2Y5, PCNP, ROCK1 (p160ROCK), SF3B1, SP3, TAF7, and YIPPEE. In certain embodiments, a lower level of expression of two or more HD biomarker genes in the first biological sample compared with the level of expression of the two or more HD biomarker genes in the second biological sample indicates onset or progression of HD in the subject. In some embodiments, a higher level of expression of two or more HD biomarker genes in the first biological sample compared with the level of expression of the two or more HD biomarker genes in the second biological sample indicates regression of HD in the subject. In some embodiments, a higher level of expression of two or more HD biomarker genes in the first biological sample compared with the level of expression of the two or more HD biomarker genes in the second biological sample indicates onset or progression of HD in the subject. In certain embodiments, a lower level of expression of two or more HD biomarker genes in the first sample compared with the level of expression of the two or more HD biomarker genes in the second sample indicates regression of HD in the subject.

According to yet another aspect of the invention, methods for selecting a course of treatment of a subject having or suspected of having Huntington's disease are provided. The methods include obtaining from the subject a biological sample, detecting in the biological sample a level of expression of two or more HD biomarker genes that include: ANXA1, AXOT, CAPZA1, HIF1A, JJAZ1, P2Y5, PCNP, ROCK1 (p160ROCK), SF3B1, SP3, TAF7, and YIPPEE, and wherein if two HD biomarker genes are selected, both of the HD biomarker genes are not ROCK1 (p160ROCK) and SP3, comparing the level of expression of the two or more HD biomarker genes to a control level of expression of the two or more HD biomarker genes determining the stage and/or type of HD of the subject based on the difference in the level of expression of two or more HD biomarker genes in the sample compared to the level of expression of the two or more HD biomarker genes to the control level of expression, and selecting a course of treatment appropriate to the stage and/or type of HD of the subject. In some embodiments, the level of expression is detected for 3, 4, 5, 6, 7, 8, 9, 10, 11, 12, 13, 14, 15, 16, 17, 18, 19, 20 or more biomarker genes. In some embodiments, the level of expression is detected for 3 or more biomarker genes, including any number of biomarker genes up to and including 322 biomarker genes. In some embodiments, the subject is asymptomatic for HD. In certain embodiments, the subject is symptomatic for HD. In certain embodiments, the biological sample is a blood sample. In some embodiments, the biological sample is a tissue sample. In some embodiments, the tissue sample is a neuronal tissue sample. In certain embodiments, the level of expression is detected by determining the amount of mRNA transcribed from the two or more HD biomarker genes. In some embodiments, the method of determining the amount of mRNA includes reverse transcription polymerase chain reaction (RT-PCR) amplification. In some embodiments, the level of expression is detected by determining the amount of a polypeptide encoded by the two or more HD biomarker genes. In some embodiments, the method of determining the amount of the polypeptide includes contacting the sample with an antibody that specifically binds to a polypeptide encoded by one of the two or more HD biomarker genes. In certain embodiments, the HD biomarker genes consist of: ANXA1, AXOT, CAPZA1, HIF1A, JJAZ1, P2Y5, PCNP, ROCK1 (p160ROCK), SF3 B1, SP3, TAF7, and YIPPEE. In some embodiments, a higher level of expression of two or more HD biomarker genes in the biological sample compared with the control level of expression of the two or more HD biomarker genes indicates a more advanced stage and/or more severe type of HD in the biological sample compared with the control. In certain embodiments, a higher level of expression of the two or more HD biomarker genes in the blood sample compared with control level of expression of the two or more HD biomarker genes indicates a more advanced stage and/or more severe type of HD in the biological sample compared with the control. In some embodiments, a lower level of expression of two or more HD biomarker genes in the biological sample compared with the control level of expression of the two or more HD biomarker genes indicates a more advanced stage and/or more severe type of HD in the biological sample compared with the control.

According to another aspect of the invention, methods for monitoring responses to treatment in a subject with Huntington's disease (HD) are provided. The methods include detecting in a biological sample from a subject that has received treatment for HD, a level of expression of two or more HD biomarker genes including: ANXA1, AXOT, CAPAZ1, HIF1A, JJAZ1, P2Y5, PCNP, ROCK1 (p160ROCK), SF3B1, SP3, TAF7, and YIPPEE, and wherein if two HD biomarker genes are selected, both of the HD biomarker genes are not ROCK1 (p160ROCK) and SP3, comparing the level of expression of the two or more HD biomarker genes with a control level of expression of the two or more HD biomarker genes, wherein a change in the level of expression of the two of more HD biomarker genes in the biological sample relative to the control level of expression of the two or more HD biomarker genes indicates that the subject is responding to the treatment for HD. In some embodiments, the level of expression is detected for 3, 4, 5, 6, 7, 8, 9, 10, 11, 12, 13, 14, 15, 16, 17, 18, 19, 20 or more biomarker genes. In some embodiments, the level of expression is detected for 3 or more biomarker genes, including any number of biomarker genes up to and including 322 biomarker genes. In some embodiments, the subject is asymptomatic for HD. In certain embodiments, the subject is symptomatic for HD. In some embodiments, the biological sample is a blood sample. In some embodiments, the biological sample is a tissue sample. In certain embodiments, the tissue sample is a neuronal tissue sample. In some embodiments, the level of expression is detected by determining the amount of mRNA transcribed from the two or more HD biomarker genes. In some embodiments, the method of determining the amount of mRNA includes reverse transcription polymerase chain reaction (RT-PCR) amplification. In some embodiments, the level of expression is detected by determining the amount of a polypeptide encoded by the two or more HD biomarker genes. In certain embodiments, the method of determining the amount of the polypeptide includes contacting the sample with an antibody that specifically binds to a polypeptide encoded by one of the two or more HD biomarker genes. In some embodiments, the HD biomarker genes consist of: ANXA1, AXOT, CAPZA1, HIF1A, JJAZ1, P2Y5, PCNP, ROCK1 (p160ROCK), SF3B1, SP3, TAF7, and YIPPEE. In some embodiments, a lower level of expression of two or more HD biomarker genes in the biological sample compared with the control level of expression of the two or more HD biomarker genes indicates that the subject is responding to the treatment for HD. In some embodiments, a higher level of expression of two or more HD biomarker genes in the biological sample compared with the control level of expression of the two or more HD biomarker genes indicates that the subject is responding to the treatment for HD. In certain embodiments, the treatment is a histone deacetylase (HDAC) inhibitor. In some embodiments, the HDAC inhibitor is a short-chain fatty acid, an hydroxamic acid, an epoxyketone-containing cyclic tetrapeptide, a non-epoxyketone-containing cyclic tetrapeptides, benzamide, depudecin ((2R,3S,4S,5E,7S,8S,9R)-2,9-Dihydroxy-3,4,7,8-diepoxyundeca-5,10-diene) or a organosulfur compound. In some embodiments, the HDAC inhibitor is a short-chain fatty acid. In some embodiments, the short-chain fatty acid is butyrate, phenylbutyrate, or valproate. In certain embodiments, the short-chain fatty acid is phenylbutyrate. In some embodiments, the HDAC inhibitor is a hydroxamic acid. In some embodiments, the hydroxamic acid is a trichostatin, trichostatin A (TSA), suberoylanilide hydroxamic acid (SAHA) or its derivatives, oxamflatin, azelaic bishydroxamic acid (ABHA), Scriptaid, pyroxamide, or propenamide. In some embodiments, the HDAC inhibitor is an epoxyketone-containing cyclic tetrapeptide. In certain embodiments, the epoxyketone-containing cyclic tetrapeptide is a trapoxin, HC-toxin, Chlamydocin, Diheteropeptin, WF-3161 (cyclo(L-Leu-L-Pip-L-Aoe-D-Phe) where Pip=pipecolic acid and Aoe=2-amino-8-oxo-9,10-epoxydecanoic acid), Cyl-l or Cyl-2. In some embodiments, the HDAC inhibitor is a non-epoxyketone-containing cyclic tetrapeptide. In some embodiments, the non-epoxyketone-containing cyclic tetrapeptide is FR901228, apicidin or a cyclic-hydroxamic-acid-containing peptide (CHAP). In certain embodiments, the HDAC inhibitor is a benzamide. In some embodiments, the benzamide is MS-275 (MS-27-275), CI-994 (N-acetyldinaline), M344 (4-Dimethylamino-N-(6-hydroxycarbamoylhexyl)-benzamide), or a benzamide analog.

These and other aspects of the invention will be described in further detail in connection with the detailed description of the invention.

### BRIEF DESCRIPTION OF THE DRAWINGS

FIG. 1 shows expression maps from cluster analysis of the 322 most differentially expressed genes on Affymetrix (FIG. 1A) and Amersham (FIG. 1B) microarrays. The genes were selected from 17 HD affected subjects and 14 healthy control subjects according to P value (P<0.0005), fold change (>1.8 or <0.6) and expression maximum greater than 100 (Affymetrix) or 1 (Amersham). Each column represents a sample and each row a gene. Colorgram depicts high (red) and low (green) relative levels of gene expression. The samples were normalized (median-polished) for each platform. Hierarchical clustering using cosine correlation with complete linkage was performed on the pool of all samples from both platforms to determine sample and gene clustering. The two groups were then separated in the display to compare the gene profiles between the two platforms (healthy control subjects C1-14, late presymptomatic carriers of the HD mutation P1-5, symptomatic HD patients S1-12).

FIG. 2 shows a graph representing the differential expression of a subset of genes and a table with a description of the 12 genes with representative gene symbols. FIG. 2A shows a graph of the differential expression of a subset of genes confirmed with quantitative real-time polymerase chair reaction (QRT-PCR). The upregulation of expression of the 12 selected genes in blood of HD patients was confirmed on the training set of 11 HD patients and 5 controls and a test set of 30 new HD patients and 25 controls using QRT-PCR. Values represent average fold change=2^{-(average ΔΔCt)} in mRNAs in HD patients relative to healthy control subjects (in every case P<0.05). Error bars are presented as (average fold change)×(2^{SE M}−1). Panel B shows gene descriptions of the 12 genes with representative gene symbols, GenBank accession numbers, LocusLink ID numbers, probe IDs for Affymetrix and Amersham microarrays. (ANXA1 is SEQ ID NO:29, AXOT is SEQ ID NO:30, CAPZA1 is SEQ ID NO:31, HIF1A is SEQ ID NO:32, JJAX1 is SEQ ID NO:33, P2Y5 is SEQ ID NO:34, PCNP is SEQ ID NO: 35, ROCK1 is SEQ ID NO:36, SF3B1 is SEQ ID NO:37, SP3 is SEQ ID NO:38, TAF7 is SEQ ID NO:39, and YIPPEE is SEQ ID NO:40).

FIG. 3 shows graphs representing results of principle component analysis (PCA) sets measuring differential gene expression in presymptomatic HD patients, symptomatic HD patients, and normal controls. FIG. 3A shows results of principal component analysis (PCA) in the training set confirmed the separation of the presymptomatic carriers of the HD mutation and HD patients from healthy control subjects. FIG. 3B shows results obtained from using the first two principal components from the QRT-PCR training set, PCA for the 12 genes in the test set confirmed the separation of symptomatic HD patients from healthy controls. FIG. 3C shows results indicating that early presymptomatic carriers of the HD mutation (average age 22.5±2.6) cluster predominantly with control samples whereas FIG. 3D shows results indicating that late presymptomatics (average age 39±6.1) mostly group with symptomatic HD patients. PCA was performed using the ΔCt values. For visualization in a two-dimensional plot the first two principal components for the training set data were chosen representing 81% of the variance in the training set. The subgroups are indicated according to their clinical classification (healthy control subjects ♦, early presymptomatics ▪, late presymptomatics ▴, symptomatic HD patients ●). FIG. 3E shows the graphic analysis of the individual genes revealed upregulation of expression with progression from early presymptomatic to symptomatic stage of HD. All of the 12 genes were significantly upregulated in symptomatic HD patients (▪) and late presymptomatic carriers of HD mutation (▪) compared to controls (p<0.05). In the early presymptomatic group (□) only annexin A1 (ANXA1), TAF7 and purinergic receptor P2Y (P2Y5) were significantly upregulated (p<0.05). Gene expression of the 12 selected genes in FIG. 3A-E was analyzed by QRT-PCR.

FIG. 4 shows graphs indicating that expression of the marker gene set was decreased in HD patients treated with phenylbutyrate. RNA was isolated from blood samples taken from 12 HD patients (HD 1 -HD 12) before treatment and after four weeks of treatment with phenylbutyrate. QRT-PCR analysis of gene expression of the 12 selected genes (FIG. 4A) showed significant decrease in expression after treatment in 10 out of 12 patients (P<0.05). In patients HD2 and HD 12 no changes were observed. The values are presented as average fold change=2^{-(average ΔΔCt)} for gene set in treated relative to untreated samples. Error bars represent (average fold change)×(2^{SEM}−1). In FIG. 4B, QRT-PCR analysis of each individual gene from the marker set in patients treated with phenylbutyrate showed statistically significant decrease in expression of 8 out of 12 genes (P<0.05) after treatment. The values represent average fold change=2^{-(average ΔΔCt)} for the samples after treatment compared to untreated samples. Error bars are presented as (average fold change)×(2^{SE M}−1)

FIG. 5 shows a graph indicating that genes with altered expression in HD blood are differentially expressed in HD brain. Expression of the 12 marker genes was analyzed in 5 postmortem HD caudate samples (Vonsatel grades 0-2) and 4 control samples using QRT-PCR. 7 genes were significantly upregulated in brain tissue samples from HD patients compared to controls (P<0.05). Values are presented as average fold change=2^{-(average ΔΔCt)} for HD brain samples compared to controls. Error bars are presented as (average fold change)×(2^{SE M}−1).

### DETAILED DESCRIPTION OF THE INVENTION

The invention described herein relates to the identification of gene sequences that have altered expression in subjects having Huntington's disease. These gene sequences are newly identified as useful biomarkers for Huntington's disease (HD). The HD biomarker gene sequences of the invention may be used singly, in pairs, in larger groups, and combined with other gene sequences having differential expression in HD as markers for HD, and most preferably can be used for the diagnosis, treatment assessment, and for staging the progression, regression, or onset of HD in humans. Surprisingly, the HD disease biomarkers of the invention can be assessed in peripheral blood samples thereby enabling non-invasive methods for diagnosis, staging, and therapeutic assessment in HD.

The invention relates to the identification of genes that are specifically expressed in Huntington's disease (HD). As used herein the term “biomarkers”, “HD disease biomarkers”, “biomarkers for HD”, and the like mean an HD-associated gene or an expression product (nucleic acid or polypeptide) of an HD-associated gene that can be used as an indicator or marker for HD in a cell, tissue, or subject. As used herein, “HD-associated genes” means genes the expression of which is associated with HD. Thus, the expression of an HD-associated gene is such that it reflects the presence, status, and/or stage of HD in the cell, tissue, or subject. As will be understood by those of skill in the art, the distribution and/or level of expression of some genes associated with HD may increase with the onset or progression of HD, and the expression of other HD-associated genes may decrease with the onset or progression of HD. In each case, the change in the distribution and/or level of expression of the gene is characteristic of HD.

The HD-associated genes of the invention are genes that are expressed in HD with a distribution or level over the course of the disease that differs from the expression of the HD-associated genes in cells, tissues, and/or subjects that are HD-free cells, tissues, and/or subjects. As used herein the term “level” means amount. As used herein, the term “distribution” means the cell and tissue location of expression of an HD-associated gene in a subject.

In all embodiments, human HD-associated genes and nucleic acid and polypeptide molecules expressed by the HD-associated genes, are preferred. It will be understood that the expression of some HD-associated genes of the invention during the course of HD may at times be the same as the expression of healthy controls, but the gene will still be an HD-associated gene if its expression differs from control expression at different stages of HD, for example, as the disease progresses. Thus, at the onset of HD, some HD-associated genes may be expressed at levels similar to that of HD-free controls, but the same HD-associated genes will have different expression in HD cells, tissues, and/or subjects than HD-free controls when the HD cells, tissues, and subjects are at other stages of HD. Thus, determining the level of expression of a group of HD-associated genes permits staging and status assessment of HD in cells, tissues, and subjects through the assessment of the differences and similarities between expression of members of a group of HD-associated genes.

HD-associated genes have been identified through screening of patients with HD. Gene expression was determined for a large number of genes through the measurement of mRNA levels in subjects known to have HD and control subjects (see Examples). The newly identified HD-associated genes and expressed nucleic acid molecules and encoded proteins thereof may be used as markers for HD in the diagnosis and treatment assessment of HD. In addition, sets of at least 2, 3, 4, 5, 6, 7, 8, 9, 10, 11, 12, 13, 14, 15, 16, 17, 18, 19, 20 or more (up to about 325) HD-associated genes and the expressed nucleic acid and polypeptides molecules thereof, may be used as markers in the diagnosis and treatment assessment of HD in humans. In some embodiments, the level of expression is detected for 2 or 3 or more biomarker genes, including any number of biomarker genes up to and including 322 biomarker genes.

The invention permits assessment of expression of an HD-associated gene at various stages of HD. For example, assessment of HD-associated gene expression can be done in asymptomatic cells, tissues, or subjects as well as in cells, tissues, and subjects that have symptoms or pathologic signs of HD. The identification of the HD-associated genes, nucleic acids and polypeptides of the invention allow their use in HD diagnostic assays and kits. In addition, sets of at least two, or at least three of the HD-associated genes of the invention or the expressed nucleic acid and/or polypeptide molecules thereof, may be used in HD diagnostic assays and kits. Such assays and kits are useful to detect HD in human subjects, and for staging the onset, progression, and/or regression of HD in subjects. The methods and kits described herein may also be used to evaluate treatments for HD.

As used herein, a subject is a mammal, preferably a human, non-human primate, cow, horse, pig, sheep, goat, dog, cat, or rodent. In all embodiments, human subjects are particularly preferred. In some embodiments, the subject is suspected of having HD. In other embodiments the subject has been diagnosed with HD. In some embodiments of the invention, the subject is asymptomatic for HD. In certain embodiments of the invention, the subject is symptomatic for HD. The symptoms of HD are known to those of skill in the medical arts. As used herein, the term. “subject” is used interchangeably with the term “patient”.

As used herein, a biological sample includes, but is not limited to: cells, tissue, body fluid (e.g. blood). The tissue may be in a subject, obtained from a subject, or may be grown in culture (e.g. from a cell line). A cell or tissue used in the invention can be a neuronal cell or other cell type including blood cells and other non-neuronal cell types. As used herein, cell samples, tissue samples, and/or blood samples, etc., may be obtained using methods well known to those of ordinary skill in the related medical arts.

As is understood by those of skill in the art, Huntington's disease (HD) includes different genotypic and phenotypic forms of HD. For example, the huntingtin gene mutation the causes HD in one subject can differ from the mutation of the huntingtin gene in another subject. Therefore, in a first subject the number of polyglutamine repeats in huntingtin protein may differ from the number of polyglutamine repeats in the huntingtin protein in a second subject. Thus, the two subjects both have HD, but have different forms of HD.

As used herein the “status” of HD means the physiological stage or clinical condition of the cell, tissue, or the subject with HD. It will be understood by those of ordinary skill that the status may reflect a number of different factors relating to the HD in the cell, tissue, or subject. These factors include, but are not limited to: the genotype of the cell, tissue, or subject, the genetic penetrance of the disorder, the length of time the disease has been manifested in the subject, and individual cell, tissue, and/or subject parameters that define the presentation of the HD in cell, tissue, or subject. The status of HD in a subject, cell, or tissue may change over time and thus the determination of a subject, cell, or tissue's status at a first time point may differ from the status of the cell, tissue, or subject's status at a second, subsequent time point.

The status of HD in a cell, tissue, or subject may be classified using general categories such as early-stage, mid-stage, or late-stage HD and the physiological manifestation of the HD may be generally classified as mild, medium, or severe, with various gradations in between. In some embodiments, stages of HD are categorized as early presymptomatic, late presymptomatic, and symptomatic which include carriers of the gene mutation, as determined by genetic testing. For example, an early-presymptomatic group may include subjects that are carriers of an HD gene mutation are less than about 30 years of age. A late-presymptomatic group may include subjects that are carriers of a HD mutation and are over 30 years of age but are presymptomatic. Symptomatic subjects may have symptoms of HD and have a higher age, for example may be over 45 years of age. The determination of the neurological status and/or stage of HD patients can be done using the Huntington's Disease Rating Scale (UHDRS). (Mov Disord. 11: 136-142, 1996).

The status of HD may also be classified in terms of cell condition, regional involvement of cells and/or tissues, etc. In some embodiments, the status of the disease means the level of pathogenesis from the disease. Thus, at early stages of HD, pathogenesis may be mild or non-detectable and at mid and late stages the pathogenesis may be more pronounced. As used herein, the term “pathogenesis” means the clinical and physiological process and effects of the disease. Thus, as HD progresses, pathogenic features of HD such as cell degeneration will increase. Molecular pathways that lead to neuronal degeneration and death are implicated as contributory to neurodegeneration in HD, including de-regulation (dysregulation) of transcription, proteosomal dysfunction, altered proteolysis of mutant huntingtin, alterations in cytoskeleton function, and abnormalities in energy metabolism and mitochondrial function. Because mutant huntingtin is expressed in every cell of the body, these abnormal pathways may exist in non-neuronal cells, such as blood cells.

The methods and kits of the invention are useful for the diagnosis for HD in cells, tissues, and subjects. As used herein, the term “diagnostic” means the initial recognition of HD in a cell, tissue, and/or subject and also means the determination of the status or stage of HD in the cell, tissue, and/or subject. For example, a diagnosis of HD in a subject using a methods of the invention may include the determination of the stage of HD, and/or pathogenic features of HD in the subject. Thus, the level of expression of one or more HD-associated genes can be used to determine the stage or status of HD in a subject. High level of expression of one or more HD biomarker genes in a sample from a subject may be correlated with advanced stage HD, with concomitant advanced pathologic features in the cells and tissues of the subject. Similarly, a lower level of expression of one or more HD biomarker genes in a sample from a subject may be correlated with a less advanced stage HD, (e.g. early presymptomatic and late presymptomatic stages) with concomitant less advanced pathologic features in the cells and tissues of the subject. Thus, the relative levels and changes in the level of expression of an HD biomarker gene of the invention provide diagnostic information about the stage and status of HD in a cell, tissue, and/or subject.

It will be understood by those of skill in the art that the expression level of some HD-associated genes may be higher in HD and some may be lower in HD when compared to a control. Thus, determination of the level of expression of a set of HD-associated genes in a biological sample may include some HD-associated genes with higher expression and some with lower expression than that found in a control sample. In some embodiments of the invention, preferred HD biomarkers are HD biomarkers with increased levels of expression in HD. Some HD biomarker molecules of the invention may be expressed at an early presymptomatic stage at a level higher than a normal expression level (control level) and the expression level may decrease (or increase) at a later presymptomatic and/or symptomatic state. It will be understood that the level of expression of some HD biomarkers of the invention may be lower at more progressive stages of HD. Expression of some HD biomarkers of the invention may increase and some may decrease at different stages of HD. Thus, in some embodiments, determination of an expression pattern of more than one HD biomarker of the invention may be used as an indication of the stage or status of HD in a cell, tissue, or subject.

Methods for identifying subjects suspected of having HD may include genetic testing, subject's family medical history, subject's medical history, or imaging technologies, such as magnetic resonance imaging (MRI). Such methods for identifying subjects suspected of having HD are well known to those of skill in the medical arts. As used herein, the phrase “suspected of having HD” means a subject believed by one of ordinary skill in the medical arts to have HD. Additionally a cell or tissue “suspected of having HD” means a cell, or tissue believed by one of ordinary skill in the medical arts to contain HD cells.

Levels of expression of an HD-associated gene are preferentially compared to controls. The control may be a predetermined value, which can take a variety of forms. It can be a single value, such as a median or mean. A control value can be established based upon comparative groups (e.g. comparative cell types), such as in cells having normal levels of expression of one or more HD-associated genes. In some embodiments, a control level may be the level found in early presymptomatic cells, tissues, and/or subjects, or the level found in late presymptomatic cells, tissues, and/or subjects. In some embodiments, a control level of expression of an HD-associated gene or biomarker may be the level found in symptomatic cells, tissues, and/or subjects. These types of control values can serve as control values for substantially similar cells that are contacted with an HD treatment compound. For example, a control for a sample comprising blood may be a control sample comprising blood, and a control for a tissue sample may be a control tissue sample. In some embodiments, a control may be the level of expression of one or more HD-associated genes in a cell that is HD-free. A control may also be the level of expression of an HD-associated gene in a wild-type cell or in a cell with a huntingtin mutation (e.g. to compare expression levels of an HD-associated gene before and after treatment).

In some embodiments of the invention, a control level of expression of an HD-associated gene is the level of expression of the HD-associated gene in a non-HD cell, tissue, and/or subject. Non-HD cells, tissues, and/or subjects are also referred to herein as “healthy” or “HD-free” cells, tissues, and/or subjects. In some embodiments, a control level of expression of an HD-associated gene may be a level of expression of an HD-associated gene from an HD cell that is compared to a subsequent level of expression of the HD-associated gene from the same tissue and/or subject. Thus, a control level may be the starting level of expression of an HD-associated gene in a cell, tissue, or subject, and this control level of expression of an HD-associated gene can be used as a baseline to monitor cells, or a tissue and/or subject over time or in response to therapy. For example, the level of expression of one or more HD-associated genes in a cell, tissue, and/or subject during and/or following HD treatment can be compared to the starting level of expression of the one or more HD-associated genes in a cell, tissue, and/or the subject prior to treatment or between treatments, or in a cell, tissue, and/or subject that was not treated. Such a comparison allows identification of pathogenic changes and/or changes in status (e.g. stage) of the HD and also permits the determination of the efficacy of the HD treatment.

Cells for HD-associated gene expression assessment and testing may be cells from subjects and/or cultured cells known to have, or suspected of having HD. Additionally, cells from subjects with HD and cells from groups without HD can be compared for the level of HD-associated gene expression. Other comparative cell types would be cells from subjects with a family history of a disease or condition and a group without such a family history. Another group of cells that can be used in the methods of the invention are cultured cells that have been treated or cells from a subject who has been treated for HD.

A predetermined control value of course, will depend upon the particular population of cells selected. For example, an apparently healthy cell population will have a different ‘normal’ range of levels of expression of HD-associated genes than will a population that is known to have HD. Accordingly, the predetermined value selected may take into account the category in which a cell type falls. Appropriate ranges and categories can be selected with no more than routine experimentation by those of ordinary skill in the art. By “abnormal level” it is meant abnormal expression of an HD-associated gene (high or low) relative to a selected control. It will also be understood that the controls for use in the invention may be, in addition to predetermined values, samples of materials tested in parallel with the experimental materials. Examples include samples from control cells or control samples that are tested in parallel with the experimental samples.

Because of the variability of the cell types in cell and tissue biopsy or sample material (e.g. a blood sample), and the variability in sensitivity of the diagnostic methods used, the sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the HD-associated nucleic acid or polypeptide for use in the invention are well known to one of ordinary skill in the art. An example of this, although not intended to be limiting, is that in some instances a sample from the biopsy or sample may be sufficient for assessment of RNA expression without amplification, but in other instances the lack of suitable cells in a small biopsy region or sample may require use of RNA conversion and/or amplification methods or other methods to enhance resolution of the nucleic acid molecules. Such methods, which allow use of limited biopsy or sample materials, are well known to those of ordinary skill in the art and include, but are not limited to: direct RNA amplification, reverse transcription of RNA to cDNA, amplification of cDNA, or the generation of radio-labeled nucleic acids.

In some embodiments, an HD nucleic acid molecule is a nucleic acid molecule that is amplified using one or more pairs of the 14 pairs (one each forward and reverse) of primer sequences provided as sequences numbered 1 through 28 in Table 2 (SEQ ID NOs:1-28). The primer pairs are identified as able to amplify, under appropriate conditions, a sequence of an HD-associated gene of the invention. One of ordinary skill will recognize that additional primers can be designed using standard methods based on the HD-associated sequences provided herein. Thus, the invention also includes the use of additional nucleic acid sequences (e.g. primers) that allow the determination of an HD-associated nucleic acid in a sample. In some embodiments, the invention relates to methods to determine the presence and/or level of expression of an HD-associated gene of the invention using the primer sequences set forth herein.

The invention involves in some embodiments, diagnosing or monitoring HD in subjects by determining the level of expression of at least 1, 2, 3, 4, 5, 6, 7, 8, 9, 10, 11, 12, 13, 14, 15, 16, 17, 18, 19, 20, or more (up to about 325) HD-associated genes. In preferred embodiments, the cell sample is a sample of blood (e.g. peripheral blood) and the expression of one or more HD-associated genes may be determined. In other embodiments, the cell sample is a tissue sample and the expression of two or more HD-associated genes may be determined.

Measurement of the level of expression of one or more HD-associated gene(s), as described herein, in a subject over time by sequential determinations permits monitoring of the disease and/or the effects of a course of treatment. For example, a sample may be obtained from a subject, tested for the presence and/or level of expression of one of the HD-associated genes and at a second, subsequent time, another sample may be obtained from the subject and similarly tested. The results of the first and second (e.g., subsequent) tests can be compared as a measure of the status or stage of HD in the subject or cells.

In some embodiments, the comparison can be used to assess the onset, progression, or regression of HD pathogenesis, or, if HD treatment was undertaken during or shortly before the interval between obtaining the samples, the effectiveness of the treatment may be evaluated by comparing the results of two or more sample measurements. In all embodiments, treatment for HD may include, but is not limited to: drug intervention and adjuvant systemic therapies. In some embodiments, treatment of HD includes administration of a histone deacetylase (HDAC) inhibitor. An HDAC inhibitor used for treatment of HD may be selected from the group consisting of short-chain fatty acids, hydroxamic acids, epoxyketone-containing cyclic tetrapeptides, non-epoxyketone-containing cyclic tetrapeptides, benzamides, depudecin ((2R,3S,4S,5E,7S,8S,9R)-2,9-Dihydroxy-3,4,7,8-diepoxyundeca-5,10-diene) and organosulfur compounds. In some embodiments, the HDAC inhibitor is a short-chain fatty acid and the short-chain fatty acid is selected from the group consisting of butyrate, phenylbutyrate and valproate. In some embodiments, the HDAC inhibitor is a hydroxamic acid and is selected from the group consisting of trichostatins including trichostatin A (TSA), suberoylanilide hydroxamic acid (SAHA) and its derivatives, oxamflatin, azelaic bishydroxamic acid (ABHA), Scriptaid, pyroxamide and propenamides. in some embodiments, the HDAC inhibitor is an epoxyketone-containing cyclic tetrapeptide and is selected from the group consisting of trapoxins, HC-toxin, Chlamydocin, Diheteropeptin, WF-3161 (cyclo(L-Leu-L-Pip-L-Aoe-D-Phe) where Pip=pipecolic acid and Aoe=2-amino-8-oxo-9,1O-epoxydecanoic acid), Cyl-1 and Cyl-2. In some embodiments, HDAC inhibitor is a non-epoxyketone-containing cyclic tetrapeptide and is selected from the group consisting of FR901228, apicidin and cyclic-hydroxamic-acid-containing peptides (CHAPs). In some embodiments of the invention, the HDAC inhibitor is a benzamide and is selected from the group consisting of MS-275 (MS-27-275), CI-994 (N-acetyldinaline), M344 (4-Dimethylamino-N-(6-hydroxycarbamoylhexyl)-benzamide) and benzamide analogs.

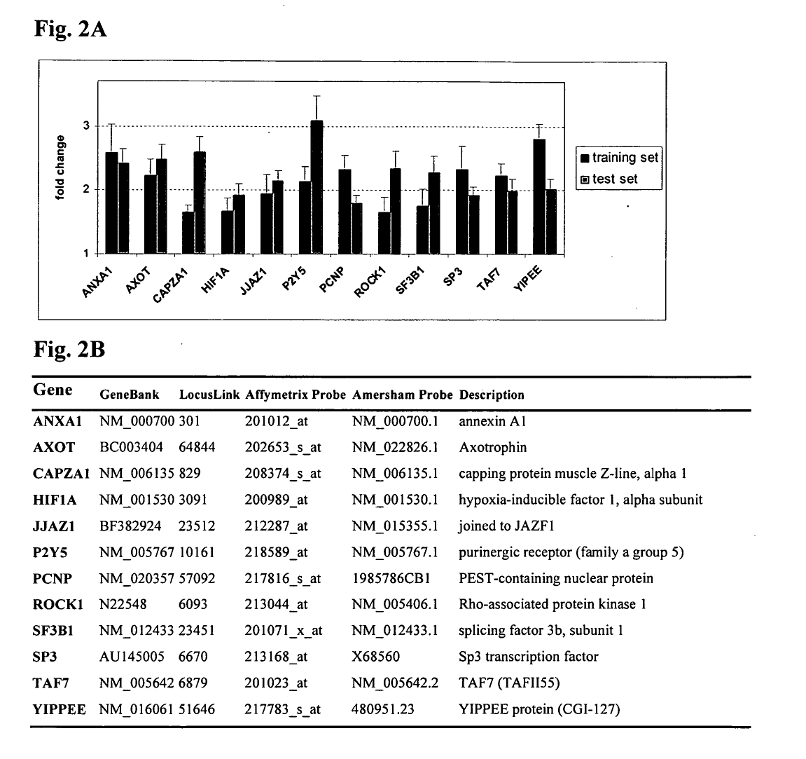

The invention involves in one aspect, HD-associated genes and the nucleic acids they encode, functional modifications and variants of the foregoing, useful fragments of the foregoing, as well as diagnostics relating thereto, and diagnostic uses thereof. In some embodiments, the HD-associated genes correspond to SEQ ID NOs: 29-40 (see FIG. 2B). Encoded polypeptides (e.g., proteins), peptides and antisera thereto are also preferred for diagnosis. In some embodiments, encoded polypeptides (e.g. proteins), peptides, and antisera thereto.

Some of the genes identified herein as HD-associated genes are newly identified as being associated with HD. All of the HD-associated sequences described herein are deposited in databases such as GenBank. The use of HD-associated sequences in diagnostic assays for HD in blood cells is novel, as is the use of sets of at least two or more of the sequences for diagnostic assays for HD in blood and tissue samples in HD diagnostic assays and kits.

The isolation and identification of HD-associated genes and polypeptides also permits the artisan to diagnose a disorder characterized by expression of HD-associated polypeptides. The methods related to HD-associated polypeptide expression involve determining expression of one or more HD-associated nucleic acids, and/or encoded HD-associated polypeptides and/or peptides derived therefrom and comparing the expression with that in an HD-free subject. Such determinations can be carried out via any standard nucleic acid determination assay, including the polymerase chain reaction, or assaying with labeled hybridization probes. Such hybridization methods include, but are not limited to, microarray techniques.

The invention also includes methods to monitor the onset, progression, or regression of HD in a subject by, for example, obtaining samples at sequential times from a subject and assaying such samples for the presence and/or absence of an antigenic response that is a marker of the condition. A subject may be suspected of having HD or may be believed not to have HD and in the latter case, the sample may serve as a normal baseline level for comparison with subsequent samples.

Onset of a condition is the initiation of the changes associated with the condition in a subject. Such changes may be evidenced by physiological symptoms, or may be clinically asymptomatic. For example, the onset of HD may be followed by a period during which there may be HD-associated pathogenic changes in the subject, even though clinical symptoms may not be evident at that time. The progression of a condition follows onset and is the advancement of the pathogenic (e.g. physiological) elements of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

A marker for HD may be the presence or abnormal amount of transcription or translation of an HD-associated gene and/or specific binding of an HD-associated polypeptide with an antibody. Onset of an HD condition may be indicated by the appearance of such a marker(s) in a subject's samples where there was no such marker(s) determined previously. For example, if marker(s) for HD are determined not to be present in a first sample from a subject, and HD marker(s) are determined to be present in a second or subsequent sample from the subject, it may indicate the onset of HD.

Progression and regression of HD may be generally indicated by the increase or decrease, respectively, of marker(s) (that are identified as being upregulated in HD) in a subject's samples over time. For example, if marker(s) for HD are determined to be present in a first sample from a subject and additional marker(s) or a different amount of the initial marker(s) for HD are determined to be present in a second or subsequent sample from the subject, it may indicate the progression of HD and/or additional HD pathogenesis. Regression of HD and/or a reduction in pathogenesis may be indicated by finding that marker(s) determined to be present in a sample from a subject are not determined to be found, or found at different amounts in a second or subsequent sample from the subject.

The progression and regression of HD may also be indicated based on characteristics of the HD-associated polypeptides determined in the subject. For example, some HD-associated polypeptides may be abnormally expressed at specific stages of HD (e.g. early-stage HD-associated polypeptides; mid-stage HD-associated polypeptides; and late-stage HD-associated polypeptides). Another example, although not intended to be limiting, is that HD-associated polypeptides may be differentially expressed in different tissues, e.g. blood versus brain, thereby allowing the stage and/or diagnostic level of the disease to be established, based on the identification of selected HD-associated polypeptides in a subject sample.

Different types of HD, such as HD in a cell or subject that is characterized by a higher or lower number of huntingtin polyglutamine repeats than another HD cell or subject, may have different levels of expression of HD-associated genes and may express different HD-associated polypeptides and the encoding nucleic acid molecules thereof. Different forms of HD may also have different spatial or temporal expression patterns of HD-associated genes of the invention. Such variations may allow HD-specific diagnosis and subsequent treatment tailored to the patient's specific condition. These HD-specific diagnoses may also be based on the variations in HD-associated gene expression that are useful to determine staging of the HD in a cell or subject.

The invention includes kits for assaying the level and or presence of expression of one or more HD-associated genes. An example of such a kit may include methods for determining the level of transcription or translation of one, two, or more of the HD-associated genes of the invention. A kit of the invention may include nucleic acid or protein microarrays with HD-associated nucleic acids or the polypeptides they encode. Kits may include materials for use in standard techniques of microarray technology to assess expression of the HD-associated genes of the invention.

In some embodiments, a kit may include a PCR components, e.g. primers, solutions, polymerase, etc for amplifying mRNA from a sample or subject. A kit may include one or more antibodies to one or more of the HD-associated polypeptides of the invention along with components useful for use of the antibodies to determine expression of one or more HD-associated genes in a cell, tissue or subject. A sample may be processed using procedures well known to those of skill in the art, to assess whether specific binding occurred between the antibodies and HD-associated polypeptides in the subject's tissue or blood sample or another cell sample. For example, procedures may include, but are not limited to, contact with a secondary antibody, or other method that indicates the presence of specific binding between polypeptides in the cell or sample and an antibody that specifically binds to an HD-associated polypeptide. Kits of the invention may include primary antibodies, secondary antibodies, dishes, vessels, and solutions useful for antibody staining.

The foregoing kits can include instructions or other printed material on how to use the various components of the kits for diagnostic purposes.

Nucleic acid molecules, and fragments thereof of the invention can be used to produce fusion proteins for generating antibodies or determining binding of the polypeptide fragments, or for generating immunoassay components. Likewise, nucleic acids and fragments thereof of the invention can be employed to produce nonfused fragments of the HD-associated polypeptides, useful, for example, in the preparation of antibodies, and in immunoassays. Preferred fragments encoded by a nucleic acid of the invention or fragment thereof, are antigenic polypeptides, which are recognized by agents that specifically bind to HD-associated polypeptides. As used herein, HD-associated antibodies, are antibodies that specifically bind to HD-associated polypeptides.

The invention also permits the construction of HD-associated polypeptide gene “knock-outs” or “knock-ins” in cells and in animals, providing materials for studying certain aspects of HD and HD-associated gene expression in cells and tissues by regulating the expression of HD-associated genes. For example, a knock-in mouse may be constructed and examined for clinical parallels between the model and an HD-affected mouse with upregulated expression of an HD-associated gene, which may be useful to alter the pathogenesis of the HD. Such a cellular or animal model may also be useful for assessing treatment strategies for HD. Alternative types of animal models for HD may be developed based on the invention. Altering the expression of an HD-associated gene in an animal may provide a model in which to test treatments, and assess the etiology and pathogenesis of HD.

The invention also provides isolated polypeptides (including whole proteins and partial proteins) encoded by the foregoing HD-associated nucleic acids. Such polypeptides are useful, for example, alone or as fusion proteins to generate antibodies, and as components of an immunoassay or diagnostic assay. HD-associated polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short HD-associated polypeptides can be synthesized chemically using well-established methods of peptide synthesis.

Fragments of a polypeptide preferably are those fragments that retain a distinct functional capability of the polypeptide. Functional capabilities that can be retained in a fragment of a polypeptide include interaction with antibodies (e.g. antigenic fragments), interaction with other polypeptides or fragments thereof, selective binding of nucleic acids or proteins, and enzymatic activity.

The skilled artisan will also realize that conservative amino acid substitutions may be made in HD-associated polypeptides to provide functionally equivalent variants, or homologs of the foregoing polypeptides, i.e., the variants retain the functional capabilities of the HD-associated polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Exemplary functionally equivalent variants or homologs of the HD-associated polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a polypeptide is an HD-associated polypeptide, one can make conservative amino acid substitutions to the amino acid sequence of the polypeptide, and still have the polypeptide retain its specific antibody-binding characteristics.

Conservative amino-acid substitutions in the amino acid sequence of HD-associated polypeptides to produce functionally equivalent variants of HD-associated polypeptides typically are made by alteration of a nucleic acid encoding an HD-associated polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding an HD-associated polypeptide. Where amino acid substitutions are made to a small fragment of an HD-associated polypeptide, the substitutions can be made by directly synthesizing the peptide. The activity of functionally equivalent fragments of HD-associated polypeptides can be tested by cloning the gene encoding the altered HD-associated polypeptide into a bacterial or mammalian expression vector, introducing the vector into an appropriate host cell, expressing the altered polypeptide, and testing for a functional capability of the HD-associated polypeptides. Peptides that are chemically synthesized can be tested directly for function, e.g., for binding to antisera recognizing associated antigens.

The invention also makes it possible to isolate proteins that specifically bind to HD-associated polypeptides as disclosed herein, including antibodies and cellular binding partners of the HD-associated polypeptides. Additional uses are described further herein. Thus, the invention also involves agents such as polypeptides that bind to HD-associated polypeptides. Such binding agents can be used, for example, in screening assays to detect the presence or absence of HD-associated polypeptides and complexes of HD-associated polypeptides and their binding partners and in purification protocols to isolate HD-associated polypeptides and complexes of HD-associated polypeptides and their binding partners. Such agents also may be used to inhibit the native activity of the HD-associated polypeptides, for example, by binding to such polypeptides. The invention, therefore, embraces peptide-binding agents that, for example, can be antibodies or fragments of antibodies having the ability to selectively bind to HD-associated polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc' and Fc regions, for example, are effectors of the complement cascade but are not involved in antigen binding. An antibody from which the pFc' region has been enzymatically cleaved, or which has been produced without the pFc' region, designated an F(ab')₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc' regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab')₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab')₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to HD-associated polypeptides, and complexes of both HD-associated polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the HD-associated polypeptide. This process can be repeated through several cycles of reselection of phage that bind to the HD-associated polypeptide. Repeated rounds lead to enrichment of phage bearing particular sequences. DNA sequence analysis can be conducted to identify the sequences of the expressed polypeptides. The minimal linear portion of the sequence that binds to the HD-associated polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the HD-associated polypeptides.

Thus, the HD-associated polypeptides of the invention, including fragments thereof, can be used to screen peptide libraries, including phage display libraries, to identify and select peptide binding partners of the HD-associated polypeptides of the invention. Such molecules can be used, as described, for screening assays, for purification protocols, for interfering directly with the functioning of HD-associated polypeptides and for other purposes that will be apparent to those of ordinary skill in the art. For example, isolated HD-associated polypeptides can be attached to a substrate (e.g., chromatographic media, such as polystyrene beads, or a filter), and then a solution suspected of containing the binding partner can be applied to the substrate. If a binding partner that can interact with HD-associated polypeptides is present in the solution, then it will bind to the substrate-bound HD-associated polypeptide. The binding partner then may be isolated.

As detailed herein, the foregoing antibodies and other binding molecules may be used for example, to identify tissues expressing protein or to purify protein. Antibodies also may be coupled to specific diagnostic labeling agents for imaging of cells and tissues that express HD-associated polypeptides or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium.

The invention further includes nucleic acid or protein microarrays with HD-associated peptides or nucleic acids encoding such polypeptides. In this aspect of the invention, standard techniques of microarray technology are utilized to assess expression of the HD-associated genes—e.g. to identify HD-associated nucleic acids and/or polypeptides. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485): 1760-1763, 2000. Nucleic acid arrays, particularly arrays that bind HD-associated peptides, also can be used for diagnostic applications, such as for identifying subjects that have a condition characterized by HD-associated gene expression. Non-limiting examples of array methods that can be used in the methods of the invention are provided in the Examples section.

### EXAMPLES

Introduction Huntington's disease (HD) is an autosomal dominant neurodegenerative disorder characterized clinically by progressive motor impairment, cognitive decline and various psychiatric symptoms with the typical age of onset in the third to fifth decades. The disorder is caused by the expansion of an unstable CAG triplet repeat. Postmortem changes in HD brains are widespread and include neuronal loss and gliosis, particularly in the cortex and the striatum (Vonsattel, J. P. et al. J. Neuropathol. Exp. Neurol. 57: 369-384, 1998). Transcriptional deregulation and loss of function of transcriptional co-activator proteins have been implicated in HD pathogenesis (Harjes, P. et al., Trends Biochem. Sci. 28: 425-433, 2003; Sugars, K. L. et al., Trends in Genetics 19: 233-238, 2003). Mutant huntingtin has been shown to specifically disrupt activator-dependent transcription in early stages of HD pathogenesis (Dunah, A. W. et al., Science 296: 2238-2243, 2002). Numerous microarray studies showed alterations in mRNA levels of a large number of genes in the brain of HD mice, suggesting that huntingtin may interfere with transcriptional mechanisms common to many genes (Luthi-Carter, R. et al., Hum. Mol. Genet. 9: 1259-1271, 2000; Luthi-Carter, R., et al., Hum. Mol. Genet. 11: 1911-1926, 2002). In addition, studies in cell culture, yeast, and Drosophila models of polyglutamine disease have indicated that histone deacetylase (HDAC) inhibitors might provide a useful class of agents to ameliorate the transcriptional changes in HD (Steffan, J. S. et al., Nature 413: 739-743, 2001; McCampbell, A. et al., Proc. Natl. Acad. Sci. USA 98: 15179-15184, 2001; Hughes, R. E. et al., Proc. Natl. Acad. Sci. USA 98: 13201-13206, 2001; Hockly, E. et al., Proc. Natl. Acad. Sci. USA 100: 2041-2046, 2003; Ferrante, R. J. et al., J. Neurosci. 23: 9418-9427, 2003).

Because mutant huntingtin is ubiquitously distributed and appears to affect widely expressed transcription factors, it has been postulated that transcriptional impairments in HD may exist in tissues outside of CNS. For example, significant alterations in MRNA expression have been detected in skeletal muscle of HD transgenic mice (Luthi-Carter, R., et al., Hum. Mol. Genet. 11: 1911-1926, 2002). Because mutant huntingtin is expressed in every cell of the body, these abnormal pathways may exist in non-neuronal cells, such as blood cells. Dividing blood cells do not degenerate because of their short half-life compared to post-mitotic neurons (reviewed in Ross, C. A., Cell, 118: 1-138, 2004). We investigated whether other tissues such as peripheral blood could be used to analyze changes in gene expression in patients with HD.

Previous work indicated that lymphoblasts derived from HD patients showed increased stress-induced apoptotic cell death (Sawa, A. et al., Nat. Med. 5: 1194-1198, 1999). Furthermore, both normal and mutant huntingtin have been found in peripheral blood of HD patients (Ide, K. et al., Biochem. Biophys. Res. Commun. 209: 1119-1125, 1995). Thus, we hypothesized that analysis of blood cells could also reveal patterns of differential gene expression that could serve as indicators of normal and abnormal biological processes in HD. Although the genetic mutation in HD serves as a definitive trait marker, differential gene expression in blood could serve as a state marker of the disease. Such biomarkers could be especially valuable in presymptomatic carriers of the HD mutation to provide an objective measure of disease state. For example, neurons are at risk several years before motor symptoms of the disease occur, but a definitive diagnosis can only be made in the presence of unequivocal motor signs of HD. Such markers are also much needed in clinical trials of symptomatic patients, which currently rely on relatively insensitive clinical measures.

We utilized oligonucleotide microarrays to analyze global changes in mRNA expression in blood samples of HD patients compared to normal controls. In addition, we analyzed peripheral blood samples from patients who were on sodium phenylbutyrate, an HDAC inhibitor, as part of a Phase I dose-finding study. Because histone acetylation and deacetylation play an important role in regulation of gene transcription, we investigated whether treatment of HD patients with sodium phenylbutyrate would affect gene expression in patients' blood. The results indicated that a subset of genes altered in peripheral blood may correlate with HD progression and response to treatment.

Methods

Isolation of Blood and Brain Samples

Peripheral blood samples were collected from 62 HD subjects and 53 age- and gender-matched healthy controls. The HD group included a cohort of 9 early asymptomatic carriers of the gene mutation, as determined by genetic testing, consisting of 5 females and 4 males with the average age of 22.5±2.6 years. An additional group of 21 late presymptomatic carriers of the HD mutation was also included in the study. Of these, 11 were females and 10 were males with the average age of 39±6.1. An additional 32 patients were symptomatic, 17 females and 15 males, with the average age of 49.6±5.9. The determination of the neurological status of the HD patients utilized the Huntington's Disease Rating Scale (UHDRS) performed by an experienced HD neurologist. All of the symptomatic patients were in Stage I or II of disease, as determined by the total functional capacity scores (TFC 7-12). All samples were obtained in accordance with the Institutional Review Boards and participants gave written informed consent. Blood samples were also obtained from 12 symptomatic patients (7 males and 5 females) participating in a dose-finding study of the HDAC inhibitor, sodium phenylbutyrate (SPB 11, Scandinavian Designs, Sellersville, Pa.) at Oregon Health and Science University. The dosage of sodium phenylbutyrate in these subjects ranged from 12 to 18 grams per day administered in 3 doses. The blood samples were collected prior to treatment and at the end of week 4 of treatment. There were no consistent differences noted in total and differential cell blood counts between control and HD patient blood or as a result of phenylbutyrate treatments. In addition, gene expression levels of known markers of lymphocytes (interferon induced genes), neutrophils (lactotransferrin), and reticulocytes (hemoglobin, delta) exhibited no systematic differences between the HD and control samples. Any and all concomitant medications were held stable throughout the sodium phenylbutyrate treatment period. For experiments involving human brain, caudate nuclei from 5 HD postmortem frozen brain samples (Vonsattel grades 0-2) and 4 age- and gender-matched controls with similar postmortem intervals were analyzed.

RNA Isolation and Gene Profiling

Total RNA from blood was extracted using PAXgene™ Blood RNA Kit (Qiagen Inc., Valencia, Calif.), and RNeasy® Lipid Tissue Kit (Qiagen) was used for isolation of total RNA from striatum according to manufacturer's protocol. All samples were treated with RNase-Free DNase Set (Qiagen). The quality of total RNA was analyzed using the RNA 6000 Nano LabChip kit on a 2100 Bioanalyzer (Agilent Technologies, Palo Alto, Calif.). Microarray analysis was performed using U133A GeneChips (Affymetrix, Santa Clara, Calif.) and CodeLink Uniset Human I and II Bioarrays (Amersham Biosciences, Piscataway, N.J.). The U133A GeneChip contains 22,283 25-mer probe sets and the Uniset Human I and II Bioarrays contain 20,289 30-mer probes. Approximately 11,000 genes with unique LocusLink IDs are common to both platforms. The RNA for both platforms was processed using a modified Amersham CodeLink protocol (CodeLink Expression Bioarrays, Automated target preparation, User guide. Amersham Biosciences). Briefly, 4 μg of high quality total RNA were reverse transcribed (Invitrogen, Carlsbad, Calif.), then cleaned using the QIAquick Purification kit (Qiagen) and used as a template for In Vitro Transcription (IVT) utilizing Ambion's T7 MEGA script reagents (Ambion, Inc., Austin, Tex.) and Biotin-11-UTP (PerkinElmer/NEN, Boston, Mass.). Resulting biotin-labeled cRNA was recovered and purified with RNeasy® kit (Qiagen), then hybridized to the chips, and fluorescently tagged and scanned according to manufacturer's protocol. Usual quality measures and normalization for both the Affymetrix GeneChip (3′/5′ ratios and trimmed mean normalization) and the CodeLink microarrays (detection thresholds and median normalization) were used in the experiments. All arrays were run in the same core facility.

QRT-PCR Analysis

Reverse transcription (RT) was performed using total RNA isolated from blood or postmortem brain and processed with SuperScript™ First-Strand Synthesis System for RT-PCR according to the manufacturer's protocol (Invitrogen). QRT-PCR was performed with the SYBR Green method using the MyiQ Single-Color Real-Time PCR Detection System (BioRad, Hercules, Calif.). Primers were designed with the Primer3 program (rodo.wi.mit.edu/cgi-bin/primer3/primer3 www.cpi) and the sequences (F stands for forward and R for reverse primer) were as follows in Table 2.

TABLE 2
Primer sequences.

| | |
|---|---|
| ANXA1-F: | |
| GAGCCCCTATCCTACCTTCA, | (SEQ ID NO:1) |
| ANXA1-R: | |
| GGTTGCTTCATCCACACCT, | (SEQ ID NO:2) |
| AXO-F: | |
| ACATCAGATTCGGCTCAAGG, | (SEQ ID NO:3) |
| AXO-R: | |
| GGACTGCAAACCGGAATAAG, | (SEQ ID NO:4) |
| CAPZA1-F: | |
| TTGGAGGGCAAAGGAAGT, | (SEQ ID NO:5) |
| CAPZA1-R: | |
| GCAGGGAATGTGGTTCAAGT, | (SEQ ID NO:6) |
| HIF1A-F: | |
| CGCAAGTCCTCAAAGCACA, | (SEQ ID NO:7) |
| HIF1A-R: | |
| TCAGTGGTGGCAGTGGTAGT, | (SEQ ID NO:8) |
| JJAZ1-F: | |
| GATGGGGAAGTAGAACAGCA, | (SEQ ID NO:9) |
| JJAZ1-R: | |
| CGGAGAGGTAAGCAGGTATCA, | (SEQ ID NO:10) |
| P2Y5-F: | |
| TTGGACGTGCCTTTACGA, | (SEQ ID NO:11) |
| P2Y5-R: | |
| TGCTGAACATGCACCCATAC, | (SEQ ID NO:12) |
| PCNP-F: | |
| CTGTTCCAACTCTTGCTCCA, | (SEQ ID NO:13) |
| PCNP-R: | |
| GGCATTTCCTCTGGTTCACT, | (SEQ ID NO:14) |
| ROCK1-F: | |
| TGAGGTTAGGGCGAAATGGT, | (SEQ ID NO:15) |
| ROCK1-R: | |
| AATCGGGTACAACTGGTGCT, | (SEQ ID NO:16) |
| SF3B1-F: | |
| CTTATGGGCTGTGCCATCTT, | (SEQ ID NO:17) |
| SF3B1-R: | |
| GTCCGAACTTTCTGCTGCTC, | (SEQ ID NO:18) |
| SP3-F: | |
| CCTTACTTGCCTCTGGAACA, | (SEQ ID NO:19) |
| SP3-R: | |
| CTCCCTGAACCTGGACTTGA, | (SEQ ID NO:20) |
| TAF7-F: | |
| CGGGAGAGTTTGTGAGTTGA, | (SEQ ID NO:21) |
| TAF7-R: | |
| AGCTAGGGAACAGGAAAGCA, | (SEQ ID NO:22) |
| YIPPEE-F: | |
| GAGAGTGAGGGCTTTGAGGA, | (SEQ ID NO:23) |
| YIPPEE-R: | |
| GGAGACCTGGGAAAAGATGG, | (SEQ ID NO:24) |
| ACTIN-F: | |
| TCCCTGGAGAAGAGCTACGA, | (SEQ ID NO:25) |
| ACTIN-R: | |
| AGGAAGGAAGGCTGGAAGAG, | (SEQ ID NO:26) |
| 28S-F: | |
| AAACTCTGGTGGAGGTCCGT, | (SEQ ID NO:27) |
| 28S-R: | |
| CTTACCAAAAGTGGCCCACTA. | (SEQ ID NO:28) |

Initial analysis was performed using the iCycler system software (Biorad). Relative gene expressions were calculated using the 2^{-ΔΔCt} method (Livak, K. J. et al., Methods 25: 402-408, 2001). β-actin was used as an internal control for blood samples and 28S rRNA as an internal control for striatum samples.

Statistical Analysis

Data analysis was performed using the Affymetrix MAS 5.0 software, the Amersham CodeLink software, Excel, Access, S-plus 6.1 and SpotFire DecisionSite for Functional Genomics 8.0. Briefly, Affymetrix MAS 5.0 and Amersham CodeLink software were used for acquisition and calculation of signal values on Affymetrix and Amersham microarrays, respectively. Calculation of maximal value of expression, two-sided t-test and ratio of change were performed by Excel, while Access was used for filtering of the statistically significant genes on both microarray platforms. Principal component analysis (PCA) of microarray and QRT-PCR results was done with S-Plus 6.1. Normalization of microarray data and hierarchical clustering were performed using SpotFire DecisionSite for Functional Genomics 8.0.

The genes with low expression levels have lower copy numbers of mRNA and are most susceptible to technical noise. Therefore, in the analysis of the Affymetrix microarray data only genes with MAS 5.0 “signal” intensity in at least one sample above the “target intensity” of 100 were considered for further analysis. For Amersham microarrays, a similar filter was applied using the CodeLink median expression level of 1 as the filtering criterion. We further filtered the data using a two-sided Students T-test with P<0.0005 and expression ratio of average HD/average healthy control>1.8 or <0.6 as cut-off values. The lists of most significantly changed genes from both platforms were cross-referenced and duplicate entries excluded. Hierarchical clustering on normalized (median-polished) samples using cosine correlation with complete linkage was performed on the pool of all samples from both platforms to determine the gene clustering and to better visualize differences in expression profiles between the HD patients and healthy control subjects (SpotFire DecisionSite for Functional Genomics 8.0).

For QRT-PCR data the average fold change=2^{-(average ΔΔCt)} was computed using the average difference in the ΔCt between the genes and internal controls. The error bars on the column plots were determined by the Standard Error of Mean difference (SEM) of the ΔCt values so that the top of the error bar corresponds to (average fold change)×(2^{SEM}31 1). We also performed principal component analysis (PCA) with S-Plus 6.1 (Insightful) using the ΔCt values for each gene of the 12-gene marker set, compared to β-actin as control. To account for possible technical variability in different QRT-PCR experiments all the Δct values were normalized to the median of matched control samples used in each experiment. We calculated the principal components on the training set and then used the same principal components to analyze a test set. We plotted the first two principal components as they captured the most variation in the original data.

Results

Microarray Analysis of Global Gene Expression Changes in Blood of HD Patients

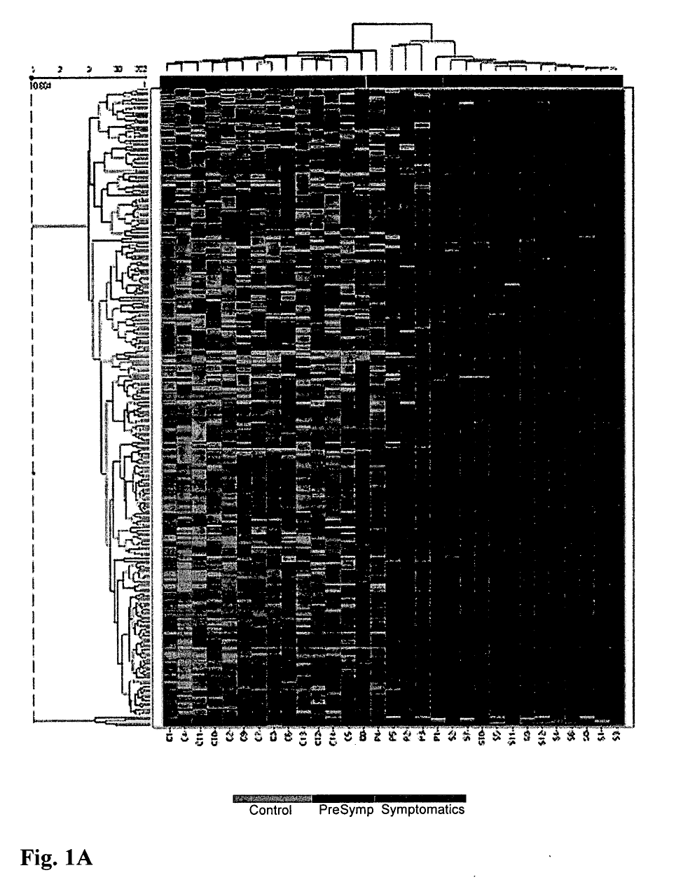

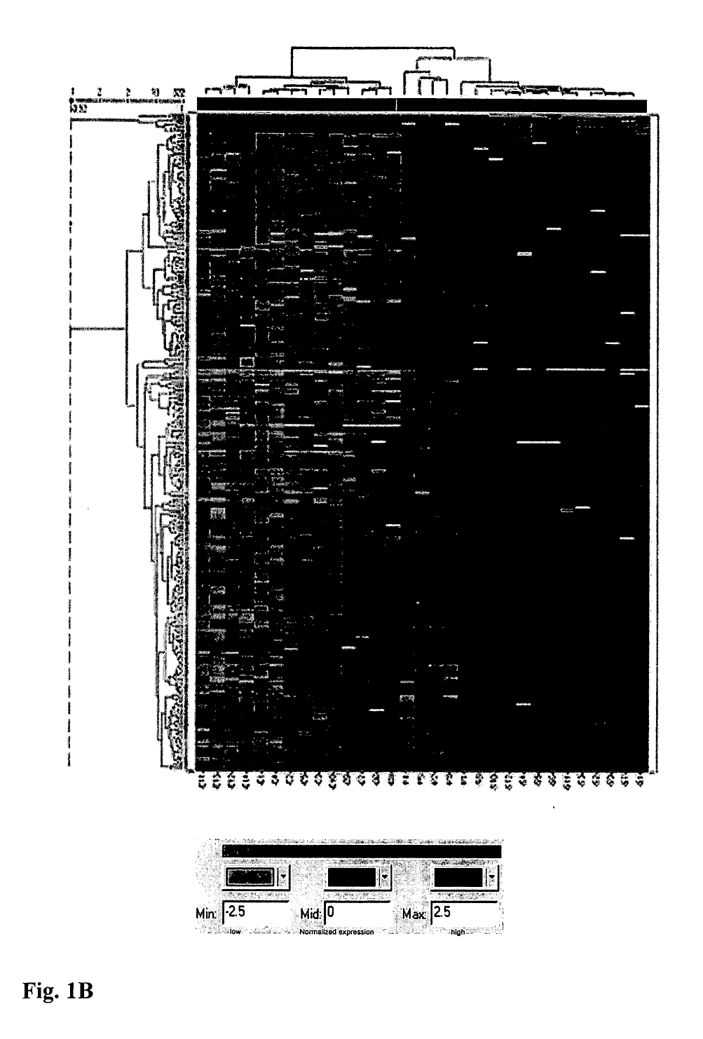

Using Affymetrix GeneChip U133A and Amersham CodeLink arrays, global gene expression changes were analyzed in blood samples from 17 HD affected subjects (12 symptomatic and 5 late presymptomatic carriers of the HD mutation) and 14 healthy, age-and gender-matched control subjects. The Affymetrix platform identified 773 significantly changed genes (P<0.0005, average HD/average healthy controls expression ratio>1.8 or <0.6, expression level>100). Using Amersham CodeLink arrays for high-throughput validation of the Affymetrix data, we selected 322 genes that were also significantly changed on the Amersham arrays (P<0.0005, ratio>1.8 or <0.6, expression level>1). Cluster analysis of the Affymetrix and Amersham expression levels of the significantly changed genes showed that the majority of the genes were upregulated in HD as compared to control blood samples (FIG. 1). Interestingly, 4 of the significantly changed genes were upregulated on Affymetrix and downregulated on Amersham platform. Our initial investigation suggested that probes on different platforms may hybridize to different splice variants of each gene but further experiments will be required to confirm this observation.

Genes that had significant changes in expression belong to many different functional groups such as transcription/RNA processing, signaling, ubiquitin/proteasome, and vesicle trafficking. Those involved in transcription include transcriptional co-activators such as TAF7, splicing factors such as SF3B 1, transcriptional regulators such as retinoblastoma 1, and zinc finger proteins. Other differentially expressed genes include those involved in signal transduction such as purinergic receptor P2Y5, members of the tumor necrosis factor receptor superfamily, and ubiquitin/proteasome-associated proteins such as ubiquitin-specific protease USP 15. Genes involved in vesicle trafficking such as proteoglycan 1 were also significantly changed.

Selection of Biomarker Genes

In order to identify a subset of genes that could usefully classify HD patients and healthy controls, 322 genes (see Table 1) were ranked according to their P values, highest fold change, highest expression levels and consistency of fold change in each individual HD sample compared to its age and gender matched control. Probe sequences that corresponded to duplicates of the same genes, as well as probes for unknown or hypothetical proteins were removed. In the case of duplicate entries, probes with most statistically significant changes were used in further analysis. Using such criteria we selected top 30 candidate genes that were further tested by QRT-PCR using blood samples from 11 HD affected subjects (6 new symptomatic patients and 5 late presymptomatic carriers of the HD mutation that were used for microarrays) and 5 new, age- and gender-matched controls. Using this approach, we identified 12 genes that exhibited most statistically significant changes between the HD and control groups (FIGS. 2A, 2B). To confirm the ability of the selected marker set to discriminate between healthy controls and HD subjects we used a test set consisting of a new cohort of 30 HD subjects (14 symptomatic patients and 16 late presymptomatic carriers of the HD mutation) and 25 matched controls. We found a statistically significant increase in the expression of the biomarker genes in HD subjects when compared to controls (FIG. 2A).

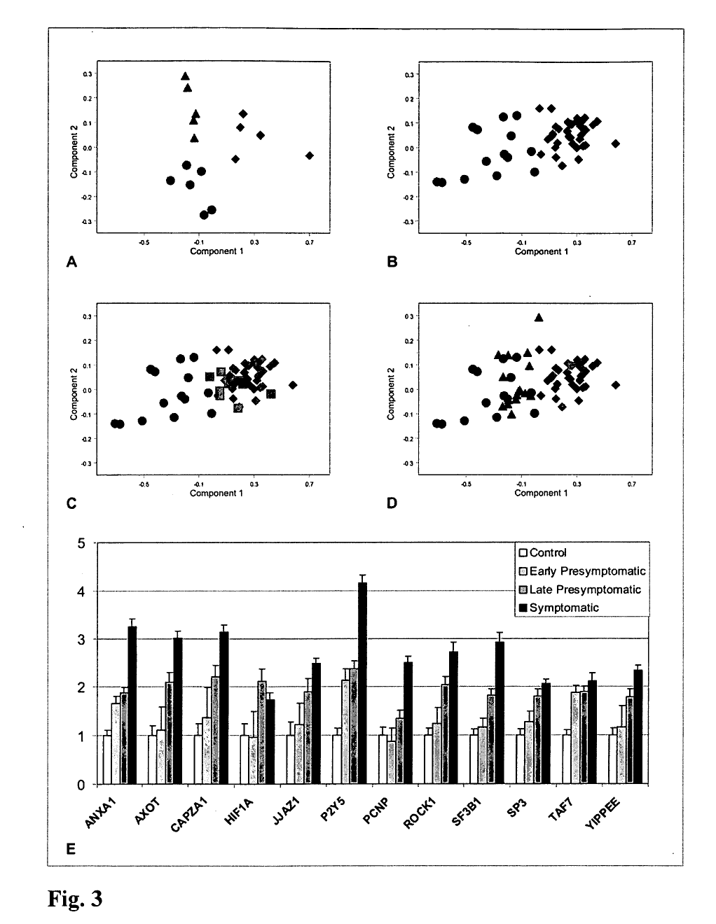

Next, principal component analysis (PCA) was performed to better visualize the differences in expression of the 12 genes in relation to the stage of disease. Such analysis of the training set confirmed that the 12 genes were able to clearly separate the HD subjects and controls in the training sets using Affymetrix, Amersham, and as shown in FIG. 3A) QRT-PCR data. In addition, PCA analysis showed clear differences in mRNA expression between symptomatic and late presymptomatic HD subjects with the presymptomatics clustering between the symptomatic patients and control subjects. Although there were some differences between the Affymetrix and Amersham analysis, namely in the classification of the presymptomatic and control samples, the concordance of the two platforms was greater than 0.7 for each of the 12 genes as determined by Pearson correlation, with P<0.00005.

To further validate the biomarker genes using the independent test set, the QRT-PCR data was also projected onto the first two principal components identified for the training set. The PCA of the HD and control samples in the test set confirmed total separation of the two groups (FIG. 3B). Late presymptomatic carriers of the HD mutation (average age 39±6.1) clustered predominantly with the symptomatic patients although some overlap with control subjects existed (FIG. 3C). Next, expression of the 12 marker genes was examined in a new cohort of 9 early presymptomatic HD subjects (average age 22.5±2.6) and 9 matched controls. Interestingly, these subjects clustered between symptomatic HD and controls but overlapped considerably with the control group (FIG. 3D). Of the 12 genes tested only 3 genes showed significant upregulation in early presymptomatic subjects confirming that expression profiles in early stages of HD more resemble controls (FIG. 3E). Moreover, expression of the marker genes progressively increased when HD progressed from early presymptomatic to late presymptomatic and symptomatic stage (FIG. 3E).

These results confirmed that the expression of the 12-gene marker set could clearly distinguish diseased blood samples from controls and may also discriminate stages of disease progression.

Biomarker Gene Changes in Response to Treatment in HD

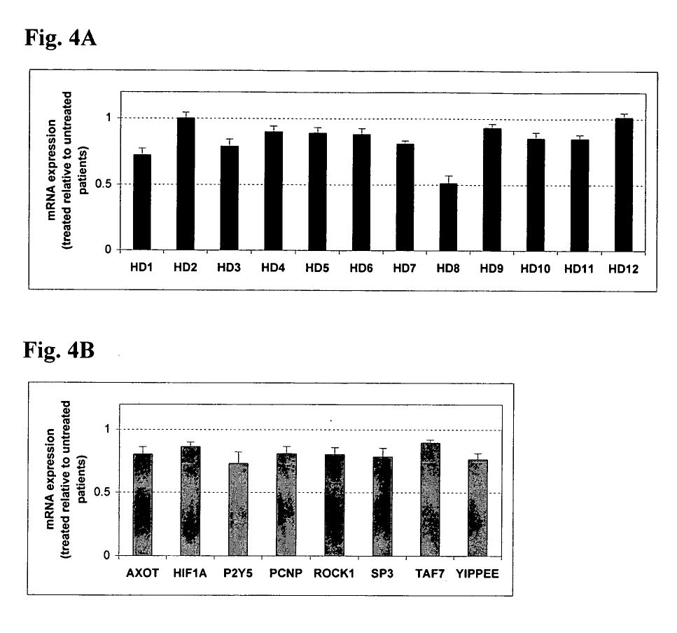

We examined whether the expression of the 12 marker genes could be modified by treatment with sodium phenylbutyrate, an HDAC inhibitor, conducted as part of a Phase I dose-finding study. Sodium phenylbutyrate has been shown to exert significant neuroprotective effects in transgenic mouse models of HD (Gardian, G. et al., J. Biol. Chem. 280: 556-563, 2005). As an initial step for assessing the feasibility of phenybutyrate administration in HD patients, a dose-finding study was performed. Blood samples were collected from 12 HD patients prior to treatment and at week four of treatment with phenylbutyrate. Using QRT-PCR, we found a small but statistically significant decrease in expression of the 12-gene marker set in 10 out of 12 patients after 4 weeks of treatment (FIG. 4A). When the marker genes were examined individually, 8 out of 12 genes were significantly decreased in response to 4 weeks of treatment with phenylbutyrate (FIG. 4B).

β-actin controls used in these experiments did not exhibit any systematic changes in expression between the untreated and treated groups of patients. There were no significant symptomatic effects of phenylbutyrate in any of the subjects as expected in a dose-finding study not powered to detect alterations in symptoms or progression.

Together, these results indicated that the selected biomarker set of 12 genes may be used to monitor early responses to treatment in patients with Huntington's disease.

Expression of Biomarker Genes in Human HD Brain Tissue

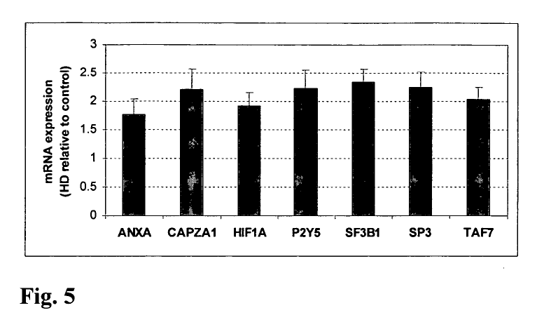

In order to examine whether the changes in mRNAs observed in HD patient blood correlated with MRNA changes in brain, the expression of the 12 genes was analyzed in human HD brain tissues using QRT-PCR in caudate nuclei isolated from 5 postmortem HD (Vonsattel grades 0-2) and 4 control brains. Among the 12 genes tested, 7 exhibited significant upregulation in HD brain samples compared to controls whereas the other 5 were not significantly altered (FIG. 5). Although the mechanisms of differential gene expression between the blood and brain tissues are not fully elucidated, we determined that alterations of gene expression in HD blood at least in part correlate with changes in gene expression in HD brain, indicating that mutant huntingtin may affect similar targets in these tissues. These results indicated that blood markers may provide a more accessible window through which it is possible to monitor the underlying pathogenic process in HD.

Discussion

Our results demonstrated dramatic alterations in gene expression in the peripheral blood of Huntington's disease patients. Although huntingtin is an ubiquitously expressed protein, this was an unexpected finding considering that no primary pathology outside of the CNS has been identified in HD. In order to analyze the observed changes in mRNAs in more detail, we focused on a subset of 12 genes that were able to clearly distinguish normal controls and patients with HD. Most importantly, the expression of these marker genes was altered in relation to disease progression from early presymptomatic to late presymptomatic and symptomatic stage of HD. Gene expression in younger carriers of the HD mutation (average age 22 years) resembled the expression patterns observed in control subjects whereas older presymptomatic subjects (average age 39 years) became more similar to symptomatic HD patients (average age 49 years). These findings indicated that a subset of mRNAs isolated from blood could be useful in monitoring the progression of HD.

Studies in cell culture, yeast, and Drosophila models of polyglutamine disease indicated that HDAC inhibitors might provide a useful class of agents to ameliorate the process of neurodegeneration in HD (Steffan, J. S. et al., Nature 413: 739-743, 2001; McCampbell, A. et al., Proc. Natl. Acad. Sci. USA 98: 15179-15184, 2001; Hughes, R. E. et al., Proc. Natl. Acad. Sci. USA 98: 13201-13206, 2001; Hockly, E. et al., Proc. Natl. Acad. Sci. USA 100: 2041-2046, 2003; Ferrante, R. J. et al., J. Neurosci. 23: 9418-9427, 2003). We examined the marker gene set in a phenylbutyrate dose-finding study that was recently performed in HD patients. Interestingly, we found significant downregulation of a subset of mRNAs in treated patients suggesting that HDAC inhibitors may affect specific targets in blood of HD patients. Although the effects of HDAC inhibitors on expression of the marker genes appeared rather subtle, they were statistically significant in ten out of twelve patients.

To further address the question of biological relevance of the marker genes, we asked whether these mRNAs were differentially expressed in human HD brain. Of the 12 genes tested, 7 showed significantly unregulated expression in postmortem HD brain suggesting that mutant huntingtin may affect similar targets in blood and brain. It has been previously demonstrated that mutant huntingtin specifically interferes with the function of general transcription factors and coactivators, such as Sp1 and CBP (Sugars, K. L. et al., Trends in Genetics 19: 233-238, 2003; Dunah, A. W. et al., Science 296: 2238-2243, 2002). Since these factors exhibit ubiquitous expression, mutant huntingtin may affect gene transcription in tissues outside the CNS. At present time, it is not clear why only neurons are predominantly affected in HD. One possible explanation may lie in the fact that neurons are post-mitotic and highly specialized, as opposed to dividing blood cells that may be less susceptible to degeneration because of their short half-life.

Microarrays for gene expression profiling are rapidly becoming important research tools for identifying potential biomarkers (Welsh, J. B. et al., Proc. Natl. Acad. Sci. USA 98: 1176-1181, 2001; Staal, F. J. et al., Leukemia 17: 1324-1332, 2003; Ilyin, S. E. et al., Trends Biotechnol. 22: 411-416, 2004). In contrast to other studies related to HD that have used tissues difficult to access in live patients, we used easily accessible peripheral blood. The use of genomic research in the analysis of blood-derived MRNA may represent an important advancement for development of biomarkers in neurological and other diseases. Such a minimally invasive approach enables larger sample sizes, better matching of patients and more standardized collection procedures. We have identified a large number of significantly altered mRNAs from which other smaller sets of genes could be selected and validated in a variety of clinical situations. The selection and validation of marker genes could vary depending on the purpose of the biomarker. For example, biomarkers used for prognostic purposes might differ from biomarkers linking a surrogate endpoint to a clinical endpoint and would be validated differently. Furthermore, the optimal biomarker gene set for monitoring different therapeutic targets could differ but could potentially be configured from the larger pool of genes we have identified. One of the goals of biomarker development relevant to HD and other diseases is to enable an assessment of drug specificity, safety and efficacy, thereby improving the efficiency and cost-effectiveness of the drug development process.

TABLE 1A

| HD | | |
|---|---|---|
| Bio- | | |
| marker | Affymetrix | |
| Number | probe set | Affymetrix Descriptions |
| 1 | 200037_s_at | gb: NM_016587.1 /DEF = Homo sapiens heterochromatin-like protein 1 (HECH), mRNA. /FEA = mRNA /GEN = HECH |
| | | /PROD = heterochromatin-like protein 1 /DB_XREF = gi: 7705406 /UG = Hs.278554 heterochromatin-like protein 1 |
| | | /FL = gb: AF136630.1 gb: NM_016587.1 |
| 2 | 200084_at | Consensus includes gb: BE748698 /FEA = EST /DB_XREF = gi: 10162690 /DB_XREF = est: 601571740T1 |
| | | /CLONE = IMAGE: 3838712 /UG = Hs.78050 small acidic protein |
| 3 | 200608_s_at | gb: NM_006265.1 /DEF = Homo sapiens RAD21 (S. pombe) homolog (RAD21), mRNA. /FEA = mRNA /GEN = RAD21 |
| | | /PROD = RAD21 (S. pombe) homolog /DB_XREF = gi: 5453993 /UG = Hs.81848 RAD21 (S. pombe) homolog |
| | | /FL = gb: D38551.1 gb: NM_006265.1 |
| 4 | 200626_s_at | gb: NM_018834.1 /DEF = Homo sapiens matrin 3 (MATR3), mRNA. /FEA = mRNA /GEN = MATR3 /PROD = matrin 3 |
| | | /DB_XREF = gi: 10047081 /UG = Hs.78825 matrin 3 /FL = gb: NM_018834.1 gb: AB018266.1 |
| 5 | 200668_s_at | gb: BC003395.1 /DEF = Homo sapiens, ubiquitin-conjugating = enzyme E2D 3 (homologous to yeast UBC45), clone |
| | | MGC: 5416, mRNA, complete cds. /FEA = mRNA /PROD = ubiquitin-conjugating = enzyme E2D 3 (homologousto yeast |
| | | UBC45) /DB_XREF = gi: 13097281 /UG = Hs.118797 ubiquiti |
| 6 | 200706_s_at | gb: NM_004862.1 /DEF = Homo sapiens LPS-induced TNF-alpha factor (PIG7), mRNA. /FEA = mRNA /GEN = PIG7 |
| | | /PROD = LPS-induced TNF-alpha factor /DB_XREF = gi: 4758913 /UG = Hs.76507 LPS-induced TNF-alpha factor |
| | | /FL = gb: AB034747.1 gb: U77396.1 gb: AF010312.1 gb: NM_004862.1 |
| 7 | 200726_at | gb: NM_002710.1 /DEF = Homo sapiens protein phosphatase 1, catalytic subunit, gamma isoform (PPP1CC), mRNA. |
| | | /FEA = mRNA /GEN = PPP1CC /PROD = protein phosphatase 1, catalytic subunit, gammaisoform /DB_XREF = gi: |
| | | 4506006 /UG = Hs.79081 protein phosphatase 1, catalytic |
| 8 | 200761_s_at | gb: NM_006407.2 /DEF = Homo sapiens vitamin A responsive; cytoskeleton related (JWA), mRNA. /FEA = mRNA |
| | | /GEN = JWA /PROD = vitamin A responsive; cytoskeleton related /DB_XREF = gi: 7669496 /UG = Hs.92384 vitamin A |
| | | responsive; cytoskeleton related /FL = gb: BC005143.1 gb: |
| 9 | 200777_s_at | gb: NM_014670.1 /DEF = Homo sapiens KIAA0005 gene product (KIAA0005), mRNA. /FEA = mRNA /GEN = KIAA0005 |
| | | /PROD = KIAA0005 gene product /DB_XREF = gi: 7661849 /UG = Hs.155291 KIAA0005 gene product /FL = gb: |
| | | D13630.1 gb: NM_014670.1 |
| 10 | 200821_at | gb: NM_013995.1 /DEF = Homo sapiens lysosomal-associated membrane protein 2 (LAMP2), transcript variant LAMP2B, |
| | | mRNA. /FEA = mRNA /GEN = LAMP2 /PROD = lysosomal-associated membrane protein 2precursor |
| | | /DB_XREF = gi: 7669502 /UG = Hs.8262 lysosomal-associated membrane p |
| 11 | 200833_s_at | gb: NM_015646.1 /DEF = Homo sapiens RAP1B, member of RAS oncogene family (RAP1B), mRNA. /FEA = mRNA |
| | | /GEN = RAP1B /PROD = DKFZP586H0723 protein /DB_XREF = gi: 7661677 /UG = Hs.156764 RAP1B, member of RAS |
| | | oncogene family /FL = gb: BC000176.2 gb: NM_015646.1 |
| 12 | 200847_s_at | gb: NM_016127.1 /DEF = Homo sapiens HSPC035 protein (LOC51669), mRNA. /FEA = mRNA /GEN = LOC51669 |
| | | /PROD = HSPC035 protein /DB_XREF = gi: 7706384 /UG = Hs.279921 HSPC035 protein /FL = gb: AF100748.1 |
| | | gb: AF078855.1 gb: NM_016127.1 |
| 13 | 200891_s_at | gb: NM_003144.2 /DEF = Homo sapiens signal sequence receptor, alpha (translocon-associated protein alpha) (SSR1), |
| | | mRNA. /FEA = mRNA /GEN = SSR1 /PROD = signal sequence receptor, alpha /DB_XREF = gi: 6552340 /UG = |
| | | Hs.250773 signal sequence receptor, alpha (translocon-a |
| 14 | 200898_s_at | Consensus includes gb: AK002091.1 /DEF = Homo sapiens cDNA FLJ11229 fis, clone PLACE1008356, highly similar to |
| | | Homo sapiens mRNA for KIAA0679 protein. /FEA = mRNA /DB_XREF = gi: 7023763 /UG = Hs.5734 meningioma |
| | | expressed antigen 5 (hyaluronidase) /FL = gb: AF036144.2 |
| 15 | 200902_at | gb: NM_004261.1 /DEF = Homo sapiens 15 kDa selenoprotein (SEP15), mRNA. /FEA = mRNA /GEN = SEP15 |
| | | /PROD = 15 kDa selenoprotein /DB_XREF = gi: 4759095 /UG = Hs.90606 15 kDa selenoprotein /FL = gb: AF288991.1 |
| | | gb: BC005294.1 gb: AF051894.1 gb: NM_004261.1 |
| 16 | 200912_s_at | gb: NM_001967.2 /DEF = Homo sapiens eukaryotic translation initiation factor 4A, isoform 2 (EIF4A2), mRNA. |
| | | /FEA = mRNA /GEN = EIF4A2 /PROD = eukaryotic translation initiation factor 4A, isoform 2 /DB_XREF = gi: 9945313 |
| | | /UG = Hs.173912 eukaryotic translation initiation |
| 17 | 200915_x_at | gb: NM_004986.1 /DEF = Homo sapiens kinectin 1 (kinesin receptor) (KTN1), mRNA. /FEA = mRNA /GEN = KTN1 |
| | | /PROD = kinectin 1 (kinesin receptor) /DB_XREF = gi: 4826813 /UG = Hs.211577 kinectin 1 (kinesin receptor) |
| | | /FL = gb: D13629.1 gb: L25616.1 gb: NM_004986.1 |
| 18 | 200934_at | gb: NM_003472.1 /DEF = Homo sapiens DEK oncogene (DNA binding) (DEK), mRNA. /FEA = mRNA /GEN = DEK |
| | | /PROD = DEK oncogene (DNA binding) /DB_XREF = gi: 4503248 /UG = Hs.110713 DEK oncogene (DNA binding) |
| | | /FL = gb: NM_003472.1 |
| 19 | 200970_s_at | gb: AL136807.1 /DEF = Homo sapiens mRNA; cDNA DKFZp434L1621 (from clone DKFZp434L1621); complete cds. |
| | | /FEA = mRNA /GEN = DKFZp434L1621 /PROD = hypothetical protein /DB_XREF = gi: 12053124 /UG = Hs.76698 stress- |
| | | associated endoplasmic reticulum protein 1; ribosome assoc |
| 20 | 200989_at | gb: NM_001530.1 /DEF = Homo sapiens hypoxia-inducible factor 1, alpha subunit (basic helix-loop-helix transcription |
| | | factor) (HIF1A), mRNA. /FEA = mRNA /GEN = HIF1A /PROD = hypoxia-inducible factor 1, alpha subunit (basichelix-loop- |
| | | helix transcription factor) /DB_{—} |
| 21 | 201012_at | gb: NM_000700.1 /DEF = Homo sapiens annexin A1 (ANXA1), mRNA. /FEA = mRNA /GEN = ANXA1 /PROD = |
| | | annexin I /DB_XREF = gi: 4502100 /UG = Hs.78225 annexin A1 /FL = gb: BC001275.1 gb: NM_000700.1 |
| 22 | 201023_at | gb: NM_005642.1 /DEF = Homo sapiens TATA box binding protein (TBP)-associated factor, RNA polymerase II, F, 55 kD |
| | | (TAF2F), mRNA. /FEA = mRNA /GEN = TAF2F /PROD = TATA box binding protein (TBP)-associatedfactor, RNA |
| | | polymerase II, F, 55 kD /DB_XREF = gi: 5032148 /UG = Hs |
| 23 | 201071_x_at | gb: NM_012433.1 /DEF = Homo sapiens splicing factor 3b, subunit 1, 155 kD (SF3B1), mRNA. /FEA = mRNA |
| | | /GEN = SF3B1 /PROD = splicing factor 3b, subunit 1, 155 kD /DB_XREF = gi: 6912653 /UG = Hs.13453 splicing |
| | | factor 3b, subunit 1, 155 kD /FL = gb: AF054284.1 gb: NM_012433.1 |
| 24 | 201084_s_at | gb: NM_014739.1 /DEF = Homo sapiens KIAA0164 gene product (KIAA0164), mRNA. /FEA = mRNA /GEN = KIAA0164 |
| | | /PROD = KIAA0164 gene product /DB_XREF = gi: 7661957 /UG = Hs.80338 KIAA0164 gene product /FL = gb: D79986.1 |
| | | gb: NM_014739.1 |
| 25 | 201129_at | gb: NM_006276.2 /DEF = Homo sapiens splicing factor, arginineserine-rich 7 (35 kD) (SFRS7), mRNA. /FEA = mRNA |
| | | /GEN = SFRS7 /PROD = splicing factor, arginineserine-rich 7 (35 kD) /DB_XREF = gi: 6857827 /UG = Hs.184167 splicing |
| | | factor, arginineserine-rich 7 (35 kD) /FL = gb: |
| 26 | 201133_s_at | Consensus includes gb: AA142966 /FEA = EST /DB_XREF = gi: 1712344 /DB_XREF = est: zl43b05.s1 |
| | | /CLONE = IMAGE: 504657 /UG = Hs.279849 KIAA0438 gene product /FL = gb: AB007898.1 gb: NM_014819.1 |
| 27 | 201146_at | gb: NM_006164.1 /DEF = Homo sapiens nuclear factor (erythroid-derived 2)-like 2 (NFE2L2), mRNA. /FEA = mRNA |
| | | /GEN = NFE2L2 /PROD = nuclear factor (erythroid-derived 2)-like 2 /DB_XREF = gi: 5453775 /UG = Hs.155396 nuclear |
| | | factor (erythroid-derived 2)-like 2 /FL = gb: NM_0 |
| 28 | 201177_s_at | gb: NM_005499.1 /DEF = Homo sapiens SUMO-1 activating enzyme subunit 2 (UBA2), mRNA. /FEA = mRNA |
| | | /GEN = UBA2 /PROD = SUMO-1 activating enzyme subunit 2 /DB_XREF = gi: 4885648 /UG = Hs.4311 SUMO-1 |
| | | activating enzyme subunit 2 /FL = gb: BC003153.1 gb: U35832.1 gb: AF090384.1 g |
| 29 | 201200_at | gb: NM_003851.1 /DEF = Homo sapiens cellular repressor of E1A-stimulated genes (CREG), mRNA. /FEA = mRNA |
| | | /GEN = CREG /PROD = cellular repressor of E1A-stimulated genes /DB_XREF = gi: 4503036 /UG = Hs.5710 cellular |
| | | repressor of E1A-stimulated genes /FL = gb: AF084523.1 gb |
| 30 | 201210_at | gb: NM_001356.2 /DEF = Homo sapiens DEADH (Asp-Glu-Ala-AspHis) box polypeptide 3 (DDX3), transcript variant 2, |
| | | mRNA. /FEA = mRNA /GEN = DDX3 /PROD = DEADH (Asp-Glu-Ala-AspHis) box polypeptide 3 /DB_XREF = |
| | | gi: 13514812 /UG = Hs.147916 DEADH (Asp-Glu-Ala-AspHis) box pol |
| 31 | 201237_at | Consensus includes gb: AV685920 /FEA = EST /DB_XREF = gi: 10287783 /DB_XREF = est: AV685920 /CLONE = |
| | | GKCEGD05 /UG = Hs.75546 capping protein (actin filament) muscle Z-line, alpha 2 /FL = gb: BC005338.1 gb: |
| | | NM_006136.1 gb: U03269.1 |
| 32 | 201238_s_at | gb: BC005338.1 /DEF = Homo sapiens, capping protein (actin filament) muscle Z-line, alpha 2, clone MGC: 12426, mRNA, |
| | | complete cds. /FEA = mRNA /PROD = capping protein (actin filament) muscle Z-line, alpha 2 /DB_XREF = gi: 13529130 |
| | | /UG = Hs.75546 capping protein (actin |
| 33 | 201257_x_at | gb: NM_001006.1 /DEF = Homo sapiens ribosomal protein S3A (RPS3A), mRNA. /FEA = mRNA /GEN = RPS3A |
| | | /PROD = ribosomal protein S3A /DB_XREF = gi: 4506722 /UG = Hs.77039 ribosomal protein S3A /FL = gb: BC000204.1 |
| | | gb: BC001708.1 gb: BC004981.1 gb: M84711.1 gb: M77234.1 gb: L13802.1 |
| 34 | 201297_s_at | Consensus includes gb: AK023321.1 /DEF = Homo sapiens cDNA FLJ13259 fis, clone OVARC1000876, moderately |
| | | similar to MOB1 PROTEIN. /FEA = mRNA /DB_XREF = gi: 10435206 /UG = Hs.196437 hypothetical protein FLJ10788 |
| | | /FL = gb: AB016839.1 gb: BC003398.1 gb: NM_018221.1 |
| 35 | 201312_s_at | gb: NM_003022.1 /DEF = Homo sapiens SH3 domain binding glutamic acid-rich protein like (SH3BGRL), mRNA. |
| | | /FEA = mRNA /GEN = SH3BGRL /PROD = SH3 domain binding glutamic acid-rich proteinlike /DB_XREF = gi: 4506924 |
| | | /UG = Hs.14368 SH3 domain binding glutamic acid-rich pro |
| 36 | 201409_s_at | gb: NM_002709.1 /DEF = Homo sapiens protein phosphatase 1, catalytic subunit, beta isoform (PPP1CB), mRNA. |
| | | /FEA = mRNA /GEN = PPP1CB /PROD = protein phosphatase 1, catalytic subunit, betaisoform /DB_XREF = gi: 4506004 |
| | | /UG = Hs.21537 protein phosphatase 1, catalytic su |
| 37 | 201450_s_at | gb: NM_022037.1 /DEF = Homo sapiens TIA1 cytotoxic granule-associated RNA-binding protein (TIA1), transcript variant |
| | | 1, mRNA. /FEA = mRNA /GEN = TIA1 /PROD = TIA1 protein, isoform 1 /DB_XREF = gi: 11863160 /UG = |
| | | Hs.239489 TIA1 cytotoxic granule-associated RNA-binding |
| 38 | 201458_s_at | gb: NM_004725.1 /DEF = Homo sapiens BUB3 (budding uninhibited by benzimidazoles 3, yeast) homolog (BUB3), mRNA. |
| | | /FEA = mRNA /GEN = BUB3 /PROD = BUB3 (budding uninhibited by benzimidazoles 3, yeast) homolog |
| | | /DB_XREF = gi: 4757879 /UG = Hs.40323 BUB3 (budding uninhibited |
| 39 | 201472_at | gb: NM_003372.2 /DEF = Homo sapiens von Hippel-Lindau binding protein 1 (VBP1), mRNA. /FEA = mRNA |
| | | /GEN = VBP1 /PROD = von Hippel-Lindau binding protein 1 /DB_XREF = gi: 9257253 /UG = Hs.198307 von |
| | | Hippel-Lindau binding protein 1 /FL = gb: U96759.1 gb: NM_003372.2 |
| 40 | 201487_at | gb: NM_001814.1 /DEF = Homo sapiens cathepsin C (CTSC), mRNA. /FEA = mRNA /GEN = CTSC /PROD = cathepsin C |
| | | /DB_XREF = gi: 4503140 /UG = Hs.10029 cathepsin C /FL = gb: NM_001814.1 |
| 41 | 201493_s_at | Consensus includes gb: BE778078 /FEA = EST /DB_XREF = gi: 10199276 /DB_XREF = est: 601463189F1 |
| | | /CLONE = IMAGE: 3866399 /UG = Hs.6151 pumilio (Drosophila) homolog 2 /FL = gb: AF315591.1 gb: NM_015317.1 |
| 42 | 201523_x_at | Consensus includes gb: BE262760 /FEA = EST /DB_XREF = gi: 9136144 /DB_XREF = est: 601153762F1 |
| | | /CLONE = IMAGE: 3509895 /UG = Hs.75355 ubiquitin-conjugating enzyme E2N (homologous to yeast UBC13) |
| | | /FL = gb: D83004.1 gb: BC000396.1 gb: BC003365.1 gb: NM_003348.1 |
| 43 | 201535_at | gb: NM_007106.1 /DEF = Homo sapiens ubiquitin-like 3 (UBL3), mRNA. /FEA = mRNA /GEN = UBL3 /PROD = |
| | | ubiquitin-like 3 /DB_XREF = gi: 6005927 /UG = Hs.173091 ubiquitin-like 3 /FL = gb: AF044221.1 gb: AL080177.1 |
| | | gb: NM_007106.1 |
| 44 | 201568_at | gb: NM_014402.1 /DEF = Homo sapiens low molecular mass ubiquinone-binding protein (9.5 kD) (QP-C), mRNA. |
| | | /FEA = mRNA /GEN = QP-C /PROD = low molecular mass ubiquinone-binding protein /DB_XREF = gi: 7657485 |
| | | /UG = Hs.3709 low molecular mass ubiquinone-binding protein (9. |
| 45 | 201595_s_at | gb: NM_018471.1 /DEF = Homo sapiens uncharacterized hypothalamus protein HT010 (HT010), mRNA. /FEA = mRNA |
| | | /GEN = HT010 /PROD = uncharacterized hypothalamus protein HT010 /DB_XREF = gi: 8923807 /UG = Hs.6375 |
| | | uncharacterized hypothalamus protein HT010 /FL = gb: AF220184.1 |
| 46 | 201604_s_at | gb: NM_002480.1 /DEF = Homo sapiens myosin phosphatase, target subunit 1 (MYPT1), mRNA. /FEA = mRNA |
| | | /GEN = MYPT1 /PROD = myosin phosphatase target subunit 1 /DB_XREF = gi: 4505316 /UG = Hs.16533 myosin |
| | | phosphatase, target subunit 1 /FL = gb: NM_002480.1 |
| 47 | 201699_at | gb: NM_002806.1 /DEF = Homo sapiens proteasome (prosome, macropain) 26S subunit, ATPase, 6 (PSMC6), mRNA. |
| | | /FEA = mRNA /GEN = PSMC6 /PROD = proteasome (prosome, macropain) 26S subunit, ATPase, 6 /DB_XREF = |
| | | gi: 4506214 /UG = Hs.79357 proteasome (prosome, macropain) 26S s |
| 48 | 201761_at | gb: NM_006636.2 /DEF = Homo sapiens methylene tetrahydrofolate dehydrogenase (NAD+ dependent), |
| | | methenyltetrahydrofolate cyclohydrolase (MTHFD2), nuclear gene encoding mitochondrial protein, mRNA. /FEA = mRNA |
| | | /GEN = MTHFD2 /PROD = methylene tetrahydrofolate dehydr |
| 49 | 201780_s_at | gb: NM_007282.1 /DEF = Homo sapiens ring finger protein 13 (RNF13), mRNA. /FEA = mRNA /GEN = RNF13 |
| | | /PROD = ring finger protein 13 /DB_XREF = gi: 6005863 /UG = Hs.6900 ring finger protein 13 /FL = gb: AF037204.1 |
| | | gb: AF070558.1 gb: NM_007282.1 |
| 50 | 201807_at | gb: NM_004896.1 /DEF = Homo sapiens vacuolar protein sorting 26 (yeast homolog) (VPS26), mRNA. /FEA = mRNA |
| | | /GEN = VPS26 /PROD = vacuolar protein sorting 26 (yeast homolog) /DB_XREF = gi: 4758509 /UG = Hs.67052 vacuolar |
| | | protein sorting 26 (yeast homolog) /FL = gb: AF05417 |
| 51 | 201857_at | gb: NM_016107.1 /DEF = Homo sapiens M-phase phosphoprotein homolog (LOC51663), mRNA. /FEA = mRNA |
| | | /GEN = LOC51663 /PROD = M-phase phosphoprotein homolog /DB_XREF = gi: 7706372 /UG = Hs.173518 M-phase |
| | | phosphoprotein homolog /FL = gb: BC000376.1 gb: BC000746.1 gb: AF100742.1 g |
| 52 | 201859_at | gb: NM_002727.1 /DEF = Homo sapiens proteoglycan 1, secretory granule (PRG1), mRNA. /FEA = Mrna /GEN = PRG1 |
| | | /PROD = proteoglycan 1, secretory granule /DB_XREF = gi: 4506044 /UG = Hs.1908 proteoglycan 1, secretory granule |
| | | /FL = gb: J03223.1 gb: NM_002727.1 |
| 53 | 201952_at | Consensus includes gb: AA156721 /FEA = EST /DB_XREF = gi: 1728335 /DB_XREF = est: zl18b04.s1 |
| | | /CLONE = IMAGE: 502255 /UG = Hs.10247 activated leucocyte cell adhesion molecule /FL = gb: NM_001627.1 |
| | | gb: L38608.1 |
| 54 | 202006_at | gb: NM_002835.1 /DEF = Homo sapiens protein tyrosine phosphatase, non-receptor type 12 (PTPN12), mRNA. |
| | | /FEA = mRNA /GEN = PTPN12 /PROD = protein tyrosine phosphatase, non-receptor type12 /DB_XREF = gi: 4506286 |
| | | /UG = Hs.62 protein tyrosine phosphatase, non-receptor typ |
| 55 | 202020_s_at | gb: NM_006055.1 /DEF = Homo sapiens LanC (bacterial lantibiotic synthetase component C)-like 1 (LANCL1), mRNA. |
| | | /FEA = mRNA /GEN = LANCL1 /PROD = lanthionine synthetase C-like protein 1 /DB_XREF = gi: 5174444 |
| | | /UG = Hs.13351 LanC (bacterial lantibiotic synthetase compon |
| 56 | 202033_s_at | Consensus includes gb: BG402105 /FEA = EST /DB_XREF = gi: 13295553 /DB_XREF = est: 602465641F1 |
| | | /CLONE = IMAGE: 4593682 /UG = Hs.50421 KIAA0203 gene product /FL = gb: D86958.1 gb: NM_014781.1 |
| 57 | 202076_at | gb: NM_001166.2 /DEF = Homo sapiens baculoviral IAP repeat-containing 2 (BIRC2), mRNA. /FEA = mRNA |
| | | /GEN = BIRC2 /PROD = baculoviral IAP repeat-containing protein 2 /DB_XREF = gi: 10880127 /UG = Hs.289107 |
| | | baculoviral IAP repeat-containing 2 /FL = gb: NM_001166.2 gb: U37547. |
| 58 | 202113_s_at | gb: AF043453.1 /DEF = Homo sapiens sorting nexin 2 (SNX2) mRNA, complete cds. /FEA = mRNA /GEN = SNX2 |
| | | /PROD = sorting nexin 2 /DB_XREF = gi: 2827433 /UG = Hs.11183 sorting nexin 2 /FL = gb: BC003382.1 gb: AF043453.1 |
| | | gb: AF065482.1 gb: NM_003100.1 |
| 59 | 202141_s_at | gb: BC003090.1 /DEF = Homo sapiens, COP9 homolog, clone MGC: 1297, mRNA, complete cds. /FEA = mRNA |
| | | /PROD = COP9 homolog /DB_XREF = gi: 13111846 /UG = Hs.75193 COP9 homolog /FL = gb: BC003090.1 gb: U51205.1 |
| | | gb: NM_006710.1 |
| 60 | 202147_s_at | gb: NM_001550.1 /DEF = Homo sapiens interferon-related developmental regulator 1 (IFRD1), mRNA. /FEA = mRNA |
| | | /GEN = IFRD1 /PROD = interferon-related developmental regulator 1 /DB_XREF = gi: 4504606 /UG = Hs.7879 interferon- |
| | | related developmental regulator 1 /FL = gb: BC001 |
| 61 | 202164_s_at | gb: AF180476.1 /DEF = Homo sapiens CALIFp (CALIF) mRNA, complete cds. /FEA = mRNA /GEN = CALIF |
| | | /PROD = CALIFp /DB_XREF = gi: 6856208 /UG = Hs.26703 CCR4-NOT transcription complex, subunit 8 |
| | | /FL = gb: AF053318.1 gb: NM_004779.1 gb: AL122045.1 gb: AF180476.1 |
| 62 | 202166_s_at | gb: NM_006241.1 /DEF = Homo sapiens protein phosphatase 1, regulatory (inhibitor) subunit 2 (PPP1R2), mRNA. |
| | | /FEA = mRNA /GEN = PPP1R2 /PROD = protein phosphatase 1, regulatory (inhibitor)subunit 2 /DB_XREF = gi: 5453945 |
| | | /UG = Hs.267819 protein phosphatase 1, regulator |
| 63 | 202168_at | gb: NM_003187.1 /DEF = Homo sapiens TATA box binding protein (TBP)-associated factor, RNA polymerase II, G, 32 kD |
| | | (TAF2G), mRNA. /FEA = mRNA /GEN = TAF2G /PROD = TATA box binding protein (TBP)-associatedfactor, RNA |
| | | polymerase II, G, 32 kD /DB_XREF = gi: 4507350 /UG = Hs |
| 64 | 202169_s_at | gb: AF302110.1 /DEF = Homo sapiens alpha-aminoadipic semialdehyde dehydrogenase-phosphopantetheinyl transferase |
| | | mRNA, complete cds. /FEA = mRNA /PROD = alpha-aminoadipic semialdehydedehydrogenase-phosphopantetheinyl |
| | | transferase /DB_XREF = gi: 11120434 /UG = Hs.64595 |
| 65 | 202175_at | gb: NM_024536.1 /DEF = Homo sapiens hypothetical protein FLJ22678 (FLJ22678), mRNA. /FEA = mRNA |
| | | /GEN = FLJ22678 /PROD = hypothetical protein FLJ22678 /DB_XREF = gi: 13375692 /UG = Hs.7718 hypothetical protein |
| | | FLJ22678 /FL = gb: NM_024536.1 |
| 66 | 202194_at | Consensus includes gb: AL117354 /DEF = Human DNA sequence from clone RP5-976O13 on chromosome 1p21.2-22.2 |
| | | Contains part of the gene for CGI-100 protein, 3 isoforms of the gene for M96 protein, ESTs, STSs, GSSs and a CpG |
| | | Island /FEA = mRNA_1 /DB_XREF = gi: 6822199 |
| 67 | 202214_s_at | gb: NM_003588.1 /DEF = Homo sapiens cullin 4B (CUL4B), mRNA. /FEA = mRNA /GEN = CUL4B /PROD = cullin 4B |
| | | /DB_XREF = gi: 13270466 /UG = Hs.155976 cullin 4B /FL = gb: NM_003588.1 gb: AB014595.1 |
| 68 | 202228_s_at | gb: NM_017455.1 /DEF = Homo sapiens stromal cell derived factor receptor 1 (SDFR1), transcript variant alpha, mRNA. |
| | | /FEA = mRNA /GEN = SDFR1 /PROD = stromal cell derived factor receptor 1 isoforma /DB_XREF = gi: 9257239 |
| | | /UG = Hs.6354 stromal cell derived factor recept |
| 69 | 202258_s_at | Consensus includes gb: U50532.1 /DEF = Human BRCA2 region, mRNA sequence CG005. /FEA = mRNA |
| | | /PROD = unknown /DB_XREF = gi: 1531603 /UG = Hs.23518 hypothetical protein from BCRA2 region /FL = gb: |
| | | NM_014887.1 |
| 70 | 202266_at | gb: NM_016614.1 /DEF = Homo sapiens TRAF and TNF receptor-associated protein (AD022), mRNA. /FEA = mRNA |
| | | /GEN = AD022 /PROD = TRAF and TNF receptor-associated protein /DB_XREF = gi: 7705261 /UG = Hs.46847 TRAF and |
| | | TNF receptor-associated protein /FL = gb: AF201687.1 gb: AF |
| 71 | 202318_s_at | gb: AF306508.1 /DEF = Homo sapiens SUMO-1 specific protease FKSG6 mRNA, complete cds. /FEA = mRNA |
| | | /PROD = SUMO-1 specific protease FKSG6 /DB_XREF = gi: 11096243 /UG = Hs.27197 SUMO-1-specific protease |
| | | /FL = gb: AF307849.1 gb: AF306508.1 gb: AF196304.1 gb: NM_015571.1 |
| 72 | 202353_s_at | gb: NM_002816.1 /DEF = Homo sapiens proteasome (prosome, macropain) 26S subunit, non-ATPase, 12 (PSMD12), |
| | | mRNA. /FEA = mRNA /GEN = PSMD12 /PROD = proteasome (prosome, macropain) 26S subunit, non-ATPase, 12 |
| | | /DB_XREF = gi: 4506220 /UG = Hs.4295 proteasome (prosome, macro |
| 73 | 202370_s_at | gb: NM_001755.1 /DEF = Homo sapiens core-binding factor, beta subunit (CBFB), transcript variant 2, mRNA. |
| | | /FEA = mRNA /GEN = CBFB /PROD = core-binding factor, beta subunit, isoform 2 /DB_XREF = gi: 13124872 |
| | | /UG = Hs.179881 core-binding factor, beta subunit /FL = gb: NM_0 |
| 74 | 202381_at | gb: NM_003816.1 /DEF = Homo sapiens a disintegrin and metalloproteinase domain 9 (meltrin gamma) (ADAM9), mRNA. |
| | | /FEA = mRNA /GEN = ADAM9 /PROD = a disintegrin and metalloproteinase domain 9preproprotein /DB_XREF = |
| | | gi: 4501914 /UG = Hs.2442 a disintegrin and metallopro |
| 75 | 202422_s_at | gb: NM_022977.1 /DEF = Homo sapiens fatty-acid-Coenzyme A ligase, long-chain 4 (FACL4), transcript variant 2, mRNA. |
| | | /FEA = mRNA /GEN = FACL4 /PROD = long-chain fatty-acid-Coenzyme A ligase 4, isoform 2 /DB_XREF = gi: 12669908 |
| | | /UG = Hs.81452 fatty-acid-Coenzyme A ligase |
| 76 | 202429_s_at | gb: AL353950.1 /DEF = Homo sapiens mRNA; cDNA DKFZp761L0516 (from clone DKFZp761L0516); complete cds. |
| | | /FEA = mRNA /GEN = DKFZp761L0516 /PROD = hypothetical protein /DB_XREF = gi: 7669991 /UG = |
| | | Hs.272458 protein phosphatase 3 (formerly 2B), catalytic subunit, alpha iso |
| 77 | 202432_at | gb: NM_021132.1 /DEF = Homo sapiens protein phosphatase 3 (formerly 2B), catalytic subunit, beta isoform (calcineurin A |
| | | beta) (PPP3CB), mRNA. /FEA = mRNA /GEN = PPP3CB /PROD = protein phosphatase 3 (formerly 2B), catalyticsubunit, |
| | | beta isoform (calcineurin A beta |
| 78 | 202437_s_at | gb: NM_000104.2 /DEF = Homo sapiens cytochrome P450, subfamily I (dioxin-inducible), polypeptide 1 (glaucoma 3, |
| | | primary infantile) (CYP1B1), mRNA. /FEA = mRNA /GEN = CYP1B1 /PROD = cytochrome P450, subfamily I (dioxin- |
| | | inducible), polypeptide 1 /DB_XREF = gi: 13325059 |
| 79 | 202467_s_at | gb: NM_004236.1 /DEF = Homo sapiens thyroid receptor interacting protein 15 (TRIP15), mRNA. /FEA = mRNA |
| | | /GEN = TRIP15 /PROD = thyroid receptor interacting protein 15 /DB_XREF = gi: 4759263 /UG = Hs.30212 |
| | | thyroid receptor interacting protein 15 /FL = gb: AF084260.1 gb: NM_{—} |
| 80 | 202506_at | gb: NM_006751.1 /DEF = Homo sapiens sperm specific antigen 2 (SSFA2), mRNA. /FEA = mRNA /GEN = SSFA2 |
| | | /PROD = sperm specific antigen 2 /DB_XREF = gi: 5803178 /UG = Hs.82767 sperm specific antigen 2 /FL = gb: M61199.1 |
| | | gb: NM_006751.1 |
| 81 | 202536_at | Consensus includes gb: AK002165.1 /DEF = Homo sapiens cDNA FLJ11303 fis, clone PLACE1009995, highly similar to |
| | | Homo sapiens mRNA; cDNA DKFZp564O123. /FEA = mRNA /DB_XREF = gi: 7023876 /UG = Hs.11449 |
| | | DKFZP564O123 protein /FL = gb: AF151842.1 gb: AL080122.1 gb: NM_014043 |
| 82 | 202544_at | gb: NM_004124.1 /DEF = Homo sapiens glia maturation factor, beta (GMFB), mRNA. /FEA = mRNA /GEN = GMFB |
| | | /PROD = glia maturation factor, beta /DB_XREF = gi: 4758441 /UG = Hs.151413 glia maturation factor, beta |
| | | /FL = gb: BC005359.1 gb: M86492.1 gb: AB001106.1 gb: NM_004124.1 |
| 83 | 202594_at | gb: NM_015344.1 /DEF = Homo sapiens MY047 protein (MY047), mRNA. /FEA = mRNA /GEN = MY047 |
| | | /PROD = MY047 protein /DB_XREF = gi: 7662509 /UG = Hs.11000 leptin receptor overlapping transcript-like 1 |
| | | /FL = gb: BC000642.1 gb: AF063605.1 gb: AF161461.1 gb: NM_015344.1 |
| 84 | 202606_s_at | gb: NM_012290.1 /DEF = Homo sapiens tousled-like kinase 1 (TLK1), mRNA. /FEA = mRNA /GEN = TLK1 |
| | | /PROD = tousled-like kinase 1 /DB_XREF = gi: 6912719 /UG = Hs.18895 tousled-like kinase 1 /FL_gb: AB004885.1 gb: |
| | | NM_012290.1 gb: AF246219.1 |
| 85 | 202651_at | gb: NM_014873.1 /DEF = Homo sapiens KIAA0205 gene product (KIAA0205), mRNA. /FEA = mRNA /GEN = KIAA0205 |
| | | /PROD = KIAA0205 gene product /DB_XREF = gi: 7661995 /UG = Hs.3610 KIAA0205 gene product /FL = gb: D86960.1 |
| | | gb: NM_014873.1 |
| 86 | 202653_s_at | Consensus includes gb: BC003404.1 /DEF = Homo sapiens, hypothetical protein DKFZp586F1122 similar to axotrophin, |
| | | clone IMAGE: 3449089, mRNA, partial cds. /FEA = mRNA /PROD = hypothetical protein DKFZp586F1122 similar |
| | | toaxotrophin /DB_XREF = gi: 13097302 /UG = Hs.5306 |
| 87 | 202654_x_at | gb: NM_022826.1 /DEF = Homo sapiens hypothetical protein DKFZp586F1122 similar to axotrophin (DKFZP586F1122), |
| | | mRNA. /FEA = mRNA /GEN = DKFZP586F1122 /PROD = hypothetical protein DKFZp586F1122 similar toaxotrophin |
| | | /DB_XREF = gi: 12383065 /UG = Hs.5306 hypothetical prot |
| 88 | 202673_at | gb: NM_003859.1 /DEF = Homo sapiens dolichyl-phosphate mannosyltransferase polypeptide 1, catalytic subunit (DPM1), |
| | | mRNA. /FEA = mRNA /GEN = DPM1 /PROD = dolichyl-phosphate mannosyltransferasepolypeptide 1 |
| | | /DB_XREF = gi: 4503362 /UG = Hs.5085 dolichyl-phosphate mannos |
| 89 | 202687_s_at | gb: U57059.1 /DEF = Homo sapiens Apo-2 ligand mRNA, complete cds. /FEA = mRNA /PROD = Apo-2 ligand |
| | | /DB_XREF = gi: 1336207 /UG = Hs.83429 tumor necrosis factor (ligand) superfamily, member 10 /FL = gb: U37518.1 |
| | | gb: U57059.1 gb: NM_003810.1 |
| 90 | 202763_at | gb: NM_004346.1 /DEF = Homo sapiens caspase 3, apoptosis-related cysteine protease (CASP3), mRNA. /FEA = mRNA |
| | | /GEN = CASP3 /PROD = caspase 3, apoptosis-related cysteine protease /DB_XREF = gi: 4757911 /UG = Hs.74552 caspase |
| | | 3, apoptosis-related cysteine protease /FL = g |
| 91 | 202769_at | Consensus includes gb: AW134535 /FEA = EST /DB_XREF = gi: 6138088 /DB_XREF = est: UI-H-BI1-abv-g-06-0-UI.s1 |
| | | /CLONE = IMAGE: 2713163 /UG = Hs.79069 cyclin G2 /FL = gb: U47414.1 gb: NM_004354.1 |
| 92 | 202777_at | gb: NM_007373.1 /DEF = Homo sapiens suppressor of clear, C. elegans, homolog of (SHOC2), mRNA. /FEA = mRNA |
| | | /GEN = SHOC2 /PROD = suppressor of clear, C. elegans, homolog of /DB_XREF = gi: 6677944 /UG = Hs.104315 soc-2 |
| | | (suppressor of clear, C. elegans) homolog /FL = gb: AF0 |
| 93 | 202778_s_at | gb: NM_003453.1 /DEF = Homo sapiens zinc finger protein 198 (ZNF198), mRNA. /FEA = mRNA /GEN = ZNF198 |
| | | /PROD = zinc finger protein 198 /DB_XREF = gi: 4508010 /UG = Hs.109526 zinc finger protein 198 /FL = gb: AF035374.1 |
| | | gb: AF060181.1 gb: NM_003453.1 |
| 94 | 202797_at | gb: NM_014016.1 /DEF = Homo sapiens KIAA0851 protein (KIAA0851), mRNA. /FEA = mRNA /GEN = KIAA0851 |
| | | /PROD = KIAA0851 protein /DB_XREF = gi: 7662337 /UG = Hs.5867 KIAA0851 protein /FL = gb: AB020658.1 |
| | | gb: AL136831.1 gb: NM_014016.1 |
| 95 | 202798_at | gb: NM_006323.1 /DEF = Homo sapiens SEC24 (S. cerevisiae) related gene family, member B (SEC24B), mRNA. |
| | | /FEA = mRNA /GEN = SEC24B /PROD = SEC24 (S. cerevisiae) related gene family, member B /DB_XREF = gi: 5454045 |
| | | /UG = Hs.7239 SEC24 (S. cerevisiae) related gene family, |
| 96 | 202829_s_at | gb: NM_005638.1 /DEF = Homo sapiens synaptobrevin-like 1 (SYBL1), mRNA. /FEA = mRNA /GEN = SYBL1 |
| | | /PROD = synaptobrevin-like 1 /DB_XREF = gi: 5032136 /UG = Hs.24167 synaptobrevin-like 1 /FL = gb: NM_005638.1 |
| 97 | 202902_s_at | gb: NM_904079.1 /DEF = Homo sapiens cathepsin S (CTSS), mRNA. /FEA = mRNA /GEN = CTSS /PROD = cathepsin S |
| | | /DB_XREF = gi: 4758097 /UG = Hs.181301 cathepsin S /FL = gb: BC002642.1 gb: M86553.1 gb: NM_004079.1 |
| | | gb: M90696.1 |
| 98 | 202906_s_at | Consensus includes gb: AF049895 /DEF = Homo sapiens 8q21.3: Nibrin (NBS1), 2,4-dienoyl-CoA reductase (DECR), and |
| | | calbindin 1 (CALB1) genes /FEA = mRNA_5 /DB_XREF = gi: 4126312 /UG = Hs.25812 Nijmegen breakage syndrome 1 |
| | | (nibrin) /FL = gb: AF058696.1 gb: AF051334.1 gb: N |
| 99 | 202918_s_at | gb: AF151853.1 /DEF = Homo sapiens CGI-95 protein mRNA, complete cds. /FEA = mRNA /PROD = CGI-95 protein |
| | | /DB_XREF = gi: 4929658 /UG = Hs.107942 DKFZP564M112 protein /FL = gb: AB015441.1 gb: BC005237.1 |
| | | gb: AF151853.1 gb: AL080070.1 gb: NM_015387.1 |
| 100 | 203008_x_at | gb: NM_005783.1 /DEF = Homo sapiens ATP binding protein associated with cell differentiation (APACD), mRNA. |
| | | /FEA = mRNA /GEN = APACD /PROD = ATP binding protein associated with celldifferentiation /DB_XREF = gi: 5031582 |
| | | /UG = Hs.153884 ATP binding protein associated w |
| 101 | 203020_at | gb: NM_014857.1 /DEF = Homo sapiens KIAA0471 gene product (KIAA0471), mRNA. /FEA = mRNA /GEN = KIAA0471 |
| | | /PROD = KIAA0471 gene product /DB_XREF = gi: 7662143 /UG = Hs.242271 KIAA0471 gene product |
| | | /FL = gb: AB007940.1 gb: NM_014857.1 |
| 102 | 203024_s_at | gb: NM_020199.1 /DEF = Homo sapiens HTGN29 protein (HTGN29), mRNA. /FEA = mRNA /GEN = HTGN29 |
| | | /PROD = HTGN29 protein /DB_XREF = gi: 9910277 /UG = Hs.283437 HTGN29 protein /FL = gb: AF226055.1 |
| | | gb: NM_020199.1 |
| 103 | 203049_s_at | gb: NM_014639.1 /DEF = Homo sapiens KIAA0372 gene product (KIAA0372), mRNA. /FEA = mRNA /GEN = KIAA0372 |
| | | /PROD = KIAA0372 gene product /DB_XREF = gi: 7662077 /UG = Hs.170098 KIAA0372 gene product |
| | | /FL = gb: AB002370.1 gb: NM_014639.1 |
| 104 | 203080_s_at | gb: NM_013450.1 /DEF = Homo sapiens bromodomain adjacent to zinc finger domain, 2B (BAZ2B), mRNA. |
| | | /FEA = mRNA /GEN = BAZ2B /PROD = bromodomain adjacent to zinc finger domain, 2B /DB_XREF = gi: 7304922 |
| | | /UG = Hs.8383 bromodomain adjacent to zinc finger domain, 2B /FL = gb |
| 105 | 203132_at | gb: NM_000321.1 /DEF = Homo sapiens retinoblastoma 1 (including osteosarcoma) (RB1), mRNA. /FEA = mRNA |
| | | /GEN = RB1 /PROD = retinoblastoma 1 (including osteosarcoma) /DB_XREF = gi: 4506434 /UG = Hs.75770 |
| | | retinoblastoma 1 (including osteosarcoma) /FL = gb: M33647.1 gb: M15400 |
| 106 | 203156_at | gb: NM_016248.1 /DEF = Homo sapiens A-kinase anchoring protein 220 (LOC51707), mRNA. /FEA = mRNA |
| | | /GEN = LOC51707 /PROD = A-kinase anchoring protein 220 /DB_XREF = gi: 7706456 /UG = Hs.232076 |
| | | A kinase (PRKA) anchor protein 11 /FL = gb: AF176555.1 gb: NM_016248.1 |
| 107 | 203224_at | Consensus includes gb: BF340123 /FEA = EST /DB_XREF = gi: 11286585 /DB_XREF = est: 602037283F1 |
| | | /CLONE = IMAGE: 4185212 /UG = Hs.37558 hypothetical protein FLJ11149 /FL = gb: NM_018339.1 |
| 108 | 203253_s_at | gb: NM_015216.1 /DEF = Homo sapiens KIAA0433 protein (KIAA0433), mRNA. /FEA = mRNA /GEN = KIAA0433 |
| | | /PROD = KIAA0433 protein /DB_XREF = gi: 7662117 /UG = Hs.26179 KIAA0433 protein /FL = gb: NM_015216.1 |
| 109 | 203299_s_at | gb: AF251295.1 /DEF = Homo sapiens DC22 mRNA, complete cds. /FEA = mRNA /PROD = DC22 /DB_XREF = |
| | | gi: 12005731 /UG = Hs.40368 adaptor-related protein complex 1, sigma 2 subunit /FL = gb: AF251295.1 gb: BC001117.1 |
| | | gb: AB015320.1 gb: NM_003916.1 |
| 110 | 203300_x_at | gb: NM_003916.1 /DEF = Homo sapiens adaptor-related protein complex 1, sigma 2 subunit (AP1S2), mRNA. |
| | | /FEA = mRNA /GEN = AP1S2 /PROD = adaptor-related protein complex 1, sigma 2 subunit /DB_XREF = gi: 4506956 |
| | | /UG = Hs.40368 adaptor-related protein complex 1, sigma 2 su |
| 111 | 203302_at | gb: NM_000788.1 /DEF = Homo sapiens deoxycytidine kinase (DCK), mRNA. /FEA = mRNA /GEN = DCK |
| | | /PROD = deoxycytidine kinase /DB_XREF = gi: 4503268 /UG = Hs.709 deoxycytidine kinase /FL = gb: M60527.1 |
| | | gb: NM_000788.1 |
| 112 | 203306_s_at | gb: NM_006416.1 /DEF = Homo sapiens solute carrier family 35 (CMP-sialic acid transporter), member 1 (SLC35A1), |
| | | mRNA. /FEA = mRNA /GEN = SLC35A1 /PROD = solute carrier family 35 (CMP-sialic acidtransporter), member 1 |
| | | /DB_XREF = gi: 5453620 /UG = Hs.82921 solute carrie |
| 113 | 203386_at | Consensus includes gb: AI650848 /FEA = EST /DB_XREF = gi: 4734827 /DB_XREF = est: wa95d04.x1 |
| | | /CLONE = IMAGE: 2303911 /UG = Hs.173802 KIAA0603 gene product /FL = gb: AB011175.1 gb: NM_014832.1 |
| 114 | 203387_s_at | gb: NM_014832.1 /DEF = Homo sapiens KIAA0603 gene product (KIAA0603), mRNA. /FEA = mRNA /GEN = KIAA0603 |
| | | /PROD = KIAA0603 gene product /DB_XREF = gi: 7662197 /UG = Hs.173802 KIAA0603 gene product /FL = |
| | | gb: AB011175.1 gb: NM_014832.1 |
| 115 | 203396_at | gb: NM_002789.1 /DEF = Homo sapiens proteasome (prosome, macropain) subunit, alpha type, 4 (PSMA4), mRNA. |
| | | /FEA = mRNA /GEN = PSMA4 /PROD = proteasome (prosome, macropain) subunit, alphatype, 4 /DB_XREF = gi: 4506184 |
| | | /UG = Hs.251531 proteasome (prosome, macropain) subu |
| 116 | 203397_s_at | Consensus includes gb: BF063271 /FEA = EST /DB_XREF = gi: 10822181 /DB_XREF = est: 7h87d05.x1 |
| | | /CLONE = IMAGE: 3322953 /UG = Hs.278611 UDP-N-acetyl-alpha-D-galactosamine: polypeptide N- |
| | | acetylgalactosaminyltransferase 3 (GalNAc-T3) /FL = gb: NM_004482.2 |
| 117 | 203403_s_at | gb: NM_005977.1 /DEF = Homo sapiens ring finger protein (C3H2C3 type) 6 (RNF6), mRNA. /FEA = mRNA |
| | | /GEN = RNF6 /PROD = ring finger protein (C3H2C3 type) 6 /DB_XREF = gi: 5174652 /UG = Hs.32597 ring finger protein |
| | | (C3H2C3 type) 6 /FL = gb: NM_005977.1 |
| 118 | 203420_at | gb: NM_016255.1 /DEF = Homo sapiens Autosomal Highly Conserved Protein (AHCP), mRNA. /FEA = mRNA |
| | | /GEN = AHCP /PROD = Autosomal Highly Conserved Protein /DB_XREF = gi: 7705267 /UG = Hs.95260 Autosomal |
| | | Highly Conserved Protein /FL = gb: AF097027.1 gb: NM_016255.1 |
| 119 | 203427_at | gb: NM_014034.1 /DEF = Homo sapiens DKFZP547E2110 protein (DKFZP547E2110), mRNA. /FEA = mRNA |
| | | /GEN = DKFZP547E2110 /PROD = DKFZP547E2110 protein /DB_XREF = gi: 7661591 /UG = Hs.108110 |
| | | DKFZP547E2110 protein /FL = gb: AL050261.1 gb: AF151856.1 gb: AF161495.1 gb: NM_014034.1 gb: |
| 120 | 203455_s_at | gb: NM_002970.1 /DEF = Homo sapiens spermidinespermine N1-acetyltransferase (SAT), mRNA. /FEA = mRNA |
| | | /GEN = SAT /PROD = spermidinespermine N1-acetyltransferase /DB_XREF = gi: 4506788 /UG = Hs.28491 |
| | | spermidinespermine N1-acetyltransferase /FL = gb: BC002503.1 gb: M77693.1 g |
| 121 | 203494_s_at | gb: NM_014679.1 /DEF = Homo sapiens KIAA0092 gene product (KIAA0092), mRNA. /FEA = mRNA /GEN = KIAA0092 |
| | | /PROD = KIAA0092 gene product /DB_XREF = gi: 7661899 /UG = Hs.151791 KIAA0092 gene product /FL = gb: |
| | | D42054.1 gb: NM_014679.1 |
| 122 | 203640_at | Consensus includes gb: BE328496 /FEA = EST /DB_XREF = gi: 9202272 /DB_XREF = est: hs98f09.x1 |
| | | /CLONE = IMAGE: 3145289 /UG = Hs.283609 hypothetical protein PRO2032 /FL = gb: AF116683.1 gb: NM_018615.1 |
| 123 | 203739_at | gb: NM_006526.1 /DEF = Homo sapiens zinc finger protein 217 (ZNF217), mRNA. /FEA = mRNA /GEN = ZNF217 |
| | | /PROD = zinc finger protein 217 /DB_XREF = gi: 5730123 /UG = Hs.155040 zinc finger protein 217 /FL = gb: AF041259.1 |
| | | gb: NM_006526.1 |
| 124 | 203765_at | gb: NM_012198.1 /DEF = Homo sapiens grancalcin (GCL), mRNA. /FEA = mRNA /GEN = GCL /PROD = grancalcin |
| | | /DB_XREF = gi: 6912387 /UG = Hs.79381 grancalcin /FL = gb: BC005214.1 gb: M81637.1 gb: NM_012198.1 |
| 125 | 203799_at | gb: NM_014880.1 /DEF = Homo sapiens KIAA0022 gene product (KIAA0022), mRNA. /FEA = mRNA /GEN = KIAA0022 |
| | | /PROD = KIAA0022 gene product /DB_XREF = gi: 7661867 /UG = Hs.2441 KIAA0022 gene product /FL = gb: D14664.1 |
| | | gb: NM_014880.1 |
| 126 | 203964_at | gb: NM_004688.1 /DEF = Homo sapiens N-myc (and STAT) interactor (NMI), mRNA. /FEA = mRNA /GEN = NMI |
| | | /PROD = N-myc and STAT interactor /DB_XREF = gi: 4758813 /UG = Hs.54483 N-myc (and STAT) interactor |
| | | /FL = gb: BC001268.1 gb: U32849.1 gb: NM_004688.1 |
| 127 | 203983_at | gb: NM_005999.1 /DEF = Homo sapiens translin-associated factor X (TSNAX), mRNA. /FEA = mRNA /GEN = TSNAX |
| | | /PROD = translin-associated factor X /DB_XREF = gi: 5174730 /UG = Hs.96247 translin-associated factor X |
| | | /FL = gb: NM_005999.1 |
| 128 | 204112_s_at | gb: NM_006895.1 /DEF = Homo sapiens histamine N-methyltransferase (HNMT), mRNA. /FEA = mRNA /GEN = HNMT |
| | | /PROD = histamine N-methyltransferase /DB_XREF = gi: 5901969 /UG = Hs.81182 histamine N-methyltransferase |
| | | /FL = gb: U08092.1 gb: D16224.1 gb: NM_006895.1 |
| 129 | 204172_at | gb: NM_000097.1 /DEF = Homo sapiens coproporphyrinogen oxidase (coproporphyria, harderoporphyria) (CPO), mRNA. |
| | | /FEA = mRNA /GEN = CPO /PROD = coproporphyrinogen oxidase (coproporphyria, harderoporphyria) |
| | | /DB_XREF = gi: 4503016 /UG = Hs.89866 coproporphyrinogen oxidase |
| 130 | 204185_x_at | gb: NM_005038.1 /DEF = Homo sapiens peptidylprolyl isomerase D (cyclophilin D) (PPID), mRNA. /FEA = mRNA |
| | | /GEN = PPID /PROD = peptidylprolyl isomerase D (cyclophilin D) /DB_XREF = gi: 4826931 /UG = Hs.143482 |
| | | peptidylprolyl isomerase D (cyclophilin D) /FL = gb: L11667.1 gb |
| 131 | 204194_at | gb: NM_001186.1 /DEF = Homo sapiens BTB and CNC homology 1, basic leucine zipper transcription factor 1 (BACH1), |
| | | mRNA. /FEA = mRNA /GEN = BACH1 /PROD = BTB and CNC homology 1, basic leucine zippertranscription factor 1 |
| | | /DB_XREF = gi: 4502352 /UG = Hs.154276 BTB and CN |
| 132 | 204224_s_at | gb: NM_000161.1 /DEF = Homo sapiens GTP cyclohydrolase 1 (dopa-responsive dystonia) (GCH1), mRNA. /FEA = mRNA |
| | | /GEN = GCH1 /PROD = GTP cyclohydrolase 1 (dopa-responsive dystonia) /DB_XREF = gi: 4503948 /UG = Hs.86724 GTP |
| | | cyclohydrolase 1 (dopa-responsive dystonia) /FL = |
| 133 | 204258_at | gb: NM_001270.1 /DEF = Homo sapiens chromodomain helicase DNA binding protein 1 (CHD1), mRNA. /FEA = mRNA |
| | | /GEN = CHD1 /PROD = chromodomain helicase DNA binding protein 1 /DB_XREF = gi: 4557446 /UG = Hs.22670 |
| | | chromodomain helicase DNA binding protein 1 /FL = gb: AF006513. |
| 134 | 204369_at | gb: NM_006218.1 /DEF = Homo sapiens phosphoinositide-3-kinase, catalytic, alpha polypeptide (PIK3CA), mRNA. |
| | | /FEA = mRNA /GEN = PIK3CA /PROD = phosphoinositide-3-kinase, catalytic, alphapolypeptide /DB_XREF = gi: 5453891 |
| | | /UG = Hs.85701 phosphoinositide-3-kinase, cataly |
| 135 | 204417_at | gb: NM_000153.1 /DEF = Homo sapiens galactosylceramidase (Krabbe disease) (GALC), mRNA. /FEA = mRNA |
| | | /GEN = GALC /PROD = galactosylceramidase precursor /DB_XREF = gi: 4557612 /UG = Hs.273 galactosylceramidase |
| | | (Krabbe disease) /FL = gb: L23116.1 gb: NM_000153.1 gb: D25283.1 |
| 136 | 204615_x_at | gb: NM_004508.1 /DEF = Homo sapiens isopentenyl-diphosphate delta isomerase (IDI1), mRNA. /FEA = mRNA |
| | | /GEN = IDI1 /PROD = isopentenyl-diphosphate delta isomerase /DB_XREF = gi: 4758583 /UG = Hs.76038 isopentenyl- |
| | | diphosphate delta isomerase /FL = gb: NM_004508.1 |
| 137 | 204646_at | gb: NM_000110.2 /DEF = Homo sapiens dihydropyrimidine dehydrogenase (DPYD), mRNA. /FEA = mRNA |
| | | /GEN = DPYD /PROD = dihydropyrimidine dehydrogenase /DB_XREF = gi: 4557874 /UG = Hs.1602 dihydropyrimidine |
| | | dehydrogenase /FL = gb: U20938.1 gb: NM_000110.2 gb: U09178.1 gb: AB003063 |
| 138 | 204748_at | gb: NM_000963.1 /DEF = Homo sapiens prostaglandin-endoperoxide synthase 2 (prostaglandin GH synthase and |
| | | cyclooxygenase) (PTGS2), mRNA. /FEA = mRNA /GEN = PTGS2 /PROD = prostaglandin-endoperoxide synthase |
| | | 2(prostaglandin GH synthase and cyclooxygenase) /DB_XREF = g |
| 139 | 204759_at | gb: NM_001268.1 /DEF = Homo sapiens chromosome condensation 1-like (CHC1L), mRNA. /FEA = mRNA |
| | | /GEN = CHC1L /PROD = RCC1-like G exchanging factor RLG /DB_XREF = gi: 4557444 /UG = Hs.27007 chromosome |
| | | condensation 1-like /FL = gb: AF060219.1 gb: NM_001268.1 |
| 140 | 204780_s_at | Consensus includes gb: AA164751 /FEA = EST /DB_XREF = gi: 1740929 /DB_XREF = est: zo93g12.s1 |
| | | /CLONE = IMAGE: 594502 /UG = Hs.82359 tumor necrosis factor receptor superfamily, member 6 /FL = gb: M67454.1 |
| | | gb: NM_000043.1 |
| 141 | 205062_x_at | gb: NM_002892.2 /DEF = Homo sapiens retinoblastoma-binding protein 1 (RBBP1), transcript variant 1, mRNA. |
| | | /FEA = mRNA /GEN = RBBP1 /PROD = retinoblastoma-binding protein 1, isoform I /DB_XREF = gi: 13259496 |
| | | /UG = Hs.91797 retinoblastoma-binding protein 1 /FL = gb: NM_002 |
| 142 | 205091_x_at | gb: NM_002907.1 /DEF = Homo sapiens RecQ protein-like (DNA helicase Q1-like) (RECQL), mRNA. /FEA = mRNA |
| | | /GEN = RECQL /PROD = RecQ protein-like (DNA helicase Q1-like) /DB_XREF = gi: 4506468 /UG = Hs.235069 |
| | | RecQ protein-like (DNA helicase Q1-like) /FL = gb: NM_002907.1 gb: |
| 143 | 205173_x_at | gb: NM_001779.1 /DEF = Homo sapiens CD58 antigen, (lymphocyte function-associated antigen 3) (CD58), mRNA. |
| | | /FEA = mRNA /GEN = CD58 /PROD = CD58 antigen, (lymphocyte function-associatedantigen 3) /DB_XREF = gi: 4502676 |
| | | /UG = Hs.75626 CD58 antigen, (lymphocyte function- |
| 144 | 205191_at | gb: NM_006915.1 /DEF = Homo sapiens retinitis pigmentosa 2 (X-linked recessive) (RP2), mRNA. /FEA = mRNA |
| | | /GEN = RP2 /PROD = XRP2 protein /DB_XREF = gi: 5902059 /UG = Hs.44766 retinitis pigmentosa 2 (X-linked recessive) |
| | | /FL = gb: NM_006915.1 |
| 145 | 205214_at | gb: NM_004226.1 /DEF = Homo sapiens serinethreonine kinase 17b (apoptosis-inducing) (STK17B), mRNA. /FEA = mRNA |
| | | /GEN = STK17B /PROD = serinethreonine kinase 17b(apoptosis-inducing) /DB_XREF = gi: 4758193 /UG = Hs.120996 |
| | | serinethreonine kinase 17b (apoptosis-inducing) |
| 146 | 205842_s_at | gb: AF001362.1 /DEF = Homo sapiens Jak2 kinase (JAK2) mRNA, complete cds. /FEA = mRNA /GEN = JAK2 |
| | | /PROD = Jak2 kinase /DB_XREF = gi: 3236321 /UG = Hs.115541 Janus kinase 2 (a protein tyrosine kinase) /FL = gb: |
| | | NM_004972.2 gb: AF005216.1 gb: AF058925.1 gb: AF001362.1 |
| 147 | 206003_at | gb: NM_014645.1 /DEF = Homo sapiens KIAA0635 gene product (KIAA0635), mRNA. /FEA = mRNA /GEN = KIAA0635 |
| | | /PROD = KIAA0635 gene product /DB_XREF = gi: 7662215 /UG = Hs.185091 KIAA0635 gene product /FL = gb: |
| | | AB014535.1 gb: NM_014645.1 |
| 148 | 206060_s_at | gb: NM_015967.1 /DEF = Homo sapiens protein tyrosine phosphatase, non-receptor type 22 (lymphoid) (PTPN22), mRNA. |
| | | /FEA = mRNA /GEN = PTPN22 /PROD = protein tyrosine phosphatase homolog /DB_XREF = gi: 7706279 |
| | | /UG = Hs.87860 protein tyrosine phosphatase, non-receptor ty |
| 149 | 206095_s_at | gb: NM_006625.2 /DEF = Homo sapiens TLS-associated serine-arginine protein 1 (TASR1), mRNA. /FEA = mRNA |
| | | /GEN = TASR1 /PROD = TLS-associated serine-arginine protein 1 /DB_XREF = gi: 12056474 /UG = Hs.288038 TLS- |
| | | associated serine-arginine protein 1 /FL = gb: NM_006625.2 |
| 150 | 206158_s_at | gb: NM_003418.1 /DEF = Homo sapiens zinc finger protein 9 (a cellular retroviral nucleic acid binding protein) (ZNF9), |
| | | mRNA. /FEA = mRNA /GEN = ZNF9 /PROD = zinc finger protein 9 (a cellular retroviralnucleic acid binding protein) |
| | | /DB_XREF = gi: 4827070 /UG = Hs.2110 |
| 151 | 206488_s_at | gb: NM_000072.1 /DEF = Homo sapiens CD36 antigen (collagen type I receptor, thrombospondin receptor) (CD36), |
| | | mRNA. /FEA = mRNA /GEN = CD36 /PROD = CD36 antigen (collagen type I receptor, thrombospondin receptor) |
| | | /DB_XREF = gi: 4557418 /UG = Hs.75613 CD36 antigen (colla |
| 152 | 206584_at | gb: NM_015364.1 /DEF = Homo sapiens MD-2 protein (MD-2), mRNA. /FEA = mRNA /GEN = MD-2 /PROD = MD-2 |
| | | protein /DB_XREF = gi: 7662503 /UG = Hs.69328 MD-2 protein /FL = gb: AB018549.1 gb: NM_015364.1 |
| | | gb: AF168121.1 |
| 153 | 206854_s_at | gb: NM_003188.1 /DEF = Homo sapiens mitogen-activated protein kinase kinase kinase 7 (MAP3K7), mRNA. |
| | | /FEA = mRNA /GEN = MAP3K7 /PROD = mitogen-activated protein kinase kinase kinase7 /DB_XREF = gi: 4507360 |
| | | /UG = Hs.7510 mitogen-activated protein kinase kinase kinase 7 |
| 154 | 207387_s_at | gb: NM_000167.1 /DEF = Homo sapiens glycerol kinase (GK), mRNA. /FEA = mRNA /GEN = GK |
| | | /PROD = glycerol kinase /DB_XREF = gi: 4504006 /UG = Hs.1466 glycerol kinase /FL = gb: L13943.1 gb: |
| | | NM_000167.1 |
| 155 | 207785_s_at | gb: NM_015874.1 /DEF = Homo sapiens H-2K binding factor-2 (LOC51580), mRNA. /FEA = mRNA /GEN = LOC51580 |
| | | /PROD = H-2K binding factor-2 /DB_XREF = gi: 7706215 /UG = Hs.327138 H-2K binding factor-2 /FL = gb: D14041.1 |
| | | gb: NM_015874.1 |
| 156 | 207941_s_at | gb: NM_004902.1 /DEF = Homo sapiens splicing factor (CC1.3) (CC1.3), mRNA. /FEA = mRNA /GEN = CC1.3 |
| | | /PROD = splicing factor (CC1.3) /DB_XREF = gi: 4757925 /UG = Hs.145696 splicing factor (CC1.3) /FL = gb: L10910.1 |
| | | gb: NM_004902.1 |
| 157 | 207956_x_at | gb: NM_015928.1 /DEF = Homo sapiens androgen-induced prostate proliferative shutoff associated protein (AS3), mRNA. |
| | | /FEA = mRNA /GEN = AS3 /PROD = androgen-induced prostate proliferative shutoffassociated protein |
| | | /DB_XREF = gi: 7705287 /UG = Hs.168625 androgen-induced |
| 158 | 208296_x_at | gb: NM_014350.1 /DEF = Homo sapiens TNF-induced protein (GG2-1), mRNA. /FEA = mRNA /GEN = GG2-1 |
| | | /PROD = TNF-induced protein /DB_XREF = gi: 7657123 /UG = Hs.17839 TNF-induced protein /FL = gb: AF099936.1 gb: |
| | | NM_014350.1 |
| 159 | 208319_s_at | gb: NM_006743.1 /DEF = Homo sapiens RNA binding motif protein 3 (RBM3), mRNA. /FEA = mRNA /GEN = RBM3 |
| | | /PROD = RNA binding motif protein 3 /DB_XREF = gi: 5803136 /UG = Hs.301404 RNA binding motif protein 3 |
| | | /FL = gb: NM_006743.1 gb: U28686.1 |
| 160 | 208374_s_at | gb: NM_006135.1 /DEF = Homo sapiens capping protein (actin filament) muscle Z-line, alpha 1 (CAPZA1), mRNA. |
| | | /FEA = mRNA /GEN = CAPZA1 /PROD = F-actin capping protein alpha-1 subunit /DB_XREF = gi: 5453596 |
| | | /UG = Hs.184270 capping protein (actin filament) muscle Z-line, |
| 161 | 208398_s_at | gb: NM_004865.1 /DEF = Homo sapiens TBP-like 1 (TBPL1), mRNA. /FEA = mRNA /GEN = TBPL1 |
| | | /PROD = TBP-like 1 /DB_XREF = gi: 4759233 /UG = Hs.13993 TBP-like 1 /FL = gb: AF130312.1 gb: NM_004865.1 |
| 162 | 208405_s_at | gb: NM_006016.1 /DEF = Homo sapiens CD164 antigen, sialomucin (CD164), mRNA. /FEA = mRNA /GEN = CD164 |
| | | /PROD = CD164 antigen, sialomucin /DB_XREF = gi: 5174406 /UG = Hs.43910 CD164 antigen, sialomucin |
| | | /FL = gb: D14043.1 gb: NM_006016.1 |
| 163 | 208669_s_at | gb: AF109873.1 /DEF = Homo sapiens retinoblastoma protein-associated protein mRNA, complete cds. /FEA = mRNA |
| | | /PROD = retinoblastoma protein-associated protein /DB_XREF = gi: 11415000 /UG = Hs.75847 CREBBPEP300 inhibitory |
| | | protein 1 /FL = gb: AF109873.1 gb: AF274947.1 gb: |
| 164 | 208671_at | gb: AF164794.1 /DEF = Homo sapiens Diff33 protein homolog mRNA, complete cds. /FEA = mRNA |
| | | /PROD = Diff33 protein homolog /DB_XREF = gi: 8895090 /UG = Hs.146668 KIAA1253 protein /FL = gb: AF164794.1 |
| 165 | 208673_s_at | gb: AF107405.1 /DEF = Homo sapiens pre-mRNA splicing factor (SFRS3) mRNA, complete cds. /FEA = mRNA |
| | | /GEN = SFRS3 /PROD = pre-mRNA splicing factor /DB_XREF = gi: 5531903 /UG = Hs.167460 splicing |
| | | factor, arginineserine-rich 3 /FL = gb: BC000914.1 gb: AF107405.1 |
| 166 | 208697_s_at | gb: BC000734.1 /DEF = Homo sapiens, eukaryotic translation initiation factor 3, subunit 6 (48 kD), clone MGC: |
| | | 2060, mRNA, complete cds. /FEA = mRNA /PROD = eukaryotic translation initiation factor 3, subunit 6 (48 kD) |
| | | /DB_XREF = gi: 12653884 /UG = Hs.106673 eukaryotic t |
| 167 | 208706_s_at | Consensus includes gb: AK026933.1 /DEF = Homo sapiens cDNA: FLJ23280 fis, clone HEP07194. /FEA = mRNA |
| | | /DB_XREF = gi: 10439907 /UG = Hs.286236 eukaryotic translation initiation factor 5 /FL = gb: AL080102.1 |
| 168 | 208708_x_at | gb: AL080102.1 /DEF = Homo sapiens mRNA; cDNA DKFZp564N1916 (from clone DKFZp564N1916); complete cds. |
| | | /FEA = mRNA /GEN = DKFZp564N1916 /PROD = hypothetical protein /DB_XREF = gi: 5262526 |
| | | /UG = Hs.286236 eukaryotic translation initiation factor 5 /FL = gb: AL080102.1 |
| 169 | 208737_at | gb: BC003564.1 /DEF = Homo sapiens, ATPase, H+ transporting, lysosomal (vacuolar proton pump), member J, clone |
| | | MGC: 1970, mRNA, complete cds. /FEA = mRNA /PROD = ATPase, H+ transporting, lysosomal (vacuolarproton pump), |
| | | member J /DB_XREF = gi: 13097719 /UG = Hs.90336 |
| 170 | 208775_at | gb: D89729.1 /DEF = Homo sapiens mRNA for CRM1 protein, complete cds. /FEA = mRNA /PROD = CRM1 protein |
| | | /DB_XREF = gi: 2626839 /UG = Hs.79090 exportin 1 (CRM1, yeast, homolog) /FL = gb: D89729.1 gb: NM_003400.2 |
| 171 | 208787_at | gb: BC003375.1 /DEF = Homo sapiens, mitochondrial ribosomal protein L3, clone MGC: 5219, mRNA, complete cds. |
| | | /FEA = mRNA /PROD = mitochondrial ribosomal protein L3 /DB_XREF = gi: 13097224 /UG = Hs.79086 mitochondrial |
| | | ribosomal protein L3 /FL = gb: BC003375.1 gb: NM_00720 |
| 172 | 208796_s_at | gb: BC000196.1 /DEF = Homo sapiens, cyclin G1, clone MGC: 643, mRNA, complete cds. /FEA = mRNA |
| | | /PROD = cyclin G1 /DB_XREF = gi: 12652880 /UG = Hs.79101 cyclin G1 /FL = gb: L49504.1 gb: U47413.1 gb: |
| | | BC000196.1 gb: D78341.1 gb: NM_004060.2 |
| 173 | 208808_s_at | gb: BC000903.1 /DEF = Homo sapiens, high-mobility group (nonhistone chromosomal) protein 2, clone MGC: 5234, mRNA, |
| | | complete cds. /FEA = mRNA /PROD = high-mobility group (nonhistone chromosomal)protein 2 /DB_XREF = gi: 12654170 |
| | | /UG = Hs.80684 high-mobility group (nonh |
| 174 | 208841_s_at | gb: AB014560.1 /DEF = Homo sapiens mRNA for KIAA0660 protein, complete cds. /FEA = mRNA /GEN = KIAA0660 |
| | | /PROD = KIAA0660 protein /DB_XREF = gi: 3327133 /UG = Hs.6727 Ras-GTPase activating protein SH3 domain-binding |
| | | protein 2 /FL = gb: AB014560.1 |
| 175 | 208925_at | gb: AF161522.1 /DEF = Homo sapiens HSPC174 mRNA, complete cds. /FEA = mRNA /PROD = HSPC174 |
| | | /DB_XREF = gi: 6841567 /UG = Hs.107393 chromosome 3 open reading frame 4 /FL = gb: NM_019895.1 gb: AF161522.1 |
| 176 | 208943_s_at | gb: U93239.1 /DEF = Human Sec62 (Sec62) mRNA, complete cds. /FEA = mRNA /GEN = Sec62 /PROD = Sec62 |
| | | /DB_XREF = gi: 1928972 /UG = Hs.8146 translocation protein 1 /FL = gb: D87127.1 gb: U93239.1 gb: NM_003262.1 |
| 177 | 209004_s_at | gb: AF142481.1 /DEF = Homo sapiens F-box protein FLR1 (FLR1) mRNA, complete cds. /FEA = mRNA /GEN = FLR1 |
| | | /PROD = F-box protein FLR1 /DB_XREF = gi: 7672733 /UG = Hs.5548 f-box and leucine-rich repeat protein 5 |
| | | /FL = gb: AF199420.1 gb: AF142481.1 gb: AF157323.1 |
| 178 | 209027_s_at | Consensus includes gb: BF673013 /FEA = EST /DB_XREF = gi: 11946908 /DB_XREF = est: 602152905F1 |
| | | /CLONE = IMAGE: 4293985 /UG = Hs.24752 spectrin SH3 domain binding protein 1 /FL = gb: AF006516.1 |
| 179 | 209028_s_at | gb: AF006516.1 /DEF = Homo sapiens eps8 binding protein e3B1 mRNA, complete cds. /FEA = mRNA /PROD = e3B1 |
| | | /DB_XREF = gi: 2245670 /UG = Hs.24752 spectrin SH3 domain binding protein 1 /FL = gb: AF006516.1 |
| 180 | 209033_s_at | gb: D86550.1 /DEF = Human mRNA for serinethreonine protein kinase, complete cds. /FEA = mRNA /GEN = hMNB |
| | | /PROD = serinethreonine protein kinase /DB_XREF = gi: 1772437 /UG = Hs.75842 dual-specificity tyrosine-(Y)- |
| | | phosphorylation regulated kinase 1A /FL = gb: D85759.1 gb: D8 |
| 181 | 209115_at | gb: AL117566.1 /DEF = Homo sapiens mRNA; cDNA DKFZp566J164 (from clone DKFZp566J164); complete cds. |
| | | /FEA = mRNA /GEN = DKFZp566J164 /PROD = hypothetical protein /DB_XREF = gi: 5912116 /UG = Hs.154320 |
| | | ubiquitin-activating enzyme E1C (homologous to yeast UBA3) /FL = gb: AF |
| 182 | 209206_at | Consensus includes gb: AV701283 /FEA = EST /DB_XREF = gi: 10717613 /DB_XREF = est: AV701283 |
| | | /CLONE = ADAAGD06 /UG = Hs.50785 SEC22, vesicle trafficking protein (S. cerevisiae)-like 1 /FL = gb: BC001364.1 |
| | | gb: AF047442.1 gb: NM_004892.1 |
| 183 | 209240_at | Consensus includes gb: AF070560.1 /DEF = Homo sapiens clone 24689 mRNA sequence. /FEA = mRNA |
| | | /DB_XREF = gi: 3387927 /UG = Hs.100293 O-linked N-acetylglucosamine (GlcNAc) transferase (UDP-N- |
| | | acetylglucosamine: polypeptide-N-acetylglucosaminyl transferase /FL = gb: AF223 |
| 184 | 209259_s_at | gb: AF020043.1 /DEF = Homo sapiens chromosome-associated polypeptide (HCAP) mRNA, complete cds. /FEA = mRNA |
| | | /GEN = HCAP /PROD = chromosome-associated polypeptide /DB_XREF = gi: 3089367 /UG = Hs.24485 chondroitin sulfate |
| | | proteoglycan 6 (bamacan) /FL = gb: AF020043.1 gb: NM |
| 185 | 209284_s_at | Consensus includes gb: AI922509 /FEA = EST /DB_XREF = gi: 5658473 /DB_XREF = est: wh13g11.x1 |
| | | /CLONE = IMAGE: 2380676 /UG = Hs.23440 KIAA1105 protein /FL = gb: AF180425.2 |
| 186 | 209300_s_at | gb: BC002888.1 /DEF = Homo sapiens, Similar to DKFZP566B183 protein, clone MGC: 10356, mRNA, complete cds. |
| | | /FEA = mRNA /PROD = Similar to DKFZP566B183 protein /DB_XREF = gi: 12804070 /UG = Hs.12305 DKFZP566B183 |
| | | protein /FL = gb: BC002888.1 |
| 187 | 209308_s_at | gb: BC002461.1 /DEF = Homo sapiens, BCL2adenovirus E1B 19 kD-interacting protein 2, clone MGC: 1529, mRNA, |
| | | complete cds. /FEA = mRNA /PROD = BCL2adenovirus E1B 19 kD-interacting protein 2 /DB_XREF = gi: 12803290 |
| | | /UG=Hs.155596 BCL2adenovirus E1B 19 kD-interacting prote |
| 188 | 209323_at | gb: AF081567.1 /DEF = Homo sapiens death associated protein 4 (DAP4) mRNA, complete cds. /FEA = mRNA |
| | | /GEN = DAP4 /PROD = death associated protein 4 /DB_XREF = gi: 9886758 /UG = Hs.177574 protein-kinase, interferon- |
| | | inducible double stranded RNA dependent inhibitor, repr |
| 189 | 209337_at | gb: AF063020.1 /DEF = Homo sapiens lens epithelium-derived growth factor mRNA, complete cds. /FEA = mRNA |
| | | /PROD = lens epithelium-derived growth factor /DB_XREF = gi: 3283351 /UG = Hs.82110 PC4 and SFRS1 |
| | | interacting protein 1 /FL = gb: NM_021144.1 gb: AF063020.1 |
| 190 | 209348_s_at | gb: AF055376.1 /DEF = Homo sapiens short form transcription factor C-MAF (c-maf) mRNA, complete cds. /FEA = mRNA |
| | | /GEN = c-maf/PROD = short form transcription factor C-MAF /DB_XREF = gi: 3335147 /UG = Hs.30250 v-maf |
| | | musculoaponeurotic fibrosarcoma (avian) oncogene hom |
| 191 | 209404_s_at | gb: AF151867.1 /DEF = Homo sapiens CGI-109 protein mRNA, complete cds. /FEA = mRNA/PROD = CGI-109 protein |
| | | /DB_XREF = gi: 4929686 /UG = Hs.278391 CGI-109 protein /FL = gb: AF151867.1 |
| 192 | 209451_at | gb: U59863.1 /DEF = Human TRAF-interacting protein I-TRAF mRNA, complete cds. /FEA = mRNA /PROD = I-TRAF |
| | | /DB_XREF = gi: 1518017 /UG = Hs.146847 TRAF family member-associated NFKB activator /FL = gb: U59863.1 |
| 193 | 209476_at | Consensus includes gb: AL080080.1 /DEF = Homo sapiens mRNA; cDNA DKFZp564E1962 (from clone |
| | | DKFZp564E1962); partial cds. /FEA = mRNA /GEN = DKFZp564E1962 /PROD = hypothetical protein |
| | | /DB_XREF = gi: 5262491 /UG = Hs.24766 thioredoxin-related transmembrane protein /FL = gb |
| 194 | 209495_at | gb: AF022655.1 /DEF = Homo sapiens cep250 centrosome associated protein mRNA, complete cds. /FEA = mRNA |
| | | /PROD = cep250 centrosome associated protein /DB_XREF = gi: 2832236 /UG = Hs.27910 centrosomal protein 2 |
| | | /FL = gb: AF022655.1 gb: AF049105.1 gb: NM_007186.1 |
| 195 | 209513_s_at | gb: BC004331.1 /DEF = Homo sapiens, Similar to RIKEN cDNA 2610207I16 gene, clone MGC: 10940, mRNA, complete |
| | | cds. /FEA = mRNA/PROD = Similar to RIKEN cDNA 2610207I16 gene /DB_XREF = gi: 13279253 /UG = Hs.47986 Homo |
| | | sapiens, Similar to RIKEN cDNA 2610207I16 gene, clon |
| 196 | 209572_s_at | gb: AF080227.1 /DEF = Homo sapiens embryonic ectoderm development protein mRNA, complete cds. /FEA = mRNA |
| | | /PROD = embryonic ectoderm development protein /DB_XREF = gi: 3420789 /UG = Hs.151461 embryonic ectoderm |
| | | development /FL = gb: AF080227.1 gb: U90651.1 gb: AF078933.1 |
| 197 | 209748_at | gb: AB029006.1 /DEF = Homo sapiens mRNA for KIAA1083 protein, complete cds. /FEA = mRNA /GEN = KIAA1083 |
| | | /PROD = KIAA1083 protein /DB_XREF = gi: 5689502 /UG = Hs.26334 spastic paraplegia 4 (autosomal dominant; spastin) |
| | | /FL = gb: AB029006.1 |
| 198 | 209829_at | gb: AB002384.1 /DEF = Human mRNA for KIAA0386 gene, complete cds. /FEA = mRNA /GEN = KIAA0386 |
| | | /DB_XREF = gi: 2224712 /UG = Hs.101359 chromosome 6 open reading frame 32 /FL = gb: AB002384.1 |
| 199 | 210260_s_at | gb: BC005352.1 /DEF = Homo sapiens, TNF-induced protein, clone MGC: 12451, mRNA, complete cds. /FEA = mRNA |
| | | /PROD = TNF-induced protein /DB_XREF = gi: 13529163 /UG = Hs.17839 TNF-induced protein /FL = gb: BC005352.1 |
| | | gb: AF099935.1 |
| 200 | 210296_s_at | gb: BC005375.1 /DEF = Homo sapiens, peroxisomal membrane protein 3 (35 kD, Zellweger syndrome), clone MGC: 12491, |
| | | mRNA, complete cds. /FEA = mRNA /PROD = peroxisomal membrane protein 3 (35 kD, Zellwegersyndrome) |
| | | /DB_XREF = gi: 13529226 /UG = Hs.180612 peroxisomal membr |
| 201 | 210346_s_at | gb: AF212224.1 /DEF = Homo sapiens CLK4 mRNA, complete cds. /FEA = mRNA /PROD = CLK4 /DB_XREF = |
| | | gi: 9437514 /UG = Hs.295231 Homo sapiens CLK4 mRNA, complete cds /FL = gb: AF212224.1 |
| 202 | 210538_s_at | gb: U37546.1 /DEF = Human IAP homolog C (MIHC) mRNA, complete cds. /FEA = mRNA /GEN = MIHC |
| | | /PROD = MIHC /DB_XREF = gi: 1145290 /UG = Hs.127799 baculoviral IAP repeat-containing 3 /FL = gb: U37546.1 |
| 203 | 210681_s_at | gb: AF153604.1 /DEF = Homo sapiens ubiquitin-specific protease homolog (UPH) mRNA, complete cds. /FEA = mRNA |
| | | /GEN = UPH /PROD = ubiquitin-specific protease homolog /DB_XREF = gi: 5231132 /UG = Hs.23168 ubiquitin specific |
| | | protease 15 /FL = gb: AF153604.1 |
| 204 | 211711_s_at | gb: BC005821.1 /DEF = Homo sapiens, phosphatase and tensin homolog (mutated in multiple advanced cancers 1), clone |
| | | MGC: 11227, mRNA, complete cds. /FEA = mRNA /PROD = phosphatase and tensin homolog (mutated inmultiple |
| | | advanced cancers 1) /DB_XREF = gi: 13543309 /FL |
| 205 | 211943_x_at | Consensus includes gb: AL565449 /FEA = EST /DB_XREF = gi: 12916836 /DB_XREF = est: AL565449 |
| | | /CLONE = CS0DF005YE20 (3 prime) /UG = Hs.279860 tumor protein, translationally-controlled 1 |
| 206 | 212033_at | Consensus includes gb: BF055107 /FEA = EST /DB_XREF = gi: 10809003 /DB_XREF = est: 7j75a05.x1 |
| | | /CLONE = IMAGE: 3392240 /UG = Hs.180789 S164 protein |
| 207 | 212149_at | Consensus includes gb: AW470003 /FEA = EST /DB_XREF = gi: 7040109 /DB_XREF = est: xr27f05.x1 |
| | | /CLONE = IMAGE: 2761377 /UG = Hs.84087 KIAA0143 protein |
| 208 | 212176_at | Consensus includes gb: AA902326 /FEA = EST /DB_XREF = gi: 3037233 /DB_XREF = est: ok92b01.s1 |
| | | /CLONE = IMAGE: 1521385 /UG = Hs.18368 DKFZP564B0769 protein |
| 209 | 212179_at | Consensus includes gb: AW157501 /FEA = EST /DB_XREF = gi: 6228902 /DB_XREF = est: au83a02.x1 |
| | | /CLONE = IMAGE: 2782826 /UG = Hs. 18368 DKFZP564B0769 protein |
| 210 | 212184_s_at | Consensus includes gb: AL117407.1 /DEF = Homo sapiens mRNA; cDNA DKFZp434D2050 (from clone |
| | | DKFZp434D2050); partial cds. /FEA = mRNA /GEN = DKFZp434D2050 /PROD = hypothetical protein |
| | | /DB_XREF = gi: 5911992 /UG = Hs.109727 TAK1-binding protein 2; KIAA0733 protein |
| 211 | 212287_at | Consensus includes gb: BF382924 /FEA = EST /DB_XREF = gi: 11364313 /DB_XREF = est: 601816985F1 |
| | | /CLONE = IMAGE: 4050909 /UG = Hs.197803 KIAA0160 protein |
| 212 | 212289_at | Consensus includes gb: AB020681.1 /DEF = Homo sapiens mRNA for KIAA0874 protein, partial cds. /FEA = mRNA |
| | | /GEN = KIAA0874 /PROD = KIAA0874 protein /DB_XREF = gi: 4240236 /UG = Hs.27973 KIAA0874 protein |
| | | /FL = gb: AF317425.1 |
| 213 | 212306_at | Consensus includes gb: AI741784 /FEA = EST /DB_XREF = gi: 5110072 /DB_XREF = est: wg22h09.x1 |
| | | /CLONE = IMAGE: 2365889 /UG = Hs. 108614 KIAA0627 protein; Drosophila multiple asters (Mast)-like homolog 2 |
| 214 | 212331_at | Consensus includes gb: X76061.1 /DEF = H. sapiens p130 mRNA for 130K protein. /FEA = mRNA /GEN = p130 |
| | | /PROD = 130K protein /DB_XREF = gi: 416030 /UG = Hs.79362 retinoblastoma-like 2 (p130) /FL = gb: NM_005611.1 |
| 215 | 212368_at | Consensus includes gb: AA972711 /FEA = EST /DB_XREF = gi: 3147891 /DB_XREF = est: op90e07.s1 |
| | | /CLONE = IMAGE: 1584132 /UG = Hs.173081 KIAA0530 protein |
| 216 | 212408_at | Consensus includes gb: AK023204.1 /DEF = Homo sapiens cDNA FLJ13142 fis, clone NT2RP3003212, moderately similar |
| | | to Rattus norvegicus lamina associated polypeptide 1C (LAP1C) mRNA. /FEA = mRNA /DB_XREF = gi: 10435025 |
| | | /UG = Hs.234265 DKFZP586G011 protein |
| 217 | 212410_at | Consensus includes gb: AI346431 /FEA = EST /DB_XREF = gi: 4083637 /DB_XREF = est: qp50g09.x1 |
| | | /CLONE = IMAGE: 1926496 /UG = Hs.75188 wee1+ (S. pombe) homolog |
| 218 | 212418_at | Consensus includes gb: M82882.1 /DEF = Human cis-acting sequence. /FEA = mRNA /DB_XREF = gi: 180551 |
| | | /UG = Hs.154365 E74-like factor 1 (ets domain transcription factor) |
| 219 | 212449_s_at | Consensus includes gb: BG288007 /FEA = EST /DB_XREF = gi: 13042412 /DB_XREF = est: 602387785F1 |
| | | /CLONE = IMAGE: 4516701 /UG = Hs.12540 lysophospholipase I |
| 220 | 212450_at | Consensus includes gb: D87445.2 /DEF = Homo sapiens mRNA for KIAA0256 protein, partial cds. /FEA = mRNA |
| | | /GEN = KIAA0256 /PROD = KIAA0256 protein /DB_XREF = gi: 6634006 /UG = Hs.118978 KIAA0256 gene product |
| 221 | 212476_at | Consensus includes gb: D26069.1 /DEF = Human mRNA for KIAA0041 gene, partial cds. /FEA = mRNA |
| | | /GEN = KIAA0041 /DB_XREF = gi: 436227 /UG = Hs.24340 centaurin beta2 |
| 222 | 212536_at | Consensus includes gb: AB023173.1 /DEF = Homo sapiens mRNA for KIAA0956 protein, partial cds. /FEA = mRNA |
| | | /GEN = KIAA0956 /PROD = KIAA0956 protein /DB_XREF = gi: 4589555 /UG = Hs.75478 ATPase, Class VI, type 11B |
| 223 | 212572_at | Consensus includes gb: AW779556 /FEA = EST /DB_XREF = gi: 7794159 /DB_XREF = est: hn81f05.x1 |
| | | /CLONE = IMAGE: 3034305 /UG = Hs.184523 KIAA0965 protein |
| 224 | 212579_at | Consensus includes gb: AB014550.1 /DEF = Homo sapiens mRNA for KIAA0650 protein, partial cds. /FEA = mRNA |
| | | /GEN = KIAA0650 /PROD = KIAA0650 protein /DB_XREF = gi: 3327113 /UG = Hs.8118 KIAA0650 protein |
| 225 | 212588_at | Consensus includes gb: Y00062.1 /DEF = Human mRNA for T200 leukocyte common antigen (CD45, LC-A). |
| | | /FEA = mRNA /DB_XREF = gi: 34275 /UG = Hs.170121 protein tyrosine phosphatase, receptor type, C |
| 226 | 212633_at | Consensus includes gb: AL132776 /DEF = Human DNA sequence from clone RP3-393D12 on chromosome 6q16.1-16.3. |
| | | Contains the 3 end of the gene KIAA0776, the gene for a novel LIM domain protein, ESTs, STSs and GSSs |
| | | /FEA = mRNA_2 /DB_XREF = gi: 6706246 /UG = Hs.5460 KIAA0 |
| 227 | 212779_at | Consensus includes gb: AB029032.1 /DEF = Homo sapiens mRNA for KIAA1109 protein, partial cds. /FEA = mRNA |
| | | /GEN = KIAA1109 /PROD = KIAA1109 protein /DB_XREF = gi: 5689554 /UG = Hs.6606 KIAA1109 protein |
| 228 | 212820_at | Consensus includes gb: AB020663.1 /DEF = Homo sapiens mRNA for KIAA0856 protein, partial cds. /FEA = mRNA |
| | | /GEN = KIAA0856 /PROD = KIAA0856 protein /DB_XREF = gi: 4240200 /UG = Hs.13264 KIAA0856 protein |
| 229 | 212830 at | Consensus includes gb: W68084 /FEA = EST /DB_XREF = gi: 1376954 /DB_XREF = est: zd42f12.s1 /CLONE = IMAGE: |
| | | 343343 /UG = Hs.5599 EGF-like-domain, multiple 5 |
| 230 | 212887_at | Consensus includes gb: AI753659 /FEA = EST /DB_XREF = gi: 5131923 /DB_XREF = est: cr13e03.x1 |
| | | /CLONE = HBMSC_cr13e03 /UG = Hs.321403 Homo sapiens mRNA; cDNA DKFZp564O2363 (from clone |
| | | DKFZp564O2363) |
| 231 | 212893_at | Consensus includes gb: AL080063.1 /DEF = Homo sapiens mRNA; cDNA DKFZp564I052 (from clone DKFZp564I052). |
| | | /FEA = mRNA /GEN = DKFZp564I052 /PROD = hypothetical protein /DB_XREF = gi: 5262468 /UG = Hs.5364 |
| | | DKFZP564I052 protein |
| 232 | 212956_at | Consensus includes gb: AI348094 /FEA = EST /DB_XREF = gi: 4085300 /DB_XREF = est: qp61g12.x1 |
| | | /CLONE = IMAGE: 1927558 /UG = Hs.90419 KIAA0882 protein |
| 233 | 212982_at | Consensus includes gb: AI621223 /FEA = EST /DB_XREF = gi: 4630349 /DB_XREF = est: ts77a09.x1 |
| | | /CLONE = IMAGE: 2237272 /UG = Hs.4014 KIAA0946 protein; Huntingtin interacting protein H |
| 234 | 213000_at | Consensus includes gb: AP000693 /DEF = Homo sapiens genomic DNA, chromosome 21q22.2, PCR fragment from BAC |
| | | clone: KB739C11, CBR1-HLCS region /FEA = mRNA /DB_XREF = gi: 6693637 /UG = Hs.70359 KIAA0136 protein |
| 235 | 213044_at | Consensus includes gb: N22548 /FEA = EST /DB_XREF = gi: 1128682 /DB_XREF = est: yw30c06.s1 |
| | | /CLONE = IMAGE: 253738 /UG = Hs.17820 Rho-associated, coiled-coil containing protein kinase 1 |
| 236 | 213111_at | Consensus includes gb: AB023198.1 /DEF = Homo sapiens mRNA for KIAA0981 protein, partial cds. /FEA = mRNA |
| | | /GEN = KIAA0981 /PROD = KIAA0981 protein /DB_XREF = gi: 4589605 /UG = Hs.158135 KIAA0981 protein |
| 237 | 213151_s_at | Consensus includes gb: AU157515 /FEA = EST /DB_XREF = gi: 11019036 /DB_XREF = est: AU157515 |
| | | /CLONE = PLACE1008067 /UG = Hs.184326 CDC10 (cell division cycle 10, S. cerevisiae, homolog) |
| 238 | 213168_at | Consensus includes gb: AU145005 /FEA = EST /DB_XREF = gi: 11006526 /DB_XREF = est: AU145005 |
| | | /CLONE = HEMBA1003603 /UG = Hs.44450 Sp3 transcription factor |
| 239 | 213251_at | Consensus includes gb: AV712064 /FEA = EST /DB_XREF = gi: 10731370 /DB_XREF = est: AV72064 |
| | | /CLONE = DCAAUD05 /UG = Hs.9456 SWISNF related, matrix associated, actin dependent regulator of chromatin, |
| | | subfamily a, member 5 |
| 240 | 213404_s_at | Consensus includes gb: BF033683 /FEA = EST /DB_XREF = gi: 10741395 /DB_XREF = est: 601453992F1 |
| | | /CLONE = IMAGE: 3857711 /UG = Hs.279903 Ras homolog enriched in brain 2 |
| 241 | 213618_at | Consensus includes gb: AB011152.1 /DEF = Homo sapiens mRNA for KIAA0580 protein, partial cds. /FEA = mRNA |
| | | /GEN = KIAA0580 /PROD = KIAA0580 protein /DB_XREF = gi: 3043683 /UG = Hs.22572 KIAA0580 protein |
| 242 | 213743_at | Consensus includes gb: BE674119 /FEA = EST /DB_XREF = gi: 10034660 /DB_XREF = est: 7d75b03.x1 |
| | | /CLONE = IMAGE: 3278765 /UG = Hs.155478 cyclin T2 |
| 243 | 213945_s_at | Consensus includes gb: AA909765 /FEA = EST /DB_XREF = gi: 3050564 /DB_XREF = est: ok88d11.s1 |
| | | /CLONE = IMAGE: 1521045 /UG = Hs.56966 KIAA0906 protein |
| 244 | 214173_x_at | Consensus includes gb: AW514900 /FEA = EST /DB_XREF = gi: 7152982 /DB_XREF = est: xu89a06.x1 |
| | | /CLONE = IMAGE: 2808850 /UG = Hs.7943 RPB5-mediating protein |
| 245 | 214429_at | Consensus includes gb: U47635.1 /DEF = Human D13S824E locus mRNA, complete cds. /FEA = mRNA |
| | | /DB_XREF = gi: 1669390 /UG = Hs.79877 myotubularin related protein 6 /FL = gb: U47635.1 |
| 246 | 214467_at | Consensus includes gb: NM_003608.1 /DEF = Homo sapiens G protein-coupled receptor 65 (GPR65), mRNA. /FEA = CDS |
| | | /GEN = GPR65 /PROD = G protein-coupled receptor 65 /DB_XREF = gi: 4507420 /UG = Hs.131924 G protein-coupled |
| | | receptor 65 /FL = gb: NM_003608.1 |
| 247 | 214544_s_at | Consensus includes gb: NM_003825.1 /DEF = Homo sapiens synaptosomal-associated protein, 23 kD (SNAP23), mRNA. |
| | | /FEA = CDS /GEN = SNAP23 /PROD = synaptosomal-associated protein, 23 kD /DB_XREF = gi: 4507096 |
| | | /UG = Hs.184376 synaptosomal-associated protein, 23 kD /FL = gb: Y0956 |
| 248 | 215071_s_at | Consensus includes gb: AL353759 /DEF = Human DNA sequence from clone RP1-221C16 on chromosome 6. Contains |
| | | two genes for novel histone 4 family members, two genes for novel histone 1 family members, three genes for novel |
| | | histone 2B family members, a gene for |
| 249 | 215245_x_at | Consensus includes gb: AA830884 /FEA = EST /DB_XREF = gi: 2903983 /DB_XREF = est: oc57a09.s1 |
| | | /CLONE = IMAGE: 1353784 /UG = Hs.89764 fragile X mental retardation 1 |
| 250 | 215596_s_at | Consensus includes gb: AL163248 /DEF = Homo sapiens chromosome 21 segment HS21C048 /FEA = mRNA_2 |
| | | /DB_XREF = gi: 7717304 /UG = Hs.288773 zinc finger protein 294 |
| 251 | 217734_s_at | gb: NM_018031.2 /DEF = Homo sapiens WD repeat domain 6 (WDR6), mRNA. /FEA mRNA /GEN = WDR6 |
| | | /PROD = WD repeat domain 6 /DB_XREF = gi: 11072092 /UG = Hs.8737 WD repeat domain 6 /FL = gb: NM_018031.2 |
| | | gb: AF099100.1 |
| 252 | 217739_s_at | gb: NM_005746.1 /DEF = Homo sapiens pre-B-cell colony-enhancing factor (PBEF), mRNA. /FEA = mRNA /GEN = PBEF |
| | | /PROD = pre-B-cell colony-enhancing factor /DB_XREF = gi: 5031976 /UG = Hs.239138 pre-B-cell |
| | | colony-enhancing factor /FL = gb: U02020.1 gb: NM_005746.1 |
| 253 | 217745_s_at | gb: NM_025146.1 /DEF = Homo sapiens hypothetical protein FLJ13194 (FLJ13194), mRNA. /FEA = mRNA |
| | | /GEN = FLJ13194 /PROD = hypothetical protein FLJ13194 /DB_XREF = gi: 13376734 /UG = Hs.288932 |
| | | hypothetical protein FLJ13194 /FL = gb: NM_025146.1 |
| 254 | 217773_s_at | gb: NM_002489.1 /DEF = Homo sapiens NADH dehydrogenase (ubiquinone) 1 alpha subcomplex, 4 (9 kD, MLRQ) |
| | | (NDUFA4), mRNA. /FEA = mRNA /GEN = NDUFA4 /PROD = NADH dehydrogenase (ubiquinone) 1 alphasubcomplex, 4 |
| | | (9 kD, MLRQ) /DB_XREF = gi: 4505356 /UG = Hs.50098 NADH dehydrog |
| 255 | 217783_s_at | gb: NM_016061.1 /DEF = Homo sapiens CGI-127 protein (LOC51646), mRNA. /FEA = mRNA /GEN = LOC51646 |
| | | /PROD = CGI-127 protein /DB_XREF = gi: 7706340 /UG = Hs.184542 CGI-127 protein /FL = gb: BC000836.1 |
| | | gb: AF151885.1 gb: NM_016061.1 |
| 256 | 217811_at | gb: NM_016275.1 /DEF = Homo sapiens selenoprotein T (LOC51714), mRNA. /FEA = mRNA /GEN = LOC51714 |
| | | /PROD = selenoprotein T /DB_XREF = gi: 7706470 /UG = Hs.8148 selenoprotein T /FL = gb: AF131856.1 gb: AF195141.1 |
| | | gb: NM_016275.1 |
| 257 | 217816_s_at | gb: NM_020357.1 /DEF = Homo sapiens PEST-containing nuclear protein (pcnp), mRNA. /FEA = mRNA /GEN = pcnp |
| | | /PROD = PEST-containing nuclear protein /DB_XREF = gi: 9966826 /UG = Hs.283728 PEST-containing nuclear protein |
| | | /FL = gb: AB037675.1 gb: NM_020357.1 |
| 258 | 217823_s_at | Consensus includes gb: AL562528 /FEA = EST /DB_XREF = gi: 12911037 /DB_XREF = est: AL562528 |
| | | /CLONE = CS0DC012YI08 (3 prime) /UG = Hs.184325 CGI-76 protein /FL = gb: AF151834.1 gb: AF161502.1 |
| | | gb: AF151039.1 gb: NM_016021.1 |
| 259 | 217845_x_at | gb: NM_014056.1 /DEF = Homo sapiens DKFZP564K247 protein (DKFZP564K247), mRNA. /FEA = mRNA |
| | | /GEN = DKFZP564K247 /PROD = DKFZP564K247 protein /DB_XREF = gi: 7661619 /UG = Hs.7917 |
| | | DKFZP564K247 protein /FL = gb: BC000601.1 gb: AF077034.1 gb: AF145385.1 gb: AL110233.1 gb: NM_01405 |
| 260 | 217852_s_at | gb: NM_018184.1 /DEF = Homo sapiens hypothetical protein FLJ10702 (FLJ10702), mRNA. /FEA = mRNA |
| | | /GEN = FLJ10702 /PROD = hypothetical protein FLJ10702 /DB_XREF = gi: 8922600 /UG = Hs.104222 hypothetical |
| | | protein FLJ10702 /FL = gb: NM_018184.1 |
| 261 | 217870_s_at | gb: NM_016308.1 /DEF = Homo sapiens UMP-CMP kinase (LOC51727), mRNA. /FEA = mRNA /GEN = LOC51727 |
| | | /PROD = UMP-CMP kinase /DB_XREF = gi: 7706496 /UG = Hs.11463 UMP-CMP kinase /FL = gb: AF259961.1 |
| | | gb: AF110643.1 gb: AF112216.1 gb: AF070416.1 gb: NM_016308.1 |
| 262 | 217873_at | gb: NM_016289.1 /DEF = Homo sapiens MO25 protein (LOC51719), mRNA. /FEA = mRNA /GEN = LOC51719 |
| | | /PROD = MO25 protein /DB_XREF = gi: 7706480 /UG = Hs.6406 MO25 protein /FL = gb: AF113536.1 gb: NM_016289.1 |
| 263 | 217941_s_at | gb: NM_018695.1 /DEF = Homo sapiens erbb2-interacting protein ERBIN (LOC55914), mRNA. /FEA = mRNA |
| | | /GEN = LOC55914 /PROD = erbb2-interacting protein ERBIN /DB_XREF = gi: 8923908 /UG = Hs.8117 erbb2-interacting |
| | | protein ERBIN /FL = gb: AF263744.1 gb: NM_018695.1 |
| 264 | 217945_at | gb: NM_025238.1 /DEF = Homo sapiens BTB (POZ) domain containing 1 (BTBD1), mRNA. /FEA = mRNA |
| | | /GEN = BTBD1 /PROD = BTB (POZ) domain containing 1 /DB_XREF = gi: 13376847 /UG = Hs.21332 BTB |
| | | (POZ) domain containing 1 /FL = gb: AL136853.1 gb: AF257241.1 gb: NM_025238.1 gb: AF3554 |
| 265 | 217971_at | gb: NM_021970.1 /DEF = Homo sapiens MEK partner 1 (MP1), mRNA. /FEA = mRNA /GEN = MP1 /PROD = MEK |
| | | partner 1 /DB_XREF = gi: 11496276 /UG = Hs.6361 MEK partner 1 /FL = gb: AF130115.1 gb: NM_021970.1 |
| | | gb: AF201947.1 |
| 266 | 217986_s_at | gb: NM_013448.1 /DEF = Homo sapiens bromodomain adjacent to zinc finger domain, 1A (BAZ1A), mRNA. |
| | | /FEA = mRNA /GEN = BAZ1A /PROD = bromodomain adjacent to zinc finger domain, 1A /DB_XREF = gi: 7304918 |
| | | /UG = Hs.8858 bromodomain adjacent to zinc finger domain, 1A /FL = gb |
| 267 | 217989_at | gb: NM_016245.1 /DEF = Homo sapiens retinal short-chain dehydrogenasereductase retSDR2 (LOC51170), mRNA. |
| | | /FEA = mRNA /GEN = LOC51170 /PROD = retinal short-chain dehydrogenasereductaseretSDR2 /DB_XREF = gi: 7705904 |
| | | /UG = Hs.12150 retinal short-chain dehydrogenasereduct |
| 268 | 218067_s_at | gb: NM_018011.1 /DEF = Homo sapiens hypothetical protein FLJ10154 (FLJ10154), mRNA. /FEA = mRNA |
| | | /GEN = FLJ10154 /PROD = hypothetical protein FLJ10154 /DB_XREF = gi: 8922258 /UG = Hs.179972 |
| | | hypothetical protein FLJ10154 /FL = gb: NM_018011.1 |
| 269 | 218093_s_at | gb: NM_017664.1 /DEF = Homo sapiens hypothetical protein FLJ20093 (FLJ20093), mRNA. /FEA = mRNA |
| | | /GEN = FLJ20093 /PROD = hypothetical protein FLJ20093 /DB_XREF = gi: 8923103 /UG = Hs.172572 |
| | | hypothetical protein FLJ20093 /FL = gb: NM_017664.1 |
| 270 | 218095_s_at | gb: NM_018475.1 /DEF = Homo sapiens uncharacterized hypothalamus protein HTMP (LOC55858), mRNA. /FEA = mRNA |
| | | /GEN = LOC55858 /PROD = uncharacterized hypothalamus protein HTMP /DB_XREF = gi: 8923860 /UG = Hs.236510 |
| | | uncharacterized hypothalamus protein HTMP /FL = gb: BC0035 |
| 271 | 218135_at | gb: NM_016570.1 /DEF = Homo sapiens CDA14 (LOC51290), mRNA. /FEA = mRNA /GEN = LOC51290 |
| | | /PROD = CDA14 /DB_XREF = gi: 7706104. /UG = Hs.26813 CDA14 /FL = gb: BC000887.1 gb: AF216751.1 gb: |
| | | NM_016570.1 gb: AF183410.1 |
| 272 | 218171_at | gb: AF195514.1 /DEF = Homo sapiens VPS4-2 ATPase (VPS42) mRNA, complete cds. /FEA = mRNA /GEN = VPS42 |
| | | /PROD = VPS4-2 ATPase /DB_XREF = gi: 11225484 /UG = Hs.126550 suppressor of K+ transport defect 1 |
| | | /FL = gb: AF195514.1 gb: AF038960.1 gb: NM_004869.1 |
| 273 | 218191_s_at | gb: NM_018368.1 /DEF = Homo sapiens hypothetical protein FLJ11240 (FLJ11240), mRNA. /FEA = mRNA |
| | | /GEN = FLJ11240 /PROD = hypothetical protein FLJ11240 /DB_XREF = gi: 8922955 /UG = Hs.301308 |
| | | hypothetical protein FLJ11240 /FL = gb: AF113224.1 gb: AF211480.1 gb: NM_018368.1 |
| 274 | 218195_at | gb: NM_024573.1 /DEF = Homo sapiens hypothetical protein FLJ12910 (FLJ12910), mRNA. /FEA = mRNA |
| | | /GEN = FLJ12910 /PROD = hypothetical protein FLJ12910 /DB_XREF = gi: 13375745 /UG = Hs.15929 |
| | | hypothetical protein FLJ12910 /FL = gb: NM_024573.1 |
| 275 | 218196_at | gb: NM_014028.1 /DEF = Homo sapiens HSPC019 protein (HSPC019), mRNA. /FEA = mRNA /GEN = HSPC019 |
| | | /PROD = HSPC019 protein /DB_XREF = gi: 7661737 /UG = Hs.163724 HSPC019 protein /FL = gb: AF077205.1 |
| | | gb: NM_014028.1 |
| 276 | 218230_at | Consensus includes gb: AL044651 /FEA = EST /DB_XREF = gi: 5432866 /DB_XREF = est: DKFZp434P082_s1 |
| | | /CLONE = DKFZp434P082 /UG = Hs.301064 arfaptin 1 /FL = gb: U52521.1 gb: NM_014447.1 |
| 277 | 218247_s_at | gb: NM_016626.1 /DEF = Homo sapiens hypothetical protein (LOC51320), mRNA. /FEA = mRNA /GEN = LOC51320 |
| | | /PROD = hypothetical protein /DB_XREF = gi: 7706165 /UG = Hs.12830 hypothetical protein /FL = gb: AF208855.1 |
| | | gb: NM_016626.1 |
| 278 | 218303_x_at | gb: NM_016618.1 /DEF = Homo sapiens hypothetical protein (LOC51315), mRNA. /FEA = mRNA /GEN = LOC51315 |
| | | /PROD = hypothetical protein /DB_XREF = gi: 7706155 /UG = Hs.5721 hypothetical protein /FL = gb: AF208845.1 |
| | | gb: AF217520.1 gb: NM_016618.1 |
| 279 | 218304_s_at | gb: NM_022776.1 /DEF = Homo sapiens hypothetical protein FLJ13164 (FLJ13164), mRNA. /FEA = mRNA |
| | | /GEN = FLJ13164 /PROD = hypothetical protein FLJ13164 /DB_XREF = gi: 12232460 /UG = Hs.61260 |
| | | hypothetical protein FLJ13164 /FL = gb: NM_022776.1 |
| 280 | 218313_s_at | gb: NM_017423.1 /DEF = Homo sapiens UDP-N-acetyl-alpha-D-galactosamine: polypeptide N- |
| | | acetylgalactosaminyltransferase 7 (GalNAc-T7) (GALNT7), mRNA. /FEA = mRNA /GEN = GALNT7 |
| | | /PROD = polypeptide N-acetylgalactosaminyltransferase 7 /DB_XREF = gi: 8393408 /UG = Hs.246315 |
| 281 | 218319_at | gb: NM_020651.2 /DEF = Homo sapiens pellino (Drosophila) homolog 1 (PELI1), mRNA. /FEA = mRNA /GEN = PELI1 |
| | | /PROD = pellino protein /DB_XREF = gi: 11037062 /UG = Hs.7886 pellino (Drosophila) homolog 1 /FL = gb: AF302505.1 |
| | | gb: AF300987.1 gb: NM_020651.2 |
| 282 | 218396_at | gb: NM_017684.1 /DEF = Homo sapiens hypothetical protein FLJ20136 (FLJ20136), mRNA. /FEA = mRNA |
| | | /GEN = FLJ20136 /PROD = hypothetical protein FLJ20136 /DB_XREF = gi: 8923138 /UG = Hs.24817 hypothetical protein |
| | | FLJ20136 /FL = gb: NM_017684.1 |
| 283 | 218423_x_at | gb: NM_016516.1 /DEF = Homo sapiens tumor antigen SLP-8p (HCC8), mRNA. /FEA = mRNA /GEN = HCC8 |
| | | /PROD = tumor antigen SLP-8p /DB_XREF = gi: 7705396 /UG = Hs.48499 tumor antigen SLP-8p /FL = gb: AF102177.1 |
| | | gb: NM_016516.1 |
| 284 | 218499_at | gb: NM_016542.1 /DEF = Homo sapiens serinethreonine protein kinase MASK (LOC51765), mRNA. /FEA = mRNA |
| | | /GEN = LOC51765 /PROD = serinethreonine protein kinase MASK /DB_XREF = gi: 7706568 /UG = Hs.23643 |
| | | serinethreonine protein kinase MASK /FL = gb: AB040057.1 gb: NM_016542.1 |
| 285 | 218518_at | gb: NM_016603.1 /DEF = Homo sapiens GAP-like protein (LOC51306), mRNA. /FEA = mRNA /GEN = LOC51306 |
| | | /PROD = GAP-like protein /DB_XREF = gi: 7706136 /UG = Hs.82035 potential nuclear protein C5ORF5; GAP-like protein |
| | | /FL = gb: AF251038.1 gb: AF157316.1 gb: NM_016603.1 |
| 286 | 218519_at | gb: NM_017945.1 /DEF = Homo sapiens hypothetical protein FLJ20730 (FLJ20730), mRNA. /FEA = mRNA |
| | | /GEN = FLJ20730 /PROD = hypothetical protein FLJ20730 /DB_XREF = gi: 8923656 /UG = Hs.237480 |
| | | hypothetical protein FLJ20730 /FL = gb: BC005207.1 gb: NM_017945.1 |
| 287 | 218577_at | gb: NM_017768.1 /DEF = Homo sapiens hypothetical protein FLJ20331 (FLJ20331), mRNA. /FEA = mRNA |
| | | /GEN = FLJ20331 /PROD = hypothetical protein FLJ20331 /DB_XREF = gi: 8923306 /UG = Hs.50848 hypothetical protein |
| | | FLJ20331 /FL = gb: BC003407.1 gb: NM_017768.1 |
| 288 | 218589_at | gb: NM_005767.1 /DEF = Homo sapiens purinergic receptor (family A group 5) (P2Y5), mRNA. /FEA = mRNA |
| | | /GEN = P2Y5/PROD = purinergic receptor (family A group 5) /DB_XREF = gi: 5031968 /UG = Hs.189999 purinergic |
| | | receptor (family A group 5) /FL = gb: AF000546.1 gb: NM_005767. |
| 289 | 218603_at | gb: NM_016217.1 /DEF = Homo sapiens hHDC for homolog of Drosophila headcase (LOC51696), mRNA. /FEA = mRNA |
| | | /GEN = LOC51696 /PROD = hHDC for homolog of Drosophila headcase /DB_XREF = gi: 7706434 |
| | | /UG = Hs.6679 hHDC for homolog of Drosophila headcase /FL = gb: AB033492.1 gb: |
| 290 | 218614_at | gb: NM_018169.1 /DEF = Homo sapiens hypothetical protein FLJ10652 (FLJ10652), mRNA. /FEA = mRNA |
| | | /GEN = FLJ10652 /PROD = hypothetical protein FLJ10652 /DB_XREF = gi: 8922572 /UG = Hs.236844 |
| | | hypothetical protein FLJ10652 /FL = gb: NM_018169.1 |
| 291 | 218640_s_at | gb: NM_024613.1 /DEF = Homo sapiens hypothetical protein FLJ13187 (FLJ13187), mRNA. /FEA = mRNA |
| | | /GEN = FLJ13187 /PROD = hypothetical protein FLJ13187 /DB_XREF = gi: 13375826 /UG = Hs.29724 |
| | | hypothetical protein FLJ13187 /FL = gb: NM_024613.1 |
| 292 | 218668_s_at | gb: NM_021183.1 /DEF = Homo sapiens hypothetical protein similar to small G proteins, especially RAP-2A (LOC57826), |
| | | mRNA. /FEA = mRNA /GEN = LOC57826 /PROD = hypothetical protein similar to small Gproteins, especially RAP-2A |
| | | /DB_XREF = gi: 10880976 /UG = Hs.225979 hyp |
| 293 | 218669_at | gb: NM_021183.1 /DEF = Homo sapiens hypothetical protein similar to small G proteins, especially RAP-2A (LOC57826), |
| | | mRNA. /FEA = mRNA /GEN = LOC57826 /PROD = hypothetical protein similar to small Gproteins, especially RAP-2A |
| | | /DB_XREF = gi: 10880976 /UG = Hs.225979 hyp |
| 294 | 218713_at | gb: NM_024611.1 /DEF = Homo sapiens hypothetical protein FLJ11896 (FLJ11896), mRNA. /FEA = mRNA |
| | | /GEN = FLJ11896 /PROD = hypothetical protein FLJ11896 /DB_XREF = gi: 13375822 /UG = Hs.29263 |
| | | hypothetical protein FLJ11896 /FL = gb: NM_024611.1 |
| 295 | 218728_s_at | gb: NM_014184.1 /DEF = Homo sapiens HSPC163 protein (HSPC163), mRNA. /FEA = mRNA /GEN = HSPC163 |
| | | /PROD = HSPC163 protein /DB_XREF = gi: 7661823 /UG = Hs.108854 HSPC163 protein /FL = gb: BC000573.1 |
| | | gb: AF161512.1 gb: NM_014184.1 |
| 296 | 218738_s_at | gb: NM_016271.1 /DEF = Homo sapiens STRIN protein (STRIN), mRNA. /FEA = mRNA /GEN = STRIN /PROD = STRIN |
| | | protein /DB_XREF = gi: 7706722 /UG = Hs.180403 STRIN protein /FL = gb: AF162680.3 gb: NM_016271.1 |
| 297 | 218791_s_at | gb: NM_024713.1 /DEF = Homo sapiens hypothetical protein FLJ22557 (FLJ22557), mRNA. /FEA = mRNA |
| | | /GEN = FLJ22557 /PROD = hypothetical protein FLJ22557 /DB_XREF = gi: 13376012 /UG = Hs.106101 hypothetical |
| | | protein FLJ22557 /FL = gb: AL136908.1 gb: NM_024713.1 |
| 298 | 218846_at | gb: NM_004830.1 /DEF = Homo sapiens cofactor required for Sp1 transcriptional activation, subunit 3 (130 kD) (CRSP3), |
| | | mRNA. /FEA = mRNA /GEN = CRSP3 /PROD = cofactor required for Sp1 transcriptionalactivation, subunit 3 (130 kD) |
| | | /DB_XREF = gi: 7019352 /UG = Hs.29679 cof |
| 299 | 218854_at | gb: NM_013352.1 /DEF = Homo sapiens squamous cell carcinoma antigen recognized by T cell (SART-2), mRNA. |
| | | /FEA = mRNA /GEN = SART-2 /PROD = squamous cell carcinoma antigen recognized by Tcell /DB_XREF = gi: 7019520 |
| | | /UG = Hs.58636 squamous cell carcinoma antigen recogni |
| 300 | 218871_x_at | gb: NM_018590.1 /DEF = Homo sapiens hypothetical protein PRO0082 (PRO0082), mRNA. /FEA = mRNA |
| | | /GEN = PRO0082 /PROD = hypothetical protein PRO0082 /DB_XREF = gi: 8923965 /UG = Hs.180758 hypothetical |
| | | protein PRO0082 /FL = gb: AF116646.1 gb: NM_018590.1 |
| 301 | 218878_s_at | gb: NM_012238.3 /DEF = Homo sapiens sirtuin (silent mating type information regulation 2, S. cerevisiae, homolog) 1 |
| | | (SIRT1), mRNA. /FEA = mRNA /GEN = SIRT1 /PROD = sirtuin 1 /DB_XREF = gi: 13775598 /UG = Hs.31176 |
| | | sirtuin (silent mating type information regulation 2, S |
| 302 | 218940_at | gb: NM_024558.1 /DEF = Homo sapiens hypothetical protein FLJ13920 (FLJ13920), mRNA. /FEA = mRNA |
| | | /GEN = FLJ13920 /PROD = hypothetical protein FLJ13920 /DB_XREF = gi: 13375724 /UG = Hs.13056 hypothetical |
| | | protein FLJ13920 /FL = gb: NM_024558.1 |
| 303 | 218962_s_at | gb: NM_022484.1 /DEF = Homo sapiens hypothetical protein FLJ13576 (FLJ13576), mRNA. /FEA = mRNA |
| | | /GEN = FLJ13576 /PROD = hypothetical protein FLJ13576 /DB_XREF = gi: 11968036 /UG = Hs.79353 hypothetical |
| | | protein FLJ13576 /FL = gb: NM_022484.1 |
| 304 | 219025_at | gb: NM_020404.1 /DEF = Homo sapiens tumor endothelial marker 1 precursor (TEM1), mRNA. /FEA = mRNA |
| | | /GEN = TEM1 /PROD = tumor endothelial marker 1 precursor /DB_XREF = gi: 9966884 /UG = Hs.195727 tumor |
| | | endothelial marker 1 precursor /FL = gb: AF279142.1 gb: NM_020404.1 |
| 305 | 219065_s_at | gb: NM_015955.1 /DEF = Homo sapiens CGI-27 protein (LOC51072), mRNA. /FEA = mRNA /GEN = LOC51072 |
| | | /PROD = CGI-27 protein /DB_XREF = gi: 7705719 /UG = Hs.20814 CGI-27 protein /FL = gb: AF132961.1 gb: |
| | | NM_015955.1 |
| 306 | 219069_at | gb: NM_017704.1 /DEF = Homo sapiens hypothetical protein FLJ20189 (FLJ20189), mRNA. /FEA = mRNA |
| | | /GEN = FLJ20189 /PROD = hypothetical protein FLJ20189 /DB_XREF = gi: 8923180 /UG = Hs.29052 hypothetical protein |
| | | FLJ20189 /FL = gb: NM_017704.1 |
| 307 | 219130_at | gb: NM_019083.1 /DEF = Homo sapiens hypothetical protein (FLJ10287), mRNA. /FEA = mRNA /GEN = FLJ10287 |
| | | /PROD = hypothetical protein FLJ11219 /DB_XREF = gi: 11024703 /UG = Hs.40337 hypothetical protein |
| | | /FL = gb: NM_019083.1 |
| 308 | 219286_s_at | gb: NM_022768.1 /DEF = Homo sapiens hypothetical protein FLJ12479 (FLJ12479), mRNA. /FEA = mRNA |
| | | /GEN = FLJ12479 /PROD = hypothetical protein FLJ12479 /DB_XREF = gi: 12232444 /UG = Hs.46670 hypothetical |
| | | protein FLJ12479 /FL = gb: NM_022768.1 |
| 309 | 219356_s_at | gb: NM_016410.1 /DEF = Homo sapiens hypothetical protein (HSPC177), mRNA. /FEA = mRNA /GEN = HSPC177 |
| | | /PROD = hypothetical protein /DB_XREF = gi: 7705488 /UG = Hs.279777 hypothetical protein /FL = gb: AF161525.1 |
| | | gb: NM_016410.1 |
| 310 | 219691_at | gb: NM_017654.1 /DEF = Homo sapiens hypothetical protein FLJ20073 (FLJ20073), mRNA. /FEA = mRNA |
| | | /GEN = FLJ20073 /PROD = hypothetical protein FLJ20073 /DB_XREF = gi: 8923080 /UG = Hs.65641 hypothetical protein |
| | | FLJ20073 /FL = gb: NM_017654.1 |
| 311 | 219960_s_at | gb: NM_015984.1 /DEF = Homo sapiens ubiquitin C-terminal hydrolase UCH37 (UCH37), mRNA. /FEA = mRNA |
| | | /GEN = UCH37 /PROD = ubiquitin C-terminal hydrolase UCH37 /DB_XREF = gi: 7706752 /UG = Hs.171581 ubiquitin C- |
| | | terminal hydrolase UCH37 /FL = gb: AF147717.1 gb: NM_015984.1 |
| 312 | 220547_s_at | gb: NM_019054.1 /DEF = Homo sapiens hypothetical protein MGC5560 (MGC5560), mRNA. /FEA = mRNA |
| | | /GEN = MGC5560 /PROD = hypothetical protein MGC5560 /DB_XREF = gi: 12963480 /UG = Hs.233150 hypothetical |
| | | protein MGC5560 /FL = gb: NM_019054.1 |
| 313 | 220926_s_at | gb: NM_025191.1 /DEF = Homo sapiens hypothetical protein FLJ13033 (FLJ13033), mRNA. /FEA = mRNA |
| | | /GEN = FLJ13033 /PROD = hypothetical protein FLJ13033 /DB_XREF = gi: 13376781 /UG = Hs.301997 hypothetical |
| | | protein FLJ13033 /FL = gb: NM_025191.1 |
| 314 | 220933_s_at | gb: NM_024617.1 /DEF = Homo sapiens hypothetical protein FLJ13409 (FLJ13409), mRNA. /FEA = mRNA |
| | | /GEN = FLJ13409 /PROD = hypothetical protein FLJ13409 /DB_XREF = gi: 13375835 /UG = Hs.30732 hypothetical |
| | | protein FLJ13409; KIAA1711 protein /FL = gb: NM_024617.1 |
| 315 | 221014_s_at | gb: NM_031296.1 /DEF = Homo sapiens hypothetical protein DKFZp434G099 (DKFZP434G099), mRNA. /FEA = mRNA |
| | | /GEN = DKFZP434G099 /PROD = hypothetical protein DKFZp434G099 /DB_XREF = gi: 13786128 /FL = gb: |
| | | NM_031296.1 |
| 316 | 221428_s_at | gb: NM_030921.1 /DEF = Homo sapiens hypothetical protein DC42 (DC42), mRNA. /FEA = CDS /GEN = DC42 |
| | | /PROD = hypothetical protein DC42 /DB_XREF = gi: 13569880 /FL = gb: NM_030921.1 |
| 317 | 221580_s_at | gb: BC001972.1 /DEF = Homo sapiens, clone MGC: 5306, mRNA, complete cds. /FEA = mRNA /PROD = Unknown |
| | | (protein for MGC: 5306) /DB_XREF = gi: 12805036 /UG = Hs.301732 hypothetical protein MGC5306 /FL = gb: |
| | | BC001972.1 |
| 318 | 221613_s_at | gb: AL136598.1 /DEF = Homo sapiens mRNA; cDNA DKFZp564F2116 (from clone DKFZp564F2116); complete cds. |
| | | /FEA = mRNA /GEN = DKFZp564F2116 /PROD = hypothetical protein /DB_XREF = gi: 13276696 /UG = Hs.83954 |
| | | protein associated with PRK1 /FL = gb: AL136598.1 |
| 319 | 222103_at | Consensus includes gb: AI434345 /FEA = EST /DB_XREF = gi: 4295529 /DB_XREF = est: ti48b06.x1 |
| | | /CLONE = IMAGE: 2133683 /UG = Hs.36908 activating transcription factor 1 /FL = gb: NM_005171.1 |
| 320 | 222119_s_at | Consensus includes gb: AL117620.1 /DEF = Homo sapiens mRNA; cDNA DKFZp564K2364 (from clone |
| | | DKFZp564K2364); partial cds. /FEA = mRNA /GEN = DKFZp564K2364 /PROD = hypothetical protein |
| | | /DB_XREF = gi: 5912200 /UG = Hs.284289 vitiligo-associated protein VIT-1 |
| 321 | 222148_s_at | Consensus includes gb: BF688108 /FEA = EST /DB_XREF = gi: 11973516 /DB_XREF = est: 602067332F1 |
| | | /CLONE = IMAGE: 4066422 /UG = Hs.14202 hypothetical protein FLJ11040 |
| 322 | 222156_x_at | Consensus includes gb: AK022459.1 /DEF = Homo sapiens cDNA FLJ12397 fis, clone MAMMA1002769, weakly similar to |
| | | Homo sapiens cell cycle progression restoration 8 protein (CPR8) mRNA. /FEA = mRNA. /DB_XREF = gi: 10433861 |
| | | /UG = Hs.82506 KIAA1254 protein |

TABLE 1B

| HD | | |
|---|---|---|
| Bio- | | |
| marker | Amersham | |
| Number | Probe_Name | Amersham Description |
| 1 | NM_016587.1_PROBE1 | “HOMO SAPIENS CHROMOBOX HOMOLOG 3 (HP1 GAMMA HOMOLOG, DROSOPHILA) (CBX3), |
| | | MRNA.” |
| 2 | NM_014267.1_PROBE1 | “HOMO SAPIENS SMALL ACIDIC PROTEIN (IMAGE145052), MRNA.” |
| 3 | NM_006265.1_PROBE1 | “HOMO SAPIENS RAD21 HOMOLOG (S. POMBE) (RAD21), MRNA.” |
| 4 | NM_018834.1_PROBE1 | “HOMO SAPIENS MATRIN 3 (MATR3), MRNA.” |
| 5 | 2479227CB1_PROBE1 | “HOMO SAPIENS UBIQUITIN-CONJUGATING ENZYME E2D 3 (UBC4/5 HOMOLOG, YEAST) (UBE2D3), |
| | | MRNA” |
| 6 | 011050CB1_PROBE1 | “HOMO SAPIENS LPS-INDUCED TNF-ALPHA FACTOR (PIG7), MRNA” |
| 7 | NM_002710.1_PROBE1 | “HOMO SAPIENS PROTEIN PHOSPHATASE 1, CATALYTIC SUBUNIT, GAMMA ISOFORM (PPP1CC), |
| | | MRNA.” |
| 8 | NM_006407.2_PROBE1 | “HOMO SAPIENS VITAMIN A RESPONSIVE; CYTOSKELETON RELATED (JWA), MRNA.” |
| 9 | NM_014670.1_PROBE1 | “HOMO SAPIENS BASIC LEUCINE-ZIPPER PROTEIN BZAP45 (BZAP45), MRNA.” |
| 10 | NM_013995.1_PROBE1 | “HOMO SAPIENS LYSOSOMAL-ASSOCIATED MEMBRANE PROTEIN 2 (LAMP2), TRANSCRIPT |
| | | VARIANT LAMP2B, MRNA.” |
| 11 | 151430.1_PROBE1 | “HOMO SAPIENS RAP1B, MEMBER OF RAS ONCOGENE FAMILY (RAP1B), MRNA” |
| 12 | NM_016127.1_PROBE1 | “HOMO SAPIENS HYPOTHETICAL PROTEIN MGC8721 (MGC8721), MRNA” |
| 13 | NM_003144.2_PROBE1 | “HOMO SAPIENS SIGNAL SEQUENCE RECEPTOR, ALPHA (TRANSLOCON-ASSOCIATED PROTEIN |
| | | ALPHA) (SSR1), MRNA.” |
| 14 | NM_012215.1_PROBE1 | “HOMO SAPIENS MENINGIOMA EXPRESSED ANTIGEN 5 (HYALURONIDASE) (MGEA5), MRNA.” |
| 15 | NM_004261.1_PROBE1 | “HOMO SAPIENS 15 KDA SELENOPROTEIN (SEP15), MRNA.” |
| 16 | NM_001967.2_PROBE1 | “HOMO SAPIENS EUKARYOTIC TRANSLATION INITIATION FACTOR 4A, ISOFORM 2 (EIF4A2), |
| | | MRNA” |
| 17 | NM_004986.1_PROBE1 | “HOMO SAPIENS KINECTIN 1 (KINESIN RECEPTOR) (KTN1), MRNA.” |
| 18 | 1330593CB1_PROBE1 | “HOMO SAPIENS DEK ONCOGENE (DNA BINDING) (DEK), MRNA” |
| 19 | AL136807_PROBE1 | HOMO SAPIENS MRNA; CDNA DKFZP434L1621 (FROM CLONE DKFZP434L1621); COMPLETE CDS |
| 20 | NM_001530.1_PROBE1 | “HOMO SAPIENS HYPOXIA-INDUCIBLE FACTOR 1, ALPHA SUBUNIT (BASIC HELIX-LOOP-HELIX |
| | | TRANSCRIPTION FACTOR) (HIF1A), MRNA.” |
| 21 | NM_000700.1_PROBE1 | “HOMO SAPIENS ANNEXIN A1 (ANXA1), MRNA.” |
| 22 | NM_005642.2_PROBE1 | “HOMO SAPIENS TAF7 RNA POLYMERASE II, TATA BOX BINDING PROTEIN (TBP)-ASSOCIATED |
| | | FACTOR, 55 KD (TAF7), MRNA.” |
| 23 | NM_012433.1_PROBE1 | “HOMO SAPIENS SPLICING FACTOR 3B, SUBUNIT 1, 155 KD (SF3B1), MRNA” |
| 24 | NM_014739.1_PROBE1 | “HOMO SAPIENS KIAA0164 GENE PRODUCT (KIAA0164), MRNA.” |
| 25 | NM_006276.2_PROBE1 | “HOMO SAPIENS SPLICING FACTOR, ARGININE/SERINE-RICH 7 (35 KD) (SFRS7), MRNA.” |
| 26 | NM_014819.1_PROBE1 | “HOMO SAPIENS KIAA0438 GENE PRODUCT (KIAA0438), MRNA” |
| 27 | NM_006164.1_PROBE1 | “HOMO SAPIENS NUCLEAR FACTOR (ERYTHROID-DERIVED 2)-LIKE 2 (NFE2L2), MRNA” |
| 28 | 2101110CB1_PROBE1 | “HOMO SAPIENS SUMO-1 ACTIVATING ENZYME SUBUNIT 2 (UBA2), MRNA” |
| 29 | NM_003851.1_PROBE1 | “HOMO SAPIENS CELLULAR REPRESSOR OF E1A-STIMULATED GENES (CREG), MRNA.” |
| 30 | NM_001356.2_PROBE1 | “HOMO SAPIENS DEAD/H (ASP-GLU-ALA-ASP/HIS) BOX POLYPEPTIDE 3 (DDX3), TRANSCRIPT |
| | | VARIANT 2, MRNA.” |
| 31 | 2903615CB1_PROBE1 | “HUMAN CAPPING PROTEIN ALPHA MRNA, PARTIAL CDS” |
| 32 | U03851_PROBE1 | “HUMAN CAPPING PROTEIN ALPHA MRNA, PARTIAL CDS” |
| 33 | 1302117CB1_PROBE1 | “HOMO SAPIENS RIBOSOMAL PROTEIN S3A (RPS3A), MRNA” |
| 34 | 898944.4_PROBE1 | “HOMO SAPIENS CHROMOSOME 2 OPEN READING FRAME 6 (C2ORF6), MRNA” |
| 35 | 1489160CB1_PROBE1 | “HOMO SAPIENS SH3 DOMAIN BINDING GLUTAMIC ACID-RICH PROTEIN LIKE (SH3BGRL), MRNA” |
| 36 | NM_002709.1_PROBE1 | “HOMO SAPIENS PROTEIN PHOSPHATASE 1, CATALYTIC SUBUNIT, BETA ISOFORM (PPP1CB), |
| | | MRNA.” |
| 37 | NM_022037.1_PROBE1 | “HOMO SAPIENS TIA1 CYTOTOXIC GRANULE-ASSOCIATED RNA BINDING PROTEIN (TIA1), |
| | | TRANSCRIPT VARIANT 1, MRNA” |
| 38 | NM_004725.1_PROBE1 | “HOMO SAPIENS BUB3 BUDDING UNINHIBITED BY BENZIMIDAZOLES 3 HOMOLOG (YEAST) |
| | | (BUB3), MRNA.” |
| 39 | NM_003372.2_PROBE1 | “HOMO SAPIENS VON HIPPEL-LINDAU BINDING PROTEIN 1 (VBP1), MRNA.” |
| 40 | NM_001814.1_PROBE1 | “HOMO SAPIENS CATHEPSIN C (CTSC), MRNA.” |
| 41 | NM_015317.1_PROBE1 | “HOMO SAPIENS PUMILIO HOMOLOG 2 (DROSOPHILA) (PUM2), MRNA.” |
| 42 | 1236363CB1_PROBE1 | “HOMO SAPIENS UBIQUITIN-CONJUGATING ENZYME E2N (UBC13 HOMOLOG, YEAST) (UBE2N), |
| | | MRNA” |
| 43 | NM_007106.1_PROBE1 | “HOMO SAPIENS UBIQUITIN-LIKE 3 (UBL3), MRNA.” |
| 44 | NM_014402.1_PROBE1 | “HOMO SAPIENS LOW MOLECULAR MASS UBIQUINONE-BINDING PROTEIN (9.5 KD) (QP-C), |
| | | MRNA.” |
| 45 | NM_018471.1_PROBE1 | “HOMO SAPIENS UNCHARACTERIZED HYPOTHALAMUS PROTEIN HT010 (HT010), MRNA.” |
| 46 | NM_002480.1_PROBE1 | “HOMO SAPIENS PROTEIN PHOSPHATASE 1, REGULATORY (INHIBITOR) SUBUNIT 12A (PPP1R12A), |
| | | MRNA.” |
| 47 | NM_002806.1_PROBE1 | “HOMO SAPIENS PROTEASOME (PROSOME, MACROPAIN) 26S SUBUNIT, ATPASE, 6 (PSMC6), |
| | | MRNA.” |
| 48 | 2581158CB1_PROBE1 | “HOMO SAPIENS METHYLENE TETRAHYDROFOLATE DEHYDROGENASE (NAD+ DEPENDENT), |
| | | METHENYLTETRAHYDROFOLATE CYCLOHYDROLASE (MTHFD2), NUCLEAR GENE ENCODING |
| | | MITOCHONDRIAL PROTEIN, MRNA” |
| 49 | NM_007282.1_PROBE1 | “HOMO SAPIENS RING FINGER PROTEIN 13 (RNF13), MRNA.” |
| 50 | NM_004896.1_PROBE1 | “HOMO SAPIENS VACUOLAR PROTEIN SORTING 26 (YEAST) (VPS26), MRNA.” |
| 51 | NM_016107.1_PROBE1 | “HOMO SAPIENS ZINC FINGER RNA BINDING PROTEIN (ZFR), MRNA” |
| 52 | 2659255CB1_PROBE1 | “HOMO SAPIENS PROTEOGLYCAN 1, SECRETORY GRANULE (PRG1), MRNA” |
| 53 | Y10183_PROBE1 | H. SAPIENS MRNA FOR MEMD PROTEIN |
| 54 | NM_002835.1_PROBE1 | “HOMO SAPIENS PROTEIN TYROSINE PHOSPHATASE, NON-RECEPTOR TYPE 12 (PTPN12), MRNA.” |
| 55 | NM_006055.1_PROBE1 | “HOMO SAPIENS LANC (BACTERIAL LANTIBIOTIC SYNTHETASE COMPONENT C)-LIKE 1 (LANCL1), |
| | | MRNA.” |
| 56 | NM_014781.1_PROBE1 | “HOMO SAPIENS LIKELY ORTHOLOG OF MOUSE COILED COIL FORMING PROTEIN 1 (KIAA0203), |
| | | MRNA.” |
| 57 | NM_001166.2_PROBE1 | “HOMO SAPIENS BACULOVIRAL IAP REPEAT-CONTAINING 2 (BIRC2), MRNA.” |
| 58 | NM_003100.1_PROBE1 | “HOMO SAPIENS SORTING NEXIN 2 (SNX2), MRNA” |
| 59 | NM_006710.1_PROBE1 | “HOMO SAPIENS COP9 HOMOLOG (COP9), MRNA.” |
| 60 | NM_001550.1_PROBE1 | “HOMO SAPIENS INTERFERON-RELATED DEVELOPMENTAL REGULATOR 1 (IFRD1), MRNA” |
| 61 | NM_004779.1_PROBE1 | “HOMO SAPIENS CCR4-NOT TRANSCRIPTION COMPLEX, SUBUNIT 8 (CNOT8), MRNA.” |
| 62 | BC007655_PROBE1 | “HOMO SAPIENS, PROTEIN PHOSPHATASE 1, REGULATORY (INHIBITOR) SUBUNIT 2, CLONE |
| | | MGC: 1327 IMAGE: 3346573, MRNA, COMPLETE CDS” |
| 63 | NM_003187.1_PROBE1 | “HOMO SAPIENS TATA BOX BINDING PROTEIN (TBP)-ASSOCIATED FACTOR, RNA |
| | | POLYMERASE II, G, 32 KD (TAF2G), MRNA.” |
| 64 | NM_015423.1_PROBE1 | “HOMO SAPIENS AMINOADIPATE-SEMIALDEHYDE DEHYDROGENASE-PHOSPHOPANTETHEINYL |
| | | TRANSFERASE (AASDHPPT), MRNA.” |
| 65 | NM_024536.1_PROBE1 | “HOMO SAPIENS HYPOTHETICAL PROTEIN FLJ22678 (FLJ22678), MRNA” |
| 66 | AL080084_PROBE1 | “HOMO SAPIENS, CGI-100 PROTEIN, CLONE MGC: 5366 IMAGE: 3048959, MRNA, COMPLETE CDS” |
| 67 | NM_003588.1_PROBE1 | “HOMO SAPIENS CULLIN 4B (CUL4B), MRNA.” |
| 68 | NM_012428.1_PROBE2 | “HOMO SAPIENS STROMAL CELL DERIVED FACTOR RECEPTOR 1 (SDFR1), TRANSCRIPT VARIANT |
| | | BETA, MRNA” |
| 69 | 1452741.7_PROBE1 | “HOMO SAPIENS HYPOTHETICAL PROTEIN FROM BCRA2 REGION (CG005), MRNA” |
| 70 | NM_016614.1_PROBE1 | “HOMO SAPIENS TRAF AND TNF RECEPTOR-ASSOCIATED PROTEIN (AD022), MRNA” |
| 71 | NM_015571.1_PROBE1 | “HOMO SAPIENS SUMO-1-SPECIFIC PROTEASE (SUSP1), MRNA” |
| 72 | NM_002816.1_PROBE1 | “HOMO SAPIENS PROTEASOME (PROSOME, MACROPAIN) 26S SUBUNIT, NON-ATPASE, 12 |
| | | (PSMD12), MRNA.” |
| 73 | NM_022845.1_PROBE1 | “HOMO SAPIENS CORE-BINDING FACTOR, BETA SUBUNIT (CBFB), TRANSCRIPT VARIANT 1, |
| | | MRNA.” |
| 74 | NM_003816.1_PROBE1 | “HOMO SAPIENS A DISINTEGRIN AND METALLOPROTEINASE DOMAIN 9 (MELTRIN GAMMA) |
| | | (ADAM9), MRNA.” |
| 75 | NM_022977.1_PROBE1 | “HOMO SAPIENS FATTY-ACID-COENZYME A LIGASE, LONG-CHAIN 4 (FACL4), TRANSCRIPT |
| | | VARIANT 2, MRNA.” |
| 76 | AL353950_PROBE1 | HOMO SAPIENS MRNA; CDNA DKFZP761L0516 (FROM CLONE DKFZP761L0516); COMPLETE CDS |
| 77 | NM_021132.1_PROBE1 | “HOMO SAPIENS PROTEIN PHOSPHATASE 3 (FORMERLY 2B), CATALYTIC SUBUNIT, |
| | | BETA ISOFORM (CALCINEURIN A BETA) (PPP3CB), MRNA.” |
| 78 | NM_000104.2_PROBE1 | “HOMO SAPIENS CYTOCHROME P450, SUBFAMILY I (DIOXIN-INDUCIBLE), POLYPEPTIDE 1 |
| | | (GLAUCOMA 3, PRIMARY INFANTILE) (CYP1B1), MRNA.” |
| 79 | NM_004236.1_PROBE1 | “HOMO SAPIENS THYROID RECEPTOR INTERACTING PROTEIN 15 (TRIP15), MRNA.” |
| 80 | NM_006751.1_PROBE1 | “HOMO SAPIENS SPERM SPECIFIC ANTIGEN 2 (SSFA2), MRNA.” |
| 81 | NM_014043.1_PROBE1 | “HOMO SAPIENS DKFZP564O123 PROTEIN (DKFZP564O123), MRNA.” |
| 82 | NM_004124.1_PROBE1 | “HOMO SAPIENS GLIA MATURATION FACTOR, BETA (GMFB), MRNA.” |
| 83 | NM_015344.1_PROBE1 | “HOMO SAPIENS LEPTIN RECEPTOR OVERLAPPING TRANSCRIPT-LIKE 1 (LEPROTL1), MRNA.” |
| 84 | NM_012290.1_PROBE1 | “HOMO SAPIENS TOUSLED-LIKE KINASE 1 (TLK1), MRNA.” |
| 85 | NM_014873.1_PROBE1 | “HOMO SAPIENS KIAA0205 GENE PRODUCT (KIAA0205), MRNA.” |
| 86 | NM_022826.1_PROBE1 | “HOMO SAPIENS AXOTROPHIN (AXOT), MRNA” |
| 87 | NM_022826.1_PROBE1 | “HOMO SAPIENS AXOTROPHIN (AXOT), MRNA” |
| 88 | NM_003859.1_PROBE1 | “HOMO SAPIENS DOLICHYL-PHOSPHATE MANNOSYLTRANSFERASE POLYPEPTIDE 1, CATALYTIC |
| | | SUBUNIT (DPM1), MRNA.” |
| 89 | NM_003810.1_PROBE1 | “HOMO SAPIENS TUMOR NECROSIS FACTOR (LIGAND) SUPERFAMILY, MEMBER 10 (TNFSF10), |
| | | MRNA.” |
| 90 | NM_032991.1_PROBE1 | “HOMO SAPIENS CASPASE 3, APOPTOSIS-RELATED CYSTEINE PROTEASE (CASP3), TRANSCRIPT |
| | | VARIANT BETA, MRNA.” |
| 91 | NM_004354.1_PROBE1 | “HOMO SAPIENS CYCLIN G2 (CCNG2), MRNA.” |
| 92 | NM_007373.1_PROBE1 | “HOMO SAPIENS SOC-2 SUPPRESSOR OF CLEAR HOMOLOG (C. ELEGANS) (SHOC2), MRNA.” |
| 93 | NM_003453.1_PROBE1 | “HOMO SAPIENS ZINC FINGER PROTEIN 198 (ZNF198), MRNA.” |
| 94 | NM_014016.1_PROBE1 | “HOMO SAPIENS SAC1 SUPPRESSOR OF ACTIN MUTATIONS 1-LIKE (YEAST) (SACMIL), MRNA.” |
| 95 | NM_006323.1_PROBE1 | “HOMO SAPIENS SEC24 RELATED GENE FAMILY, MEMBER B (S. CEREVISIAE) (SEC24B), MRNA.” |
| 96 | NM_005638.1_PROBE1 | “HOMO SAPIENS SYNAPTOBREVIN-LIKE 1 (SYBL1), MRNA.” |
| 97 | NM_004079.1_PROBE1 | “HOMO SAPIENS CATHEPSIN S (CTSS), MRNA.” |
| 98 | NM_002485.2_PROBE1 | “HOMO SAPIENS NIJMEGEN BREAKAGE SYNDROME 1 (NIBRIN) (NBS1), MRNA.” |
| 99 | NM_015387.1_PROBE1 | “HOMO SAPIENS PREIMPLANTATION PROTEIN 3 (PREI3), MRNA.” |
| 100 | NM_005783.1_PROBE1 | “HOMO SAPIENS ATP BINDING PROTEIN ASSOCIATED WITH CELL DIFFERENTIATION (APACD), |
| | | MRNA.” |
| 101 | NM_014857.1_PROBE1 | “HOMO SAPIENS KIAA0471 GENE PRODUCT (KIAA0471), MRNA.” |
| 102 | NM_020199.1_PROBE1 | “HOMO SAPIENS HTGN29 PROTEIN (HTGN29), MRNA.” |
| 103 | NM_014639.1_PROBE1 | “HOMO SAPIENS KIAA0372 GENE PRODUCT (KIAA0372), MRNA.” |
| 104 | NM_013450.1_PROBE1 | “HOMO SAPIENS BROMODOMAIN ADJACENT TO ZINC FINGER DOMAIN, 2B (BAZ2B), MRNA.” |
| 105 | NM_000321.1_PROBE1 | “HOMO SAPIENS RETINOBLASTOMA 1 (INCLUDING OSTEOSARCOMA) (RB1), MRNA.” |
| 106 | NM_016248.1_PROBE1 | “HOMO SAPIENS A KINASE (PRKA) ANCHOR PROTEIN 11 (AKAP11), TRANSCRIPT VARIANT 1, |
| | | MRNA” |
| 107 | 1093761.2_PROBE1 | “HOMO SAPIENS HYPOTHETICAL PROTEIN FLJ11149 (FLJ11149), MRNA” |
| 108 | NM_015216.1_PROBE1 | “HOMO SAPIENS KIAA0433 PROTEIN (KIAA0433), MRNA.” |
| 109 | NM_003916.1_PROBE1 | “HOMO SAPIENS ADAPTOR-RELATED PROTEIN COMPLEX 1, SIGMA 2 SUBUNIT (AP1S2), MRNA.” |
| 110 | NM_003916.1_PROBE1 | “HOMO SAPIENS ADAPTOR-RELATED PROTEIN COMPLEX 1, SIGMA 2 SUBUNIT (AP1S2), MRNA.” |
| 111 | NM_000788.1_PROBE1 | “HOMO SAPIENS DEOXYCYTIDINE KINASE (DCK), MRNA.” |
| 112 | NM_006416.1_PROBE1 | “HOMO SAPIENS SOLUTE CARRIER FAMILY 35 (CMP-SIALIC ACID TRANSPORTER), MEMBER 1 |
| | | (SLC35A1), MRNA.” |
| 113 | NM_014832.1_PROBE1 | “HOMO SAPIENS KIAA0603 GENE PRODUCT (KIAA0603), MRNA.” |
| 114 | NM_014832.1_PROBE1 | “HOMO SAPIENS KIAA0603 GENE PRODUCT (KIAA0603), MRNA.” |
| 115 | NM_002789.1_PROBE1 | “HOMO SAPIENS PROTEASOME (PROSOME, MACROPAIN) SUBUNIT, ALPHA TYPE, 4 (PSMA4), |
| | | MRNA.” |
| 116 | NM_004482.2_PROBE1 | “HOMO SAPIENS UPD-N-ACETYL-ALPHA-D-GALACTOSAMINE: POLYPEPTIDE N- |
| | | ACETYLGLACTOSAMINYLTRANSFERASE 3 (GALNAC-T3) (GALNT3), MRNA.” |
| 117 | NM_005977.1_PROBE1 | “HOMO SAPIENS RING FINGER PROTEIN (C3H2C3 TYPE) 6 (RNF6), MRNA.” |
| 118 | NM_016255.1_PROBE1 | “HOMO SAPIENS FAMILY WITH SEQUENCE SIMILARITY 8, MEMBER A1 (FAM8A1), MRNA.” |
| 119 | 2518753CB1_PROBE1 | “HOMO SAPIENS DKFZP547E2110 PROTEIN (DKFZP547E2110), MRNA” |
| 120 | NM_002970.1_PROBE1 | “HOMO SAPIENS SPERMIDINE/SPERMINE N1-ACETYLTRANSFERASE (SAT), MRNA.” |
| 121 | NM_014679.1_PROBE1 | “HOMO SAPIENS KIAA0092 GENE PRODUCT (KIAA0092), MRNA.” |
| 122 | 1382919.97_PROBE1 | “HOMO SAPIENS MUSCLEBLIND-LIKE PROTEIN MBLL39 (MBLL39) TRANSCRIPT VARIANT 1, |
| | | MRNA” |
| 123 | NM_006526.1_PROBE1 | “HOMO SAPIENS ZINC FINGER PROTEIN 217 (ZNF217), MRNA.” |
| 124 | NM_012198.1_PROBE1 | “HOMO SAPIENS GRANCALCIN, EF-HAND CALCIUM BINDING PROTEIN (GCA), MRNA.” |
| 125 | NM_014880.1_PROBE1 | “HOMO SAPIENS KIAA0022 GENE PRODUCT (KIAA0022), MRNA.” |
| 126 | NM_004688.1_PROBE1 | “HOMO SAPIENS N-MYC (AND STAT) INTERACTOR (NMI), MRNA.” |
| 127 | NM_005999.1_PROBE1 | “HOMO SAPIENS TRANSLIN-ASSOCIATED FACTOR X (TSNAX), MRNA.” |
| 128 | NM_006895.1_PROBE1 | “HOMO SAPIENS HISTAMINE N-METHYLTRANSFERASE (HNMT), MRNA.” |
| 129 | NM_000097.1_PROBE1 | “HOMO SAPIENS COPROPORPHYRINOGEN OXIDASE (COPROPORPHYRIA, HARDEROPORPHYRIA) |
| | | (CPO), MRNA.” |
| 130 | NM_005038.1_PROBE1 | “HOMO SAPIENS PEPTIDYLPROLYL ISOMERASE D (CYCLOPHILIN D) (PPID), MRNA” |
| 131 | NM_001186.1_PROBE1 | “HOMO SAPIENS BTB AND CNC HOMOLOGY 1, BASIC LEUCINE ZIPPER TRANSCRIPTION FACTOR 1 |
| | | (BACH1), MRNA.” |
| 132 | 011189CB1_PROBE1 | “HOMO SAPIENS GTP CYCLOHYDROLASE 1 (DOPA-RESPONSIVE DYSTONIA) (GCH1), MRNA” |
| 133 | NM_001270.1_PROBE1 | “HOMO SAPIENS CHROMODOMAIN HELICASE DNA BINDING PROTEIN 1 (CHD1), MRNA.” |
| 134 | NM_006218.1_PROBE1 | “HOMO SAPIENS PHOSPHOINOSITIDE-3-KINASE, CATALYTIC, ALPHA POLYPEPTIDE (PIK3CA), |
| | | MRNA.” |
| 135 | NM_000153.1_PROBE1 | “HOMO SAPIENS GALACTOSYLCERAMIDASE (KRABBE DISEASE) (GALC), MRNA.” |
| 136 | NM_004508.1_PROBE1 | “HOMO SAPIENS ISOPENTENYL-DIPHOSPHATE DELTA ISOMERASE (IDI1), MRNA.” |
| 137 | NM_000110.2_PROBE1 | “HOMO SAPIENS DIHYDROPYRIMIDINE DEHYDROGENASE (DPYD), MRNA.” |
| 138 | NM_000963.1_PROBE1 | “HOMO SAPIENS PROSTAGLANDIN-ENDOPEROXIDE SYNTHASE 2 (PROSTAGLANDIN G/H |
| | | SYNTHASE AND CYCLOOXYGENASE) (PTGS2), MRNA.” |
| 139 | NM_001268.1_PROBE1 | “HOMO SAPIENS CHROMOSOME CONDENSATION 1-LIKE (CHC1L), MRNA.” |
| 140 | NM_000043.1_PROBE1 | “HOMO SAPIENS TUMOR NECROSIS FACTOR RECEPTOR SUPERFAMILY, MEMBER 6 (TNFRSF6), |
| | | MRNA.” |
| 141 | NM_002892.2_PROBE1 | “HOMO SAPIENS RETINOBLASTOMA BINDING PROTEIN 1 (RBBP1), TRANSCRIPT VARIANT 1, |
| | | MRNA” |
| 142 | NM_002907.2_PROBE1 | “HOMO SAPIENS RECQ PROTEIN-LIKE (DNA HELICASE Q1-LIKE) (RECQL), TRANSCRIPT |
| | | VARIANT 1, MRNA” |
| 143 | NM_001779.1_PROBE1 | “HOMO SAPIENS CD58 ANTIGEN, (LYMPHOCYTE FUNCTION-ASSOCIATED ANTIGEN 3) (CD58), |
| | | MRNA.” |
| 144 | NM_006915.1_PROBE1 | “HOMO SAPIENS RETINITIS PIGMENTOSA 2 (X-LINKED RECESSIVE) (RP2), MRNA.” |
| 145 | NM_004226.1_PROBE1 | “HOMO SAPIENS SERINE/THREONINE KINASE 17B (APOPTOSIS-INDUCING) (STK17B), MRNA.” |
| 146 | 1358031CB1_PROBE1 | “HOMO SAPIENS JANUS KINASE 2 (A PROTEIN TYROSINE KINASE) (JAK2), MRNA” |
| 147 | NM_014645.1_PROBE1 | “HOMO SAPIENS KIAA0635 GENE PRODUCT (KIAA0635), MRNA” |
| 148 | NM_015967.1_PROBE1 | “HOMO SAPIENS PROTEIN TYROSINE PHOSPHATASE, NON-RECEPTOR TYPE 22 (LYMPHOID) |
| | | (PTPN22), TRANSCRIPT VARIANT 1, MRNA.” |
| 149 | NM_021993.1_PROBE1 | “HOMO SAPIENS FUS INTERACTING PROTEIN (SERINE-ARGININE RICH) 2 (FUSIP2), MRNA.” |
| 150 | NM_003418.1_PROBE1 | “HOMO SAPIENS ZINC FINGER PROTEIN 9 (A CELLULAR RETROVIRAL NUCLEIC ACID BINDING |
| | | PROTEIN) (ZNF9), MRNA” |
| 151 | NM_000072.1_PROBE1 | “HOMO SAPIENS CD36 ANTIGEN (COLLAGEN TYPE I RECEPTOR, THROMBOSPONDIN RECEPTOR) |
| | | (CD36), MRNA.” |
| 152 | NM_015364.1_PROBE1 | “HOMO SAPIENS MD-2 PROTEIN (MD-2), MRNA.” |
| 153 | 7478318CB1_PROBE1 | “HOMO SAPIENS MITOGEN-ACTIVATED PROTEIN KINASE KINASE KINASE 7 (MAP3K7), |
| | | TRANSCRIPT VARIANT A, MRNA” |
| 154 | NM_000167.1_PROBE1 | “HOMO SAPIENS GLYCEROL KINASE (GK), MRNA.” |
| 155 | NM_015874.1_PROBE1 | “HOMO SAPIENS H-2K BINDING FACTOR-2 (LOC51580), MRNA.” |
| 156 | NM_004902.1_PROBE1 | “HOMO SAPIENS RNA-BINDING REGION (RNP1, RRM) CONTAINING 2 (RNPC2), MRNA.” |
| 157 | NM_015032.1_PROBE1 | “HOMO SAPIENS KIAA0979 PROTEIN (KIAA0979), MRNA.” |
| 158 | NM_014350.1_PROBE1 | “HOMO SAPIENS TNF-INDUCED PROTEIN (GG2-1), MRNA.” |
| 159 | 014843CB1_PROBE1 | “HOMO SAPIENS RNA BINDING MOTIF PROTEIN 3 (RBM3), MRNA” |
| 160 | NM_006135.1_PROBE1 | “HOMO SAPIENS CAPPING PROTEIN (ACTIN FILAMENT) MUSCLE Z-LINE, ALPHA 1 (CAPZA1), |
| | | MRNA.” |
| 161 | 1100752.1_PROBE1 | “HOMO SAPIENS STUD PROTEIN MRNA, COMPLETE CDS” |
| 162 | NM_006016.1_PROBE1 | “HOMO SAPIENS CD164 ANTIGEN, SIALOMUCIN (CD164), MRNA.” |
| 163 | NM_014335.1_PROBE1 | “HOMO SAPIENS CREBBP/EP300 INHIBITORY PROTEIN 1 (CRI1), MRNA” |
| 164 | 2135776CB1_PROBE1 | “HOMO SAPIENS, KIAA1253 PROTEIN, CLONE MGC: 33018 IMAGE: 4831122, MRNA, COMPLETE |
| | | CDS” |
| 165 | 3767486CB1_PROBE1 | “HOMO SAPIENS SPLICING FACTOR, ARGININE/SERINE-RICH 3 (SFRS3), MRNA” |
| 166 | 1330056CB1_PROBE1 | “HOMO SAPIENS EUKARYOTIC TRANSLATION INITIATION FACTOR 3, SUBUNIT 6 (48 KD) (EIF3S6), |
| | | MRNA” |
| 167 | 198777.32_PROBE2 | “HOMO SAPIENS EUKARYOTIC TRANSLATION INITIATION FACTOR 5 (EIF5), MRNA” |
| 168 | 198777.32_PROBE1 | “HOMO SAPIENS EUKARYOTIC TRANSLATION INITIATION FACTOR 5 (EIF5), MRNA” |
| 169 | NM_004888.1_PROBE1 | “HOMO SAPIENS ATPASE, H+ TRANSPORTING, LYSOSOMAL (VACUOLAR PROTON PUMP), |
| | | MEMBER J (ATP6J), MRNA.” |
| 170 | NM_003400.2_PROBE1 | “HOMO SAPIENS EXPORTIN 1 (CRM1 HOMOLOG, YEAST) (XPO1), MRNA.” |
| 171 | NM_007208.1_PROBE1 | “HOMO SAPIENS MITOCHONDRIAL RIBOSOMAL PROTEIN L3 (MRPL3), MRNA.” |
| 172 | NM_004060.2_PROBE1 | “HOMO SAPIENS CYCLIN G1 (CCNG1), MRNA.” |
| 173 | NM_002129.2_PROBE1 | “HOMO SAPIENS HIGH-MOBILITY GROUP (NONHISTONE CHROMOSOMAL) PROTEIN 2 (HMG2), |
| | | MRNA.” |
| 174 | AB014560_PROBE1 | “HOMO SAPIENS MRNA FOR KIAA0660 PROTEIN, COMPLETE CDS” |
| 175 | NM_019895.1_PROBE1 | “HOMO SAPIENS CHROMOSOME 3 OPEN READING FRAME 4 (C3ORF4), MRNA.” |
| 176 | 1758973CB1_PROBE1 | “HOMO SAPIENS TRANSLOCATION PROTEIN 1 (TLOC1), MRNA” |
| 177 | NM_033535_PROBE1 | “HOMO SAPIENS F-BOX AND LEUCINE-RICH REPEAT PROTEIN 5 (FBXL5), TRANSCRIPT VARIANT 2, |
| | | MRNA.” |
| 178 | AF006516_PROBE1 | “HOMO SAPIENS EPS8 BINDING PROTEIN E3B1 MRNA, COMPLETE CDS” |
| 179 | NM_005470.1_PROBE1 | “HOMO SAPIENS SPECTRIN SH3 DOMAIN BINDING PROTEIN 1 (SSH3BP1), MRNA” |
| 180 | D86550_PROBE1 | “HUMAN MRNA FOR SERINE/THREONINE PROTEIN KINASE, COMPLETE CDS” |
| 181 | NM_003968.1_PROBE1 | “HOMO SAPIENS UBIQUITIN-ACTIVATING ENZYME E1C (UBA3 HOMOLOG, YEAST) (UBE1C), |
| | | MRNA.” |
| 182 | NM_004892.2_PROBE1 | “HOMO SAPIENS SEC22 VESICLE TRAFFICKING PROTEIN-LIKE 1 (S. CEREVISIAE) (SEC22L1), |
| | | MRNA.” |
| 183 | NM_003605.2_PROBE2 | “HOMO SAPIENS O-LINKED N-ACETYLGLUCOSAMINE (GLCNAC) TRANSFERASE (UDP-N- |
| | | ACETYLGLUCOSAMINE: POLYPEPTIDE-N-ACETYLGLUCOSAMINYL TRANSFERASE) (OGT), |
| | | MRNA.” |
| 184 | NM_005445.1_PROBE1 | “HOMO SAPIENS CHONDROITIN SULFATE PROTEOGLYCAN 6 (BAMACAN) (CSPG6), MRNA.” |
| 185 | NM_015224.1_PROBE1 | “HOMO SAPIENS KIAA1105 PROTEIN (RAP140), MRNA.” |
| 186 | NM_015509.1_PROBE1 | “HOMO SAPIENS DKFZP566B183 PROTEIN (DKFZP566B183), MRNA.” |
| 187 | NM_004330.1_PROBE1 | “HOMO SAPIENS BCL2/ADENOVIRUS E1B 19 KD INTERACTING PROTEIN 2 (BNIP2), MRNA.” |
| 188 | AF081567_PROBE1 | “HOMO SAPIENS DEATH ASSOCIATED PROTEIN 4 (DAP4) MRNA, COMPLETE CDS” |
| 189 | NM_021144.1_PROBE1 | “HOMO SAPIENS PC4 AND SFRS1 INTERACTING PROTEIN 1 (PSIP1), MRNA” |
| 190 | AF055376_PROBE1 | “HOMO SAPIENS SHORT FORM TRANSCRIPTION FACTOR C-MAF (C-MAF) MRNA, COMPLETE CDS” |
| 191 | 253580.1_PROBE1 | “HOMO SAPIENS CDNA FLJ90481 FIS, CLONE NT2RP3002900” |
| 192 | NM_004180.1_PROBE1 | “HOMO SAPIENS TRAF FAMILY MEMBER-ASSOCIATED NFKB ACTIVATOR (TANK), MRNA.” |
| 193 | NM_030755.1_PROBE1 | “HOMO SAPIENS THIOREDOXIN DOMAIN-CONTAINING (TXNDC), MRNA.” |
| 194 | NM_007186.1_PROBE1 | “HOMO SAPIENS CENTROSOMAL PROTEIN 2 (CEP2), MRNA.” |
| 195 | NM_032303.1_PROBE1 | “HOMO SAPIENS HYPOTHETICAL PROTEIN MGC10940 (MGC10940), MRNA.” |
| 196 | NM_003797.1_PROBE1 | “HOMO SAPIENS EMBRYONIC ECTODERM DEVELOPMENT (EED), MRNA” |
| 197 | AB029006_PROBE1 | “HOMO SAPIENS MRNA FOR KIAA1083 PROTEIN, COMPLETE CDS” |
| 198 | NM_015864.2_PROBE1 | “HOMO SAPIENS CHROMOSOME 6 OPEN READING FRAME 32 (C6ORF32), MRNA” |
| 199 | NM_014350.1_PROBE1 | “HOMO SAPIENS TNF-INDUCED PROTEIN (GG2-1), MRNA.” |
| 200 | NM_000318.1_PROBE1 | “HOMO SAPIENS PEROXISOMAL MEMBRANE PROTEIN 3 (35 KD, ZELLWEGER SYNDROME) |
| | | (PXMP3), MRNA.” |
| 201 | NM_004071.1_PROBE1 | “HOMO SAPIENS CDC-LIKE KINASE1 (CLK1), MRNA.” |
| 202 | NM_001165.2_PROBE1 | “HOMO SAPIENS BACULOVIRAL IAP REPEAT-CONTAINING 3 (BIRC3), MRNA.” |
| 203 | NM_006313.1_PROBE1 | “HOMO SAPIENS UBIQUITIN SPECIFIC PROTEASE 15 (USP15), MRNA.” |
| 204 | NM_000314.1_PROBE1 | “HOMO SAPIENS PHOSPHATASE AND TENSIN HOMOLOG (MUTATED IN MULTIPLE ADVANCED |
| | | CANCERS 1) (PTEN), MRNA.” |
| 205 | 169384CB1_PROBE1 | “HOMO SAPIENS TUMOR PROTEIN, TRANSLATIONALLY-CONTROLLED 1 (TPT1), MRNA” |
| 206 | 5472755CB1_PROBE1 | HOMO SAPIENS MRNA; CDNA DKFZP667O2119 (FROM CLONE DKFZP667O2119) |
| 207 | D63477_PROBE1 | “HUMAN MRNA FOR KIAA0143 GENE, PARTIAL CDS” |
| 208 | 1453892.3_PROBE1 | HOMO SAPIENS MRNA; CDNA DKFZP564B0769 (FROM CLONE DKFZPS64B0769); PARTIAL CDS |
| 209 | AL080186_PROBE1 | HOMO SAPIENS MRNA; CDNA DKFZP564B0769 (FROM CLONE DKFZP564B0769); PARTIAL CDS |
| 210 | AL117407_PROBE1 | HOMO SAPIENS MRNA; CDNA DKFZP434D2050 (FROM CLONE DKFZP434D2050); PARTIAL CDS |
| 211 | NM_015355.1_PROBE1 | “HOMO SAPIENS JOINED TO JAZF1 (JJAZ1), MRNA” |
| 212 | NM_015208.1_PROBE1 | “HOMO SAPIENS KIAA0B74 PROTEIN (KIAA0874), MRNA.” |
| 213 | AB014527_PROBE1 | “HOMO SAPIENS MRNA FOR KIAA0627 PROTEIN, PARTIAL CDS” |
| 214 | NM_005611.1_PROBE1 | “HOMO SAPIENS RETINOBLASTOMA-LIKE 2 (P130) (RBL2), MRNA.” |
| 215 | AB011102_PROBE1 | “HOMO SAPIENS MRNA FOR KIAA0530 PROTEIN, PARTIAL CDS” |
| 216 | 2279216CB1_PROBE1 | “HOMO SAPIENS CDNA FLJ13142 FIS, CLONE NT2RP3003212, MODERATELY SIMILAR TO RATTUS |
| | | NORVEGICUS LAMINA ASSOCIATED POLYPEPTIDE 1C (LAP1C) MRNA” |
| 217 | 1177116.1_PROBE1 | “HOMO SAPIENS HYPOTHETICAL PROTEIN FLJ34588 (FLJ34588), MRNA” |
| 218 | M82882_PROBE1 | HUMAN CIS-ACTING SEQUENCE |
| 219 | NM_006330.1_PROBE1 | “HOMO SAPIENS LYSOPHOSPHOLIPASE I (LYPLA1), MRNA.” |
| 220 | D87445_PROBE1 | “HOMO SAPIENS MRNA FOR KIAA0256 PROTEIN, PARTIAL CDS” |
| 221 | D26069_PROBE1 | “HUMAN MRNA FOR KIAA0041 GENE, PARTIAL CDS” |
| 222 | AB023173_PROBE1 | “HOMO SAPIENS MRNA FOR KIAA0956 PROTEIN, PARTIAL CDS” |
| 223 | AB023182_PROBE1 | “HOMO SAPIENS MRNA FOR KIAA0965 PROTEIN, PARTIAL CDS” |
| 224 | AB014550_PROBE1 | “HOMO SAPIENS MRNA FOR KIAA0650 PROTEIN, PARTIAL CDS” |
| 225 | NM_002838.1_PROBE1 | “HOMO SAPIENS PROTEIN TYROSINE PHOSPHATASE, RECEPTOR TYPE, C (PTPRC), MRNA.” |
| 226 | AB018319_PROBE1 | “HOMO SAPIENS MRNA FOR KIAA0776 PROTEIN, PARTIAL CDS” |
| 227 | AB029032_PROBE1 | “HOMO SAPIENS MRNA FOR KIAA1109 PROTEIN, PARTIAL CDS” |
| 228 | AB020663_PROBE1 | “HOMO SAPIENS MRNA FOR KIAA0856 PROTEIN, PARTIAL CDS” |
| 229 | AB011542_PROBE1 | “HOMO SAPIENS MRNA FOR MEGF9, PARTIAL CDS” |
| 230 | 3087724CB1_PROBE1 | “HOMO SAPIENS SEC23 HOMOLOG A (S. CEREVISIAE) (SEC23A), MRNA” |
| 231 | AL080063_PROBE1 | HOMO SAPIENS MRNA; CDNA DKFZP564I052 (FROM CLONE DKFZP564I052) |
| 232 | AB020689_PROBE1 | “HOMO SAPIENS MRNA FOR KIAA0882 PROTEIN, PARTIAL CDS” |
| 233 | AB023163_PROBE1 | “HOMO SAPIENS MRNA FOR KIAA0946 PROTEIN, PARTIAL CDS” |
| 234 | D50926_PROBE1 | “HUMAN MRNA FOR KIAA0136 GENE, PARTIAL CDS” |
| 235 | 1501858.16_PROBE1 | “RHO-ASSOCIATED COILED-COIL CONTAINING PROTEIN KINASE 1, BINDS RHO AND IS |
| | | INVOLVED IN ACTIN CYTOSKELETON ORGANIZATION, CELL MIGRATION, CYTOKINESIS, |
| | | SUPEROXIDE ANION PRODUCTION, MEMBRANE BLEBBING, AND INTRAHEPATIC METASTASIS |
| | | OF HEPATOCELLULAR CARCINOMA” |
| 236 | AB023198_PROBE1 | “HOMO SAPIENS MRNA FOR KIAA0981 PROTEIN, PARTIAL CDS” |
| 237 | NM_001788.2_PROBE1 | “HOMO SAPIENS CDC10 CELL DIVISION CYCLE 10 HOMOLOG (S. CEREVISIAE) (CDC10), MRNA” |
| 238 | X68560_PROBE1 | H. SAPIENS SPR-2 MRNA FOR GT BOX BINDING PROTEIN |
| 239 | 1251228CB1_PROBE1 | “HOMO SAPIENS SWI/SNF RELATED, MATRIX ASSOCIATED, ACTIN DEPENDENT REGULATOR OF |
| | | CHROMATIN, SUBFAMILY A, MEMBER 5 (SMARCA5), MRNA” |
| 240 | 1888126CB1_PROBE1 | “HOMO SAPIENS RAS HOMOLOG ENRICHED IN BRAIN 2 (RHEB2), MRNA” |
| 241 | 330824.1_PROBE1 | “HOMO SAPIENS CDNA FLJ13675 FIS, CLONE PLACE1011875, HIGHLY SIMILAR TO HOMO SAPIENS |
| | | MRNA FOR KIAA0580 PROTEIN” |
| 242 | NM_001241.1_PROBE1 | “HOMO SAPIENS CYCLIN T2 (CCNT2), TRANSCRIPT VARIANT A, MRNA.” |
| 243 | AB020713_PROBE1 | “HOMO SAPIENS MRNA FOR KIAA0906 PROTEIN, PARTIAL CDS” |
| 244 | AF091095_PROBE1 | “HOMO SAPIENS RPB5-MEDIATING PROTEIN (RMP), TRANSCRIPT VARIANT 1, MRNA” |
| 245 | AF072928_PROBE1 | “HOMO SAPIENS MYOTUBULARIN RELATED PROTEIN 6 MRNA, PARTIAL CDS” |
| 246 | NM_003608.1_PROBE1 | “HOMO SAPIENS G PROTEIN-COUPLED RECEPTOR 65 (GPR65), MRNA.” |
| 247 | NM_003825.1_PROBE1 | “HOMO SAPIENS SYNAPTOSOMAL-ASSOCIATED PROTEIN, 23 KD (SNAP23), TRANSCRIPT |
| | | VARIANT 1, MRNA” |
| 248 | NM_003512.1_PROBE1 | “HOMO SAPIENS H2A HISTONE FAMILY, MEMBER L (H2AFL), MRNA.” |
| 249 | 066892CB1_PROBE1 | “HOMO SAPIENS FRAGILE X MENTAL RETARDATION 1 (FMR1), MRNA” |
| 250 | AB018257_PROBE1 | “HOMO SAPIENS MRNA FOR KIAA0714 PROTEIN, PARTIAL CDS” |
| 251 | NM_018031.2_PROBE1 | “HOMO SAPIENS WD REPEAT DOMAIN 6 (WDR6), TRANSCRIPT VARIANT 1, MRNA.” |
| 252 | 2814863CB1_PROBE1 | “HOMO SAPIENS PRE-B-CELL COLONY-ENHANCING FACTOR (PBEF), MRNA” |
| 253 | NM_025146.1_PROBE1 | “HOMO SAPIENS HYPOTHETICAL PROTEIN FLJ13194 (FLJ13194), MRNA.” |
| 254 | 000106CB1_PROBE1 | “HOMO SAPIENS NADH DEHYDROGENASE (UBIQUINONE) 1 ALPHA SUBCOMPLEX, 4 (9 KD, MLRQ) |
| | | (NDUFA4), MRNA” |
| 255 | 480951.23_PROBE1 | “HOMO SAPIENS YIPPEE PROTEIN (CGI-127), MRNA” |
| 256 | NM_016275.1_PROBE1 | “HOMO SAPIENS SELENOPROTEIN T (LOC51714), MRNA.” |
| 257 | 1985786CB1_PROBE1 | “HOMO SAPIENS PEST-CONTAINING NUCLEAR PROTEIN (PCNP), MRNA” |
| 258 | 1398084.2_PROBE1 | “HOMO SAPIENS HSPC205 MRNA, COMPLETE CDS” |
| 259 | NM_014056.1_PROBE1 | “HOMO SAPIENS DKFZP564K247 PROTEIN (DKFZP564K247), MRNA.” |
| 260 | NM_018184.1_PROBE1 | “HOMO SAPIENS HYPOTHETICAL PROTEIN FLJ10702 (FLJ10702), MRNA.” |
| 261 | NM_016308.1_PROBE1 | “HOMO SAPIENS UMP-CMP KINASE (UMP-CMPK), MRNA.” |
| 262 | NM_016289.1_PROBE1 | “HOMO SAPIENS MO25 PROTEIN (LOC51719), MRNA.” |
| 263 | NM_018695.1_PROBE1 | “HOMO SAPIENS ERBB2 INTERACTING PROTEIN (ERBB2IP), MRNA.” |
| 264 | NM_025238.1_PROBE1 | “HOMO SAPIENS BTB (POZ) DOMAIN CONTAINING 1 (BTBD1), MRNA.” |
| 265 | 4723028CB1_PROBE1 | “HOMO SAPIENS MITOGEN-ACTIVATED PROTEIN KINASE KINASE 1 INTERACTING PROTEIN 1 |
| | | (MAP2K1IP1), MRNA” |
| 266 | NM_013448.1_PROBE1 | “HOMO SAPIENS BROMODOMAIN ADJACENT TO ZINC FINGER DOMAIN, 1A (BAZ1A), MRNA.” |
| 267 | NM_016245.1_PROBE1 | “HOMO SAPIENS RETINAL SHORT-CHAIN DEHYDROGENASE/REDUCTASE RETSDR2 (LOC51170), |
| | | MRNA.” |
| 268 | 1363504.9_PROBE1 | “HOMO SAPIENS HYPOTHETICAL PROTEIN FLJ10154 (FLJ10154), MRNA” |
| 269 | 1452845.8_PROBE1 | HOMO SAPIENS CLONE 25061 MRNA SEQUENCE |
| 270 | NM_018475.1_PROBE1 | “HOMO SAPIENS TPA REGULATED LOCUS (TPARL), MRNA” |
| 271 | NM_016570.1_PROBE1 | “HOMO SAPIENS CDA14 (LOC51290), MRNA.” |
| 272 | AF195514_PROBE1 | “HOMO SAPIENS SUPPRESSOR OF K+ TRANSPORT DEFECT 1 (SKD1), MRNA.” |
| 273 | 444318.1_PROBE1 | “HOMO SAPIENS HYPOTHETICAL PROTEIN FLJ11240 (FLJ11240), MRNA” |
| 274 | 247114.2_PROBE1 | “HOMO SAPIENS HYPOTHETICAL PROTEIN FLJ12910 (FLJ12910), MRNA” |
| 275 | NM_014028.1_PROBE1 | “HOMO SAPIENS HSPC019 PROTEIN (HSPC019), MRNA” |
| 276 | 2508754CB1_PROBE1 | “HOMO SAPIENS ARFAPTIN 1 (HSU52521), MRNA” |
| 277 | NM_016626.1_PROBE1 | “HOMO SAPIENS HYPOTHETICAL PROTEIN (LOC51320), MRNA.” |
| 278 | NM_016618.1_PROBE1 | “HOMO SAPIENS HYPOTHETICAL PROTEIN LOC51315 (LOC51315), MRNA” |
| 279 | NM_022776.1_PROBE1 | “HOMO SAPIENS OXYSTEROL BINDING PROTEIN-LIKE 11 (OSBPL11), MRNA” |
| 280 | NM_017423.1_PROBE1 | “HOMO SAPIENS UDP-N-ACETYL-ALPHA-D-GALACTOSAMINE: POLYPEPTIDE N- |
| | | ACETYLGALACTOSAMINYLTRANSFERASE 7 (GALNAC-T7) (GALNT7), MRNA.” |
| 281 | NM_020651.2_PROBE1 | “HOMO SAPIENS PELLINO HOMOLOG 1 (DROSOPHILA) (PELI1), MRNA.” |
| 282 | NM_017684.1_PROBE1 | “HOMO SAPIENS HYPOTHETICAL PROTEIN FLJ20136 (FLJ20136), MRNA.” |
| 283 | NM_016516.1_PROBE1 | “HOMO SAPIENS TUMOR ANTIGEN SLP-8P (HCC8), MRNA.” |
| 284 | NM_016542.2_PROBE1 | “HOMO SAPIENS SERINE/THREONINE PROTEIN KINASE MASK (MST4), MRNA.” |
| 285 | NM_016603.1_PROBE1 | “HOMO SAPIENS CHROMOSOME 5 OPEN READING FRAME 5 (C5ORF5), MRNA.” |
| 286 | NM_017945.1_PROBE1 | “HOMO SAPIENS HYPOTHETICAL PROTEIN FLJ20730 (FLJ20730), MRNA.” |
| 287 | NM_017768.1_PROBE1 | “HOMO SAPIENS HYPOTHETICAL PROTEIN FLJ20331 (FLJ20331), MRNA” |
| 288 | NM_005767.1_PROBE1 | “HOMO SAPIENS PURINERGIC RECEPTOR (FAMILY A GROUP 5) (P2Y5), MRNA.” |
| 289 | NM_016217.1_PROBE1 | “HOMO SAPIENS HHDC FOR HOMOLOG OF DROSOPHILA HEADCASE (LOC51696), MRNA.” |
| 290 | NM_018169.1_PROBE1 | “HOMO SAPIENS HYPOTHETICAL PROTEIN FLJ10652 (FLJ10652), MRNA” |
| 291 | 234088.1_PROBE1 | “HOMO SAPIENS PHAFIN 2 (FLJ13187), MRNA” |
| 292 | NM_021183.1_PROBE1 | “HOMO SAPIENS HYPOTHETICAL PROTEIN SIMILAR TO SMALL G PROTEINS, ESPECIALLY RAP-2A |
| | | (LOC57826), MRNA.” |
| 293 | NM_021183.1_PROBE1 | “HOMO SAPIENS HYPOTHETICAL PROTEIN SIMILAR TO SMALL G PROTEINS, ESPECIALLY RAP-2A |
| | | (LOC57826), MRNA.” |
| 294 | 289775.3_PROBE1 | “HOMO SAPIENS SIMILAR TO NMDA RECEPTOR-REGULATED GENE 2 (MOUSE) (FLJ11896), MRNA” |
| 295 | NM_014184.1_PROBE1 | “HOMO SAPIENS HSPC163 PROTEIN (HSPC163), MRNA.” |
| 296 | NM_016271.1_PROBE1 | “HOMO SAPIENS STRIN PROTEIN (STRIN), MRNA.” |
| 297 | 001799CB1_PROBE1 | “HOMO SAPIENS HYPOTHETICAL PROTEIN FLJ22557 (FLJ22557), MRNA” |
| 298 | 6158479CB1_PROBE1 | “HOMO SAPIENS COFACTOR REQUIRED FOR SP1 TRANSCRIPTIONAL ACTIVATION, SUBUNIT 3 |
| | | (130 KD) (CRSP3), MRNA” |
| 299 | NM_013352.1_PROBE1 | “HOMO SAPIENS SQUAMOUS CELL CARCINOMA ANTIGEN RECOGNIZED BY T CELL (SART-2), |
| | | MRNA.” |
| 300 | 346874.1_PROBE1 | “HOMO SAPIENS HYPOTHETICAL PROTEIN PRO0082 (PRO0082), MRNA” |
| 301 | 1381955CB1_PROBE1 | “HOMO SAPIENS SIRTUIN SILENT MATING TYPE INFORMATION REGULATION 2 HOMOLOG 1 |
| | | (S. CEREVISIAE) (SIRT1), MRNA” |
| 302 | NM_024558.1_PROBE1 | “HOMO SAPIENS HYPOTHETICAL PROTEIN FLJ13920 (FLJ13920), MRNA.” |
| 303 | NM_022484.1_PROBE1 | “HOMO SAPIENS HYPOTHETICAL PROTEIN FLJ13576 (FLJ13576), MRNA” |
| 304 | NM_020404.1_PROBE1 | “HOMO SAPIENS TUMOR ENDOTHELIAL MARKER 1 PRECURSOR (TEM1), MRNA.” |
| 305 | 2127201CB1_PROBE1 | “HOMO SAPIENS C21ORF19-LIKE PROTEIN (LOC51072), MRNA” |
| 306 | NM_017704.1_PROBE1 | “HOMO SAPIENS HYPOTHETICAL PROTEIN FLJ20189 (FLJ20189), MRNA.” |
| 307 | NM_019083.1_PROBE1 | “HOMO SAPIENS HYPOTHETICAL PROTEIN (FLJ10287), MRNA.” |
| 308 | NM_022768.1_PROBE1 | “HOMO SAPIENS RNA BINDING MOTIF PROTEIN 15 (RBM15), MRNA” |
| 309 | NM_016410.1_PROBE1 | “HOMO SAPIENS HYPOTHETICAL PROTEIN HSPC177 (HSPC177), MRNA.” |
| 310 | NM_017654.1_PROBE1 | “HOMO SAPIENS HYPOTHETICAL PROTEIN FLJ20073 (FLJ20073), MRNA” |
| 311 | NM_015984.1_PROBE1 | “HOMO SAPIENS UBIQUITIN C-TERMINAL HYDROLASE UCH37 (UCH37), MRNA.” |
| 312 | NM_019054.1_PROBE1 | “HOMO SAPIENS HYPOTHETICAL PROTEIN MGC5560 (MGC5560), MRNA.” |
| 313 | NM_025191.1_PROBE1 | “HOMO SAPIENS CHROMOSOME 1 OPEN READING FRAME 22 (C1ORF22), MRNA” |
| 314 | NM_024617.1_PROBE1 | “HOMO SAPIENS HYPOTHETICAL PROTEIN FLJ13409 (FLJ13409), MRNA” |
| 315 | NM_031296.1_PROBE1 | “HOMO SAPIENS RAB33B, MEMBER RAS ONCOGENE FAMILY (RAB33B), MRNA.” |
| 316 | 245378.6_PROBE1 | “HOMO SAPIENS HYPOTHETICAL PROTEIN DC42 (DC42), MRNA” |
| 317 | NM_024116.1_PROBE1 | “HOMO SAPIENS HYPOTHETICAL PROTEIN MGC5306 (MGC5306), MRNA.” |
| 318 | NM_019006.1_PROBE1 | “HOMO SAPIENS PROTEIN ASSOCIATED WITH PRK1 (AWP1), MRNA.” |
| 319 | NM_005171.1_PROBE1 | “HOMO SAPIENS ACTIVATING TRANSCRIPTION FACTOR 1 (ATF1), MRNA.” |
| 320 | NM_018693.1_PROBE1 | “HOMO SAPIENS VITILIGO-ASSOCIATED PROTEIN VIT-1 (VIT1), MRNA” |
| 321 | NM_018307.1_PROBE1 | “HOMO SAPIENS HYPOTHETICAL PROTEIN FLJ11040 (FLJ11040), MRNA.” |
| 322 | 1706512CB1_PROBE1 | “HOMO SAPIENS MRNA FOR KIAA1254 PROTEIN, PARTIAL CDS” |

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein, including patent documents, are incorporated by reference in their entirety.

What is claimed is:

1. A method for diagnosing Huntington's Disease (HD) comprising:
detecting in a biological sample comprising blood from a subject a level of expression of one or more HD biomarker genes,
comparing the level of expression of the one or more HD biomarker genes with a control level of expression of the one or more HD biomarker genes, wherein a difference between the level of expression of the one or more HD biomarker genes in the biological sample and the control level of expression of the one or more HD biomarker genes is diagnostic for HD in the subject.

2. The method of claim 1, wherein the level of expression is detected for 2, 3, 4, 5, 6, 7, 8, 9, 10, 11, 12, 13, 14, 15, 16, 17, 18, 19, 20 or more biomarker genes.

3. The method of claim 1, wherein the subject is asymptomatic for HD.

4. The method of claim 1, wherein the subject is symptomatic for HD.

5. The method of claim 1, wherein the biological sample is a peripheral blood sample.

6. The method of claim 1, wherein the level of expression is detected by determining the amount of MRNA transcribed from the one or more HD biomarker genes.

7. The method of claim 6, wherein the method of determining the amount of mRNA comprises reverse transcription polymerase chain reaction (RT-PCR) amplification.

8. The method of claim 1, wherein the level of expression is detected by determining the amount of a polypeptide encoded by the one or more HD biomarker genes.

9. The method of claim 8, wherein the method of determining the amount of the polypeptide comprises contacting the biological sample with an antibody that specifically binds to a polypeptide encoded by one of the one ore more HD biomarker genes.

10. The method of claim 1, wherein the HD biomarker genes comprise ANXA 1, AXOT, CAPZA1, HIF1A, JJAZ1, P2Y5, PCNP, ROCK1 (p160ROCK), SF3B1, SP3, TAF7 and YIPPEE.

11. The method of claim 1, wherein the HD biomarker genes consist of ANXA1, AXOT, CAPZA1, HIF1A, JJAZ1, P2Y5, PCNP, ROCK1 (p160ROCK), SF3B1, SP3, TAF7 and YIPPEE.

12. The method of claim 1, wherein a higher level of expression of one or more HD biomarker genes in the biological sample compared with the control level of expression of the one or more HD biomarker genes is diagnostic for HD.

13. A method for determining onset, progression, or regression, of Huntington's disease (HD) in a subject, comprising:
detecting in a first biological sample comprising blood from a subject a level of expression of one or more HD biomarker genes,
detecting in a second biological sample comprising blood and obtained from the subject at a time later than the first biological sample, the level of expression of the one or more HD biomarker genes,
comparing the level of expression of the one or more HD biomarker genes in the first biological sample with the level of expression of the one or more HD biomarker genes in the second biological sample, wherein a change in the level between the first biological sample and the second biological sample is an indication of onset, progression, or regression of HD.

14-16. (canceled)

17. The method of claim 13, wherein the biological sample is a peripheral blood sample.

18-21. (canceled)

22. The method of claim 13, wherein the HD biomarker genes comprise ANXA1, AXOT, CAPZA1, HIF1A, JJAZ1, P2Y5, PCNP, ROCK1 (p160ROCK), SF3B1, SP3, TAF7 and YIPPEE.

23-25. (canceled)

26. A method for selecting a course of treatment of a subject having or suspected of having Huntington's disease, comprising:
detecting in a biological sample comprising blood from a subject a level of expression of one or more HD biomarker genes,
comparing the level of expression of the one or more HD biomarker genes to a control level of expression of the one or more HD biomarker genes,
determining the stage and/or type of HD of the subject based on the difference in the level of expression of one or more HD biomarker genes in the sample compared to the level of expression of the one or more HD biomarker genes to the control level of expression, and
selecting a course of treatment for the subject appropriate to the stage and/or type of HD of the subject.

27-29. (canceled)

30. The method of claim 26, wherein the biological sample is a peripheral blood sample.

31-34. (canceled)

35. The method of claim 26, wherein the HD biomarker genes comprise ANXA1, AXOT, CAPZA1, HIF1A, JJAZ1, P2Y5, PCNP, ROCK1 (p160ROCK), SF3B1, SP3, TAF7 and YIPPEE.

36. (canceled)

37. The method of claim 26, wherein a higher level of expression of one or more HD biomarker genes in the biological sample compared with the control level of expression of the one or more HD biomarker genes indicates a more advanced stage and/or more severe type of HD in the biological sample compared with the control.

38. The method of claim 26, wherein the treatment selected is a histone deacetylase (HDAC) inhibitor.

39-161. (canceled)
